(19) United States
(12) Patent Application Publication
(10) Pub. No.: US 2022/0145339 A1
(43) Pub. Date: May 12, 2022

(54) METHOD FOR PREPARATION OF PRIMARY AMINE COMPOUNDS

(71) Applicant: Max-Planck-Gesellschaft zur Förderung der Wissenschaften e.V., Munich (DE)

(72) Inventors: Lennart Schada Von Borzyskowski, Marburg (DE); Tobias Jürgen Erb, Marburg (DE)

(21) Appl. No.: 17/296,559

(22) PCT Filed: Dec. 9, 2019
(86) PCT No.: PCT/EP2019/084291

§ 371 Date: May 25, 2021

(30) Foreign Application Priority Data
Dec. 10, 2018 (EP) 18211454.6

(51) Int. Cl. C12P 13/00

(52) U.S. Cl. CPC C12P 13/001

(57) ABSTRACT

The present invention relates to an enzyme-catalyzed enantioselective method for preparing primary amines from the corresponding imines by using imine reductase enzymes.

### FIELD OF THE INVENTION

The present invention relates to an enzyme-catalyzed enantioselective method for preparing primary amines from the corresponding imines by using imine reductase enzymes.

### BACKGROUND OF THE INVENTION

Chiral amines are valuable building blocks in the pharmaceutical, fine chemical and agricultural industries and are also used in chemical synthesis as chiral auxiliaries or resolving agents for diastereometric salt crystallization. One of the most important reactions for the preparation of chiral amines is the asymmetric reductive amination, wherein the C═N bond is formed in situ by condensation of a carbonyl compound with an amine compound and subsequent asymmetric reduction of the imine intermediate (Lenz et al. World J. Microbiol. Biotechnol. 2017, 33, 199). Chinese patent application no. CN 107935970 A discloses the preparation of 3-methylamino tetrahydrofuran, from tetrahydrofuran-3-carboxaldehyde by reductive amination using a palladium or Raney-Ni catalyst.

In recent years, biocatalysts, particularly imine reductases (IReds) and reductive aminases capable of reducing imines to chiral amines were discovered and structurally characterized. The European patent EP2847214B1 reports on an engineered opine dehydrogenase from Arthrobacter sp. which converts ketones and amines to secondary and tertiary amines under industrially applicable conditions. The reductive amination of cyclohexanone or hydrocinnamic aldehyde with primary alkyl amines by enzymes derived from Amycolatopsis orientalis and Aspergillus oryzae was described by Aleku et al. (ACS Catal. 2016, 6, 3880; Nat. Chem 2017, 9, 961). Huber et al. report on a NADPH-dependent (S)-selective IRed from Streptomyces for the direct reductive amination of ketones (ChemCatChem 2014, 6, 2252) and Hussain et al. describe an IRed for the asymmetric reduction of cyclic imines (ChemCatChem 2015, 7, 579). Wetzl et al. describe imine reductases identified by C-terminal domain clustering of the bacterial protein-sequence space. The enzymes were tested in a model reduction of cyclic imines to pyrrolidines, piperidines, tetrahydroisoquinolines and tetrahydrobenzindoles (ChemBioChem 2015, 16, 1749). However, the known imine reductases were only successful in the synthesis of secondary and tertiary amines. So far, no imine reductase is known in the prior art that catalyzes the reduction of imines to primary amines.

The imine reductases of the prior art possess a high cofactor specificity towards NADPH (nicotinamide adenine dinucleotide phosphate) compared to NADH. However, the concentration of the cofactor NADH in a cell is an order of magnitude higher, NADH is about an order of magnitude less expensive and NADH is more stable than NADPH (J. Biol. Chem. 1971, 246, 1107). Moreover, commonly applied cofactor regeneration systems for NADPH in whole cells are not sufficient on a larger scale (Curr. Opin. Biotechnol. 2003, 14, 421). Therefore, imine reductases having a high specificity for NADH as cofactor are highly desirable for use in in vivo or in vitro reduction of imines on an industrial scale.

It is the objective of the present invention to provide enzyme-catalyzed methods for the preparation of primary amines from the corresponding imines.

The objective of the present invention is solved by the teaching of the independent claims. Further advantageous features, aspects and details of the invention are evident from the dependent claims, the description, the figures, and the examples of the present application.

### DESCRIPTION OF THE INVENTION

The present invention is directed to a method of preparing a primary amine compound of general formula (IB)

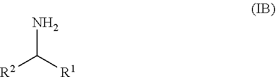

wherein R¹ and R² are independently selected from a hydrogen atom, and optionally substituted alkyl, alkenyl, alkynyl, alkoxy, carboxy, aminocarbonyl, thiocarbonyl, heteroalkyl, heteroalkenyl, heteroalkynyl, carboxyalkyl, aminoalkyl, haloalkyl, alkylthioalkyl, cycloalkyl, aryl, arylalkyl, heterocycloalkyl, heteroaryl, and heteroarylalkyl; and optionally R¹ and R² are linked to form a 3-membered to 10-membered ring;
comprising:
A1) Providing an imine compound of general formula (IIA)

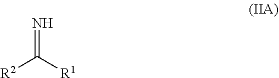

wherein R¹ and R² have the meanings as defined for formula (IB); and
B1) Reacting imine compound of general formula (IIA) with a polypeptide having the enzymatic activity of an imine reductase in the presence of a cofactor, to afford primary amine of general formula (IB),
   wherein the polypeptide comprises the conserved amino acid sequence as set forth in SEQ ID NO: 610.

The method of the present invention relates to the use of an enzyme forming part of the β-hydroxyaspartate pathway (BHAP), which was elucidated by the inventors in proteobacteria, such as in Paracoccus denitrificans.

As shown in FIG. 1, in the BHAP, the enzyme β-hydroxyaspartate aldolase (2) catalyzes the condensation of glycine and glyoxylate to (2R,3S)-β-hydroxyaspartate and the enzyme β-hydroxyaspartate dehydratase (3) catalyzes the subsequent dehydration to iminosuccinate. The iminosuccinate is reduced to aspartate by the iminosuccinate reductase (4) in the presence of the cofactor NADH and the formed aspartate is finally converted with glyoxylate to oxaloacetate in the presence of aspartate-glyoxylate aminotransferase (1).

Based on the sequence of a putative β-hydroxyaspartate aldolase gene (dhaa; GenBank Accession No. AB075600) from Paracoccus denitrificans IFO 1330123, the inventors have identified a homolog in the genome of P. denitrificans DSM413 (BLT64_RS06500), which is part of a gene cluster, consisting of four structural genes and a putative transcriptional regulator. Besides the BHA aldolase (Pden_3919; GenBank: ABL71985, annotated as alanine racemase), the operon comprises ORFs annotated as coding for serine-glyoxylate aminotransferase (Pden_3921, GenBank: ABL71987), serine/threonine dehydratase (Pden_3920, GenBank: ABL71986) and ornithine cyclodeaminase (Pden_3918, GenBank: ABL71984). The putative transcriptional regulator (Pden_3922; annotated as IcIR-family regulator) is in opposite orientation to the four structural genes of the presumed BHAP operon. To verify that these genes indeed encode for the enzymes of the BHAP, the ORFs were cloned and separately overexpressed in Escherichia coli BL21. The purified enzymes were tested in spectrophotometric assays to elucidate their function and confirm their role in the BHAP. The inventors could confirm that Pden_3919 encodes for the key enzyme of the BHAP, BHA aldolase, which catalyzes the condensation of glyoxylate and glycine into ß-hydroxyaspartate. Furthermore, while Pden_3921 is correctly annotated as a PLP-dependent aminotransferase, the inventors could show that its preferred substrates are aspartate and glyoxylate, which are converted into oxaloacetate and glycine, therefore this enzyme has been here named as aspartate-glyoxylate aminotransferase. The function of Pden_3920 as BHA dehydratase, which had previously been purified from cell-free extracts of P. denitrificans (Gibbs and Morris 1965), could be confirmed. Moreover the inventors could show that Pden_3918 encodes for a polypeptide having enzymatic activity of an iminosuccinate reductase (and not ornithine cyclodeaminase as putatively denoted in UniProt database accession no. A1B8Z0), which reduces iminosuccinate to L-aspartate, thereby regenerating the amino group donor in the first step of the BHAP. The inventors could further show by phylogenetic analysis that the BHAP is widespread in α- and γ-proteobacteria, in terrestrial as well as marine habitats.

The conserved amino acid sequence of SEQ ID NO: 610 reflects the conserved sites of the homologous imine reductase enzymes identified by the inventors (SEQ ID Nos.: 300-598) as shown in Table 1 below. The conserved sequence—in its common meaning in the art—is determined from the multiple sequence alignment of the imine reductase sequences, wherein the sites conserved throughout all aligned sequences are listed in Table 1. As the aligned imine reductase sequences may contain gap(s) or deletion(s), the conserved sequence determined from these aligned sequences may also contain gap(s) or deletion(s). Although not explicitly mentioned throughout the disclosure, a skilled person readily envisions that the conserved sequence comprises gap(s) or deletion(s) which were generated by the sequence alignment.

Thus, this conserved amino acid sequence GXKXGX₈GXKXGGX₂PXNX₇NHQSX₃LFX₄GX₈NX₂TAXRTAAX₄SX₃LX₈GX₂GAGXQ X₃QX₁₅WNX₃₉SX₁₅HX₃MGTDTX₂KXEX₁₃DX₃QX₄GEXQX₁₆GX₉RX₆TX₂DGXGX₃QDX A (SEQ ID NO: 610) consists of 251 amino acids or less, wherein each X represents independently of each other exactly one amino acid or a gap. More preferably each X represents independently of each other exactly one proteinogenic amino acids and more preferably exactly one canonic amino acid.

In other words, the present invention is directed to a method of preparing a primary amine compound of general formula (IB)

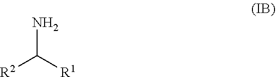

wherein R¹ and R² are independently selected from a hydrogen atom, and optionally substituted alkyl, alkenyl, alkynyl, alkoxy, carboxy, aminocarbonyl, thiocarbonyl, heteroalkyl, heteroalkenyl, heteroalkynyl, carboxyalkyl, aminoalkyl, haloalkyl, alkylthioalkyl, cycloalkyl, aryl, arylalkyl, heterocycloalkyl, heteroaryl, and heteroarylalkyl; and optionally R¹ and R² are linked to form a 3-membered to 10-membered ring;
comprising:
A1) Providing an imine compound of general formula (IIA)

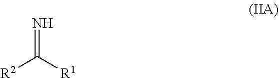

wherein R¹ and R² have the meanings as defined for formula (IB); and
B1) Reacting imine compound of general formula (IIA) with a polypeptide having the enzymatic activity of an imine reductase in the presence of a cofactor, to afford primary amine of general formula (IB),
   wherein the polypeptide comprises the conserved amino acid sequence of GXKXGX₈GXKXGGX₂PXNX₇NHQSX₃LFX₄GX₈NX₂TAXRTAAX₄SX₃LX₈GX₂GAGXQ X₃QX₁₅WNX₃₉SX₁₅HX₃MGTDTX₂KXEX₁₃DX₃QX₄GEXQX₁₆GX₉RX₆TX₂DGXGX₃QDX A (SEQ ID NO: 610), wherein X represents independently for each occurrence an amino acid or a gap.

In one embodiment, the present invention is directed to a method of preparing a primary amine compound of general formula (IB)

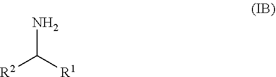

wherein R¹ and R² are independently selected from a hydrogen atom, and optionally substituted alkyl, alkenyl, alkynyl, alkoxy, carboxy, aminocarbonyl, thiocarbonyl, heteroalkyl, heteroalkenyl, heteroalkynyl, carboxyalkyl, aminoalkyl, haloalkyl, alkylthioalkyl, cycloalkyl, aryl, arylalkyl, heterocycloalkyl, heteroaryl, and heteroarylalkyl; and optionally R¹ and R² are linked to form a 3-membered to 10-membered ring;
comprising:
A1) Providing an imine compound of general formula (IIA)

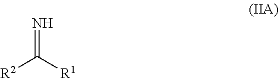

wherein R¹ and R² have the meanings as defined for formula (IB); and
B1) Reacting imine compound of general formula (IIA) with a polypeptide having the enzymatic activity of an imine reductase in the presence of a cofactor, to afford primary amine of general formula (IB),
   wherein the polypeptide comprises the conserved amino acid sequence as set forth in SEQ ID NO: 610, wherein X represents independently for each occurrence an amino acid.

In one embodiment, the present invention is directed to a method of preparing a primary amine compound of general formula (IB)

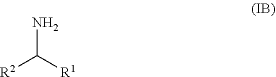

wherein R¹ and R² are independently selected from a hydrogen atom, and optionally substituted alkyl, alkenyl, alkynyl, alkoxy, carboxy, aminocarbonyl, thiocarbonyl, heteroalkyl, heteroalkenyl, heteroalkynyl, carboxyalkyl, aminoalkyl, haloalkyl, alkylthioalkyl, cycloalkyl, aryl, arylalkyl, heterocycloalkyl, heteroaryl, and heteroarylalkyl; and optionally R¹ and R² are linked to form a 3-membered to 10-membered ring;
comprising:
A1) Providing an imine compound of general formula (IIA)

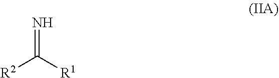

wherein R¹ and R² have the meanings as defined for formula (IB); and
B1) Reacting imine compound of general formula (IIA) with a polypeptide having the enzymatic activity of an imine reductase in the presence of a cofactor, to afford primary amine of general formula (IB),
   wherein the polypeptide comprises the conserved amino acid sequence as set forth in SEQ ID NO: 611.

In one embodiment, the method of preparing a primary amine compound of general formula (IB) comprises:

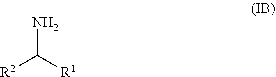

A1) Providing an imine compound of general formula (IIA); and

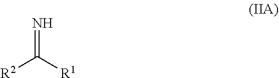

B1) Reacting imine compound of general formula (IIA) with a polypeptide having the enzymatic activity of an imine reductase in the presence of a cofactor, to afford primary amine of general formula (IB),
   wherein the polypeptide comprises a conserved amino acid sequence as set forth in SEQ ID NO: 610; and
   wherein R¹ and R² are independently selected from a hydrogen atom, and optionally substituted linear C₁-C₈ alkyl, branched C₃-C₈ alkyl, linear C₂-C₈ alkenyl, branched C₂-C₈ alkenyl, linear C₂-C₈ alkynyl, branched C₂-C₈ alkynyl, C₃-C₈ cycloalkyl, C₁-C₁₀ heteroaryl, C₂-C₉ heterocyclyl, C₁-C₈ alkoxy, carboxy, aminocarbonyl, thiocarbonyl, C₁-C₈ aminoalkyl, C₁-C₈ aminocarbonylalkyl, C₁-C₈ carboxyalkyl, C₁-C₈ haloalkyl, C₁-C₈ alkylthioalkyl, aryl, C₁-C₈ arylalkyl, C₁-C₈ heterocycloalkyl, heteroaryl, and C₁-C₈ heteroarylalkyl; and optionally R¹ and R² are linked to form a 3-membered to 10-membered ring.

Preferably, the polypeptide comprises a conserved amino acid sequence selected from the sequences as set forth in SEQ ID NO: 611 to 621.

Preferably, R¹ represents —CH₃, —COOR⁵, —COSR⁵, —CSSR⁵, —CONHR⁵, —CONR⁵R⁶, —CH═CH—COOR⁵, —CH═CH—COSR⁵, —CH═CH—CSSR⁵, —CH═CH—CONHR⁵, —CH═CH—CONR⁵R⁶, —C≡C—COOR⁵, —C≡C—COSR⁵, —C≡C—CSSR⁵, —C≡C—CONHR⁵ or —C≡C—CONR⁵R⁶.

Preferably, R² represents —H, —X¹, —CH₃, —CF₃, -Ph, —CH₂Y¹, —CH(Y¹)Y², cyclo-C₃H₅, cyclo-C₄H₇, cyclo-C₅H₉, cyclo-C₆H₁₁, cyclo-C₇H₁₃, Cyclo-C₈H₁₅, —CH(OH)X¹, —C(O)Y¹, —CH(X¹)X², —CH₂CH₂X¹, —(CH₂)₃X¹, —CH₂C(O)X¹ or —C(O)CH₂X¹.

Preferably, X¹ and X² are independently selected from —CN, —COOR⁷, —COOR⁸, —COSR⁷, —COSR⁸, —CSSR⁷, —CSSR⁸, —CONHR⁷, —CONHR⁸, —CONR⁷R⁹ or —CONR⁸R¹⁰.

Preferably, Y¹, Y² and Y³ are independently selected from —X¹, —X², —R¹³, —R¹⁴, or —R¹⁵.

Preferably, R⁵-R¹⁵ represent independently of each other —H, cyclo-C₃H₅, cyclo-C₄H₇, cyclo-C₅H₉, cyclo-C₆H₁₁, cyclo-C₇H₁₃, cyclo-C₈H₁₅, -Ph, —CH₂-Ph, —CPh₃, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)—C(CH₃)₃, —CH═CH₂, —CH₂—CH═CH₂, —C(CH₃)═CH₂, —CH═CH—CH₃, —C₂H₄—CH═CH₂, —C₇H₁₅, —C₈H₁₇, —CH₂—CH═CH—CH₃, —CH═CH—C₂H₅, —CH₂—C(CH₃)═CH₂, —CH(CH₃)—CH═CH, —CH═C(CH₃)₂, —C(CH₃)═CH—CH₃, —CH═CH—CH═CH₂, —C₃H₆—CH═CH₂, —C₂H₄—CH═CH—CH₃, —CH₂—CH═CH—C₂H₅, —CH═CH—C₃H₇, —CH₂—CH═CH—CH═CH₂, —CH═CH—CH═CH—CH₃, —CH₂NH₂, —CH₂OH, —CH₂SH, —CH₂—CH₂NH₂, —CH₂—CH₂SH, —C₆H₄—OCH₃, —C₆H₄—OH, —CH₂—CH₂—OCH₃, —CH₂—CH₂OH, —CH₂—OCH₃, —CH₂—C₆H₄—OCH₃, —CH₂—C₆H₄—OH.

Preferably, R¹ and R² join to form a 5-membered or 6-membered carbocyclic or heterocyclic ring, R¹ and R² being independently selected from:

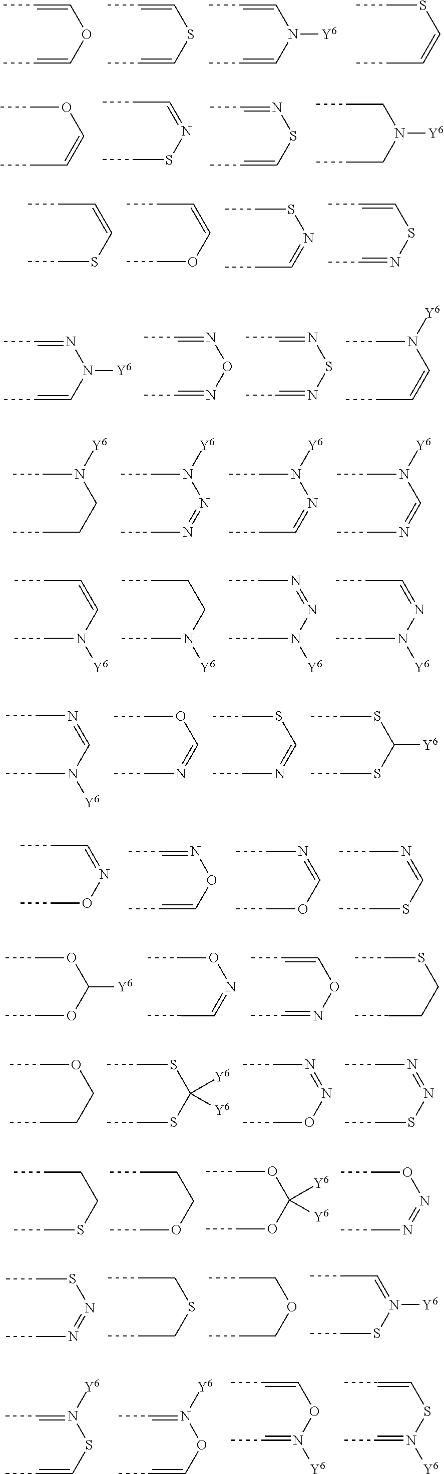

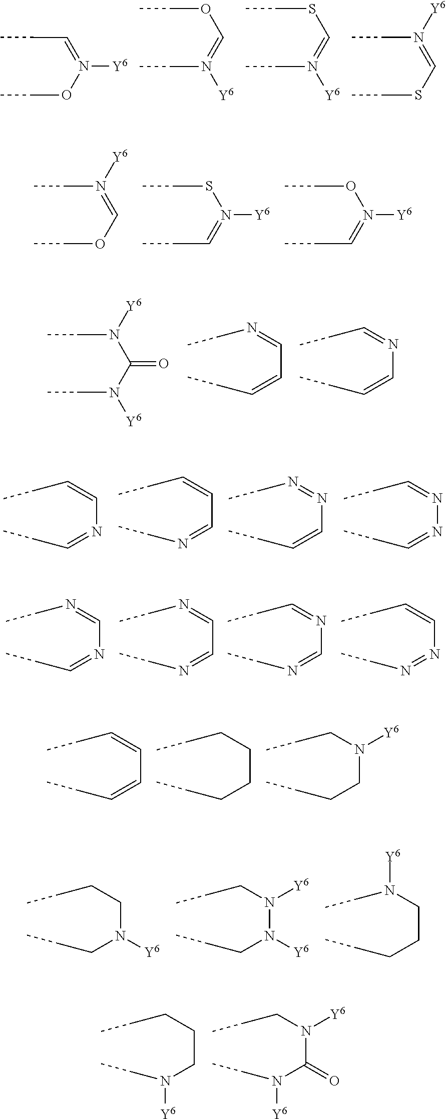

wherein Y⁸ represents —H, —CH₃, —C₂H₅, —C₃H₇, —C₄He, —CH(CH₃)₂, —CH₂CH(CH₃)₂, —CH(CH₃)—C₂H₅, —C(CH₃)₃ or -cyclo-C₃H₅.

In one embodiment of the present invention R¹ and R² are linked to form a 3-membered to 10-membered ring selected from: cyclopropane, cyclobutane, cyclopentane, cyclohexane, cycloheptane, cyclooctane, cyclononane or cylodecane.

Thus, the present invention is also directed to a method of preparing a primary amine compound of general formula (IB), comprising the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises the conserved amino acid sequence as set forth in SEQ ID NO: 610; and
   wherein R¹ represents —CH₃, —COOR⁵, —COSR⁵, —CSSR⁵, —CONHR⁵, —CONR⁵R⁶, —CH═CH—COOR⁵, —CH═CH—COSR⁵, —CH═CH—CSSR⁵, —CH═CH—CONHR⁵, —CH═CH—CONR⁵R⁶, —C≡C—COOR⁵, —C≡C—COSR⁵, —C≡C—CSSR⁵, —C≡C—CONHR⁵ or —C≡C—CONR⁵R⁶;
   R² represents —H, —X¹, —CH₃, —CF₃, -Ph, —CH₂Y¹, —CH(Y¹)Y², cyclo-C₃H₅, cyclo-C₄H₇, cyclo-C₅H, cyclo-C₆H₁₁, cyclo-C₇H₁₃, cyclo-C₈H₁₅, —CH(OH)X¹, —C(O)Y¹, —CH(X¹)X², —CH₂CH₂X¹, —(CH₂)₃X¹, —CH₂C(O)X¹ or —C(O)CH₂X¹;
   X¹ and X² are independently selected from —CN, —COOR⁷, —COOR⁸, —COSR⁷, —COSR⁸, —CSSR⁷, —CSSR⁸, —CONHR⁷, —CONHR⁸, —CONR⁷R⁹ or —CONR⁸R¹⁰;
   Y¹, Y² and Y³ are independently selected from —X¹, —X², —R¹³, —R¹⁴, or —R¹⁵;
   R⁵-R¹⁵ represent independently of each other —H, cyclo-C₃H₅, cyclo-C₄H₇, cyclo-C₅H₉, cyclo-C₆H₁₁, cyclo-C₇H₁₃, cyclo-C₈H₁₅, -Ph, —CH₂-Ph, —CPh₃, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)—C(CH₃)₃, —CH═CH₂, —CH₂—CH═CH₂, —C(CH₃)═CH₂, —CH═CH—CH₃, —C₂H₄—CH═CH₂, —C₇H₁₅, —C₈H₁₇, —CH₂—CH═CH—CH₃, —CH═CH—C₂H₅, —CH₂—C(CH₃)═CH₂, —CH(CH₃)—CH═CH, —CH═C(CH₃)₂, —C(CH₃)═CH—CH₃, —CH═CH—CH═CH₂, —C₃H₆—CH═CH₂, —C₂H₄—CH═CH—CH₃, —CH₂—CH═CH—C₂H₅, —CH═CH—C₃H₇, —CH₂—CH═CH—CH═CH₂, —CH═CH—CH═CH—CH₃, —CH₂NH₂, —CH₂OH, —CH₂SH, —CH₂—CH₂NH₂, —CH₂—CH₂SH, —C₆H₄—OCH₃, —C₆H₄—OH, —CH₂—CH₂—OCH₃, —CH₂—CH₂OH, —CH₂—OCH₃, —CH₂—C₆H₄—OCH₃ or —CH₂—C₆H₄—OH.

Preferably, the method of preparing a primary amine compound of general formula (IB) comprises the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises a conserved amino acid sequence selected from the sequences as set forth in SEQ ID NO 611 to 621; and
   wherein R¹ represents —CH₃, —COOR⁵, —COSR⁵, —CSSR⁵, —CONHR⁵, —CONR⁵R⁶, —CH═CH—COOR⁵, —CH═CH—COSR⁵, —CH═CH—CSSR⁵, —CH═CH—CONHR⁵, —CH═CH—CONR⁵R⁶, —C≡C—COOR⁵, —C≡C—COSR⁵, —C≡C—CSSR⁵, —C≡C—CONHR⁵ or —C≡C—CONR⁵R⁶;
   R² represents —H, —X¹, —CH₃, —CF₃, -Ph, —CH₂Y¹, —CH(Y¹)Y², cyclo-C₃H₅, cyclo-C₄H₇, cyclo-C₅H, cyclo-C₆H₁₁, cyclo-C₇H₁₃, cyclo-C₈H₁₅, —CH(OH)X¹, —C(O)Y¹, —CH(X¹)X², —CH₂CH₂X¹, —(CH₂)₃X¹, —CH₂C(O)X¹ or —C(O)CH₂X¹;
   X¹ and X² are independently selected from —CN, —COOR⁷, —COOR⁸, —COSR⁷, —COSR⁸, —CSSR⁷, —CSSR⁸, —CONHR⁷, —CONHR⁸, —CONR⁷R⁹ or —CONR⁸R¹⁰;
   Y¹, Y² and Y³ are independently selected from —X¹, —X², —R¹³, —R¹⁴, or —R¹⁵;
   R⁵-R¹⁵ represent independently of each other —H, cyclo-C₃H₅, cyclo-C₄H₇, cyclo-C₅H₉, cyclo-C₆H₁₁, cyclo-C₇H₁₃, cyclo-C₈H₁₅, -Ph, —CH₂-Ph, —CPh₃, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)—C(CH₃)₃, —CH═CH₂, —CH₂—CH═CH₂, —C(CH₃)═CH₂, —CH═CH—CH₃, —C₂H₄—CH═CH₂, —C₇H₁₅, —C₈H₁₇, —CH₂—CH═CH—CH₃, —CH═CH—C₂H₅, —CH₂—C(CH₃)═CH₂, —CH(CH₃)—CH═CH, —CH═C(CH₃)₂, —C(CH₃)═CH—CH₃, —CH═CH—CH═CH₂, —C₃H₆—CH═CH₂, —C₂H₄—CH═CH—CH₃, —CH₂—CH═CH—C₂H₅, —CH═CH—C₃H₇, —CH₂—CH═CH—CH═CH₂, —CH═CH—CH═CH—CH₃, —CH₂NH₂, —CH₂OH, —CH₂SH, —CH₂—CH₂NH₂, —CH₂—CH₂SH, —C₆H₄—OCH₃, —C₆H₄—OH, —CH₂—CH₂—OCH₃, —CH₂—CH₂OH, —CH₂—OCH₃, —CH₂—C₆H₄—OCH₃ or —CH₂—C₆H₄—OH.

Preferably, the method of preparing a primary amine compound of general formula (IB) comprises the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises a conserved amino acid sequence as set forth in SEQ ID NO 611; and
   wherein R¹ represents —CH₃, —COOR⁵, —COSR⁵, —CSSR⁵, —CONHR⁵, —CONR⁵R⁶, —CH═CH—COOR⁵, —CH═CH—COSR⁵, —CH═CH—CSSR⁵, —CH═CH—CONHR⁵, —CH═CH—CONR⁵R⁶, —C≡C—COOR⁵, —C≡C—COSR⁵, —C≡C—CSSR⁵, —C≡C—CONHR⁵ or —C≡C—CONR⁵R⁶;
   R² represents —H, —X¹, —CH₃, —CF₃, -Ph, —CH₂Y¹, —CH(Y¹)Y², cyclo-C₃H₅, cyclo-C₄H₇, cyclo-C₅H, cyclo-C₆H₁₁, cyclo-C₇H₁₃, cyclo-C₈H₁₅, —CH(OH)X¹, —C(O)Y¹, —CH(X¹)X², —CH₂CH₂X¹, —(CH₂)₃X¹, —CH₂C(O)X¹ or —C(O)CH₂X¹;
   X¹ and X² are independently selected from —CN, —COOR⁷, —COOR⁸, —COSR⁷, —COSR⁸, —CSSR⁷, —CSSR⁸, —CONHR⁷, —CONHR⁸, —CONR⁷R⁹ or —CONR⁸R¹⁰;
   Y¹, Y² and Y³ are independently selected from —X¹, —X², —R¹³, —R¹⁴, or —R¹⁵;
   R⁵-R¹⁵ represent independently of each other —H, cyclo-C₃H₅, cyclo-C₄H₇, cyclo-C₅H₉, cyclo-C₆H₁₁, cyclo-C₇H₁₃, cyclo-C₈H₁₅, -Ph, —CH₂-Ph, —CPh₃, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₃H₆—CH(CH₃)₂, —C₂H₄—CH(CH₃)—C₂H₅, —CH(CH₃)—C₄He, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)—C(CH₃)₃, —CH═CH₂, —CH₂—CH═CH₂, —C(CH₃)═CH₂, —CH═CH—CH₃, —C₂H₄—CH═CH₂, —C₇H₁₅, —C₈H₁₇, —CH₂—CH═CH—CH₃, —CH═CH—C₂H₅, —CH₂—C(CH₃)═CH₂, —CH(CH₃)—CH═CH, —CH═C(CH₃)₂, —C(CH₃)═CH—CH₃, —CH═CH—CH═CH₂, —C₃H₆—CH═CH₂, —C₂H₄—CH═CH—CH₃, —CH₂—CH═CH—C₂H₅, —CH═CH—C₃H₇, —CH₂—CH═CH—CH═CH₂, —CH═CH—CH═CH—CH₃, —CH₂NH₂, —CH₂OH, —CH₂SH, —CH₂—CH₂NH₂, —CH₂—CH₂SH, —C₆H₄—OCH₃, —C₆H₄—OH, —CH₂—CH₂—OCH₃, —CH₂—CH₂OH, —CH₂—OCH₃, —CH₂—C₆H₄—OCH₃ or —CH₂—C₆H₄—OH.

Preferably, the method of preparing a primary amine compound of general formula (IB) comprises the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises a conserved amino acid sequence as set forth in SEQ ID NO 612; and
   wherein R¹ represents —CH₃, —COOR⁵, —COSR⁵, —CSSR⁵, —CONHR⁵, —CONR⁵R⁶, —CH═CH—COOR⁵, —CH═CH—COSR⁵, —CH═CH—CSSR⁵, —CH═CH—CONHR⁵, —CH═CH—CONR⁵R⁶, —C≡C—COOR⁵, —C≡C—COSR⁵, —C≡C—CSSR⁵, —C≡C—CONHR⁵ or —C≡C—CONR⁵R⁶;
   R² represents —H, —X¹, —CH₃, —CF₃, -Ph, —CH₂Y¹, —CH(Y¹)Y², cyclo-C₃H₅, cyclo-C₄H₇, cyclo-C₅H₉, cyclo-C₆H₁₁, cyclo-C₇H₁₃, cyclo-C₈H₁₅, —CH(OH)X¹, —C(O)Y¹, —CH(X¹)X², —CH₂CH₂X¹, —(CH₂)₃X¹, —CH₂C(O)X¹ or —C(O)CH₂X¹;
   X¹ and X² are independently selected from —CN, —COOR⁷, —COOR⁸, —COSR⁷, —COSR⁸, —CSSR⁷, —CSSR⁸, —CONHR⁷, —CONHR⁸, —CONR⁷R⁹ or —CONR⁸R¹⁰;
   Y¹, Y² and Y³ are independently selected from —X¹, —X², —R¹³, —R¹⁴, or —R¹⁵;
   R⁵-R¹⁵ represent independently of each other —H, cyclo-C₃H₅, cyclo-C₄H₇, cyclo-C₅H₉, cyclo-C₆H₁₁, cyclo-C₇H₁₃, cyclo-C₈H₁₅, -Ph, —CH₂-Ph, —CPh₃, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)—C(CH₃)₃, —CH═CH₂, —CH₂—CH═CH₂, —C(CH₃)═CH₂, —CH═CH—CH₃, —C₂H₄—CH═CH₂, —C₇H₁₅, —C₈H₁₇, —CH₂—CH═CH—CH₃, —CH═CH—C₂H₅, —CH₂—C(CH₃)═CH₂, —CH(CH₃)—CH═CH, —CH═C(CH₃)₂, —C(CH₃)═CH—CH₃, —CH═CH—CH═CH₂, —C₃H₆—CH═CH₂, —C₂H₄—CH═CH—CH₃, —CH₂—CH═CH—C₂H₅, —CH═CH—C₃H₇, —CH₂—CH═CH—CH═CH₂, —CH═CH—CH═CH—CH₃, —CH₂NH₂, —CH₂OH, —CH₂SH, —CH₂—CH₂NH₂, —CH₂—CH₂SH, —C₆H₄—OCH₃, —C₆H₄—OH, —CH₂—CH₂—OCH₃, —CH₂—CH₂OH, —CH₂—OCH₃, —CH₂—C₆H₄—OCH₃ or —CH₂—C₆H₄—OH.

Preferably, the method of preparing a primary amine compound of general formula (IB) comprises the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises a conserved amino acid sequence as set forth in SEQ ID NO 617; and
   wherein R¹ represents —CH₃, —COOR⁵, —COSR⁵, —CSSR⁵, —CONHR⁵, —CONR⁵R⁶, —CH═CH—COOR⁵, —CH═CH—COSR⁵, —CH═CH—CSSR⁵, —CH═CH—CONHR⁵, —CH═CH—CONR⁵R⁶, —C≡C—COOR⁵, —C≡C—COSR⁵, —C≡C—CSSR⁵, —C≡C—CONHR⁵ or —C≡C—CONR⁵R⁶;
   R² represents —H, —X¹, —CH₃, —CF₃, -Ph, —CH₂Y¹, —CH(Y¹)Y², cyclo-C₃H₅, cyclo-C₄H₇, cyclo-C₅H₉, cyclo-C₆H₁₁, cyclo-C₇H₁₃, cyclo-C₈H₁₅, —CH(OH)X¹, —C(O)Y¹, —CH(X¹)X², —CH₂CH₂X¹, —(CH₂)₃X¹, —CH₂C(O)X¹ or —C(O)CH₂X¹; X¹ and X² are independently selected from —CN, —COOR⁷, —COOR⁸, —COSR⁷, —COSR⁸, —CSSR⁷, —CSSR⁸, —CONHR⁷, —CONHR⁸, —CONR⁷R⁹ or —CONR⁸R¹⁰;
   Y¹, Y² and Y³ are independently selected from —X¹, —X², —R¹³, —R¹⁴, or —R¹⁵;
   R⁵-R¹⁵ represent independently of each other —H, cyclo-C₃H₅, cyclo-C₄H₇, cyclo-C₅H₉, cyclo-C₆H₁₁, cyclo-C₇H₁₃, cyclo-C₈H₁₅, -Ph, —CH₂-Ph, —CPh₃, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₃H₆—CH(CH₃)₂, —C₂H₄—CH(CH₃)—C₂H₅, —CH(CH₃)—C₄He, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)—C(CH₃)₃, —CH═CH₂, —CH₂—CH═CH₂, —C(CH₃)═CH₂, —CH═CH—CH₃, —C₂H₄—CH═CH₂, —C₇H₁₅, —C₈H₁₇, —CH₂—CH═CH—CH₃, —CH═CH—C₂H₅, —CH₂—C(CH₃)═CH₂, —CH(CH₃)—CH═CH, —CH═C(CH₃)₂, —C(CH₃)═CH—CH₃, —CH═CH—CH═CH₂, —C₃H₆—CH═CH₂, —C₂H₄—CH═CH—CH₃, —CH₂—CH═CH—C₂H₅, —CH═CH—C₃H₇, —CH₂—CH═CH—CH═CH₂, —CH═CH—CH═CH—CH₃, —CH₂NH₂, —CH₂OH, —CH₂SH, —CH₂—CH₂NH₂, —CH₂—CH₂SH, —C₆H₄—OCH₃, —C₆H₄—OH, —CH₂—CH₂—OCH₃, —CH₂—CH₂OH, —CH₂—OCH₃, —CH₂—C₆H₄—OCH₃ or —CH₂—C₆H₄—OH.

Preferably, the method of preparing a primary amine compound of general formula (IB) comprises the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises a conserved amino acid sequence as set forth in SEQ ID NO 618; and
   wherein R¹ represents —CH₃, —COOR⁵, —COSR⁵, —CSSR⁵, —CONHR⁵, —CONR⁵R⁶, —CH═CH—COOR⁵, —CH═CH—COSR⁵, —CH═CH—CSSR⁵, —CH═CH—CONHR⁵, —CH═CH—CONR⁵R⁶, —C≡C—COOR⁵, —C≡C—COSR⁵, —C≡C—CSSR⁵, —C≡C—CONHR⁵ or —C≡C—CONR⁵R⁶;
   R² represents —H, —X¹, —CH₃, —CF₃, -Ph, —CH₂Y¹, —CH(Y¹)Y², cyclo-C₃H₅, cyclo-C₄H₇, cyclo-C₅H, cyclo-C₆H₁₁, cyclo-C₇H₁₃, cyclo-C₈H₁₅, —CH(OH)X¹, —C(O)Y¹, —CH(X¹)X², —CH₂CH₂X¹, —(CH₂)₃X¹, —CH₂C(O)X¹ or —C(O)CH₂X¹;
   X¹ and X² are independently selected from —CN, —COOR⁷, —COOR⁸, —COSR⁷, —COSR⁸, —CSSR⁷, —CSSR⁸, —CONHR⁷, —CONHR⁸, —CONR⁷R⁹ or —CONR⁸R¹⁰;
   Y¹, Y² and Y³ are independently selected from —X¹, —X², —R¹³, —R¹⁴, or —R¹⁵;
   R⁵-R¹⁵ represent independently of each other —H, cyclo-C₃H₅, cyclo-C₄H₇, cyclo-C₅H₉, cyclo-C₆H₁₁, cyclo-C₇H₁₃, cyclo-C₈H₁₅, -Ph, —CH₂-Ph, —CPh₃, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)—C(CH₃)₃, —CH═CH₂, —CH₂—CH═CH₂, —C(CH₃)═CH₂, —CH═CH—CH₃, —C₂H₄—CH═CH₂, —C₇H₁₅, —C₈H₁₇, —CH₂—CH═CH—CH₃, —CH═CH—C₂H₅, —CH₂—C(CH₃)═CH₂, —CH(CH₃)—CH═CH, —CH═C(CH₃)₂, —C(CH₃)═CH—CH₃, —CH═CH—CH═CH₂, —C₃H₆—CH═CH₂, —C₂H₄—CH═CH—CH₃, —CH₂—CH═CH—C₂H₅, —CH═CH—C₃H₇, —CH₂—CH═CH—CH═CH₂, —CH═CH—CH═CH—CH₃, —CH₂NH₂, —CH₂OH, —CH₂SH, —CH₂—CH₂NH₂, —CH₂—CH₂SH, —C₆H₄—OCH₃, —C₆H₄—OH, —CH₂—CH₂—OCH₃, —CH₂—CH₂OH, —CH₂—OCH₃, —CH₂—C₆H₄—OCH₃ or —CH₂—C₆H₄—OH.

Preferably, the method of preparing a primary amine compound of general formula (IB) comprises the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises a conserved amino acid sequence as set forth in SEQ ID NO 619; and
   wherein R¹ represents —CH₃, —COOR⁵, —COSR⁵, —CSSR⁵, —CONHR⁵, —CONR⁵R⁶, —CH═CH—COOR⁵, —CH═CH—COSR⁵, —CH═CH—CSSR⁵, —CH═CH—CONHR⁵, —CH═CH—CONR⁵R⁶, —C≡C—COOR⁵, —C≡C—COSR⁵, —C≡C—CSSR⁵, —C≡C—CONHR⁵ or —C≡C—CONR⁵R⁶;
   R² represents —H, —X¹, —CH₃, —CF₃, -Ph, —CH₂Y¹, —CH(Y¹)Y², cyclo-C₃H₅, cyclo-C₄H₇, cyclo-C₅H, cyclo-C₆H₁₁, cyclo-C₇H₁₃, cyclo-C₈H₁₅, —CH(OH)X¹, —C(O)Y¹, —CH(X¹)X², —CH₂CH₂X¹, —(CH₂)₃X¹, —CH₂C(O)X¹ or —C(O)CH₂X¹;
   X¹ and X² are independently selected from —CN, —COOR⁷, —COOR⁸, —COSR⁷, —COSR⁸, —CSSR⁷, —CSSR⁸, —CONHR⁷, —CONHR⁸, —CONR⁷R⁹ or —CONR⁸R¹⁰;
   Y¹, Y² and Y³ are independently selected from —X¹, —X², —R¹³, —R¹⁴, or —R¹⁵;
   R⁵-R¹⁵ represent independently of each other —H, cyclo-C₃H₅, cyclo-C₄H₇, cyclo-C₅H₉, cyclo-C₆H₁₁, cyclo-C₇H₁₃, cyclo-C₈H₁₅, -Ph, —CH₂-Ph, —CPh₃, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₃H₆—CH(CH₃)₂, —C₂H₄—CH(CH₃)—C₂H₅, —CH(CH₃)—C₄He, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)—C(CH₃)₃, —CH═CH₂, —CH₂—CH═CH₂, —C(CH₃)═CH₂, —CH═CH—CH₃, —C₂H₄—CH═CH₂, —C₇H₁₅, —C₈H₁₇, —CH₂—CH═CH—CH₃, —CH═CH—C₂H₅, —CH₂—C(CH₃)═CH₂, —CH(CH₃)—CH═CH, —CH═C(CH₃)₂, —C(CH₃)═CH—CH₃, —CH═CH—CH═CH₂, —C₃H₆—CH═CH₂, —C₂H₄—CH═CH—CH₃, —CH₂—CH═CH—C₂H₅, —CH═CH—C₃H₇, —CH₂—CH═CH—CH═CH₂, —CH═CH—CH═CH—CH₃, —CH₂NH₂, —CH₂OH, —CH₂SH, —CH₂—CH₂NH₂, —CH₂—CH₂SH, —C₆H₄—OCH₃, —C₆H₄—OH, —CH₂—CH₂—OCH₃, —CH₂—CH₂OH, —CH₂—OCH₃, —CH₂—C₆H₄—OCH₃ or —CH₂—C₆H₄—OH.

Preferably, the method of preparing a primary amine compound of general formula (IB) comprises the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises a conserved amino acid sequence as set forth in SEQ ID NO 620; and
   wherein R¹ represents —CH₃, —COOR⁵, —COSR⁵, —CSSR⁵, —CONHR⁵, —CONR⁵R⁶, —CH═CH—COOR⁵, —CH═CH—COSR⁵, —CH═CH—CSSR⁵, —CH═CH—CONHR⁵, —CH═CH—CONR⁵R⁶, —C≡C—COOR⁵, —C≡C—COSR⁵, —C≡C—CSSR⁵, —C≡C—CONHR⁵ or —C≡C—CONR⁵R⁶;
   R² represents —H, —X¹, —CH₃, —CF₃, -Ph, —CH₂Y¹, —CH(Y¹)Y², cyclo-C₃H₅, cyclo-C₄H₇, cyclo-C₅H, cyclo-C₆H₁₁, cyclo-C₇H₁₃, cyclo-C₈H₁₅, —CH(OH)X¹, —C(O)Y¹, —CH(X¹)X², —CH₂CH₂X¹, —(CH₂)₃X¹, —CH₂C(O)X¹ or —C(O)CH₂X¹; X¹ and X² are independently selected from —CN, —COOR⁷, —COOR⁸, —COSR⁷, —COSR⁸, —CSSR⁷, —CSSR⁸, —CONHR⁷, —CONHR⁸, —CONR⁷R⁹ or —CONR⁸R¹⁰;
   Y¹, Y² and Y³ are independently selected from —X¹, —X², —R¹³, —R¹⁴, or —R¹⁵;
   R⁵-R¹⁵ represent independently of each other —H, cyclo-C₃H₅, cyclo-C₄H₇, cyclo-C₅H, cyclo-C₆H₁₁, cyclo-C₇H₁₃, cyclo-C₈H₁₅, -Ph, —CH₂-Ph, —CPh₃, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)—C(CH₃)₃, —CH═CH₂, —CH₂—CH═CH₂, —C(CH₃)═CH₂, —CH═CH—CH₃, —C₂H₄—CH═CH₂, —C₇H₁₅, —C₈H₁₇, —CH₂—CH═CH—CH₃, —CH═CH—C₂H₅, —CH₂—C(CH₃)═CH₂, —CH(CH₃)—CH═CH, —CH═C(CH₃)₂, —C(CH₃)═CH—CH₃, —CH═CH—CH═CH₂, —C₃H₆—CH═CH₂, —C₂H₄—CH═CH—CH₃, —CH₂—CH═CH—C₂H₅, —CH═CH—C₃H₇, —CH₂—CH═CH—CH═CH₂, —CH═CH—CH═CH—CH₃, —CH₂NH₂, —CH₂OH, —CH₂SH, —CH₂—CH₂NH₂, —CH₂—CH₂SH, —C₆H₄—OCH₃, —C₆H₄—OH, —CH₂—CH₂—OCH₃, —CH₂—CH₂OH, —CH₂—OCH₃, —CH₂—C₆H₄—OCH₃ or —CH₂—C₆H₄—OH.

Preferably, the method of preparing a primary amine compound of general formula (IB) comprises the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises a conserved amino acid sequence as set forth in SEQ ID NO 621; and
   wherein R¹ represents —CH₃, —COOR⁵, —COSR⁵, —CSSR⁵, —CONHR⁵, —CONR⁵R⁶, —CH═CH—COOR⁵, —CH═CH—COSR⁵, —CH═CH—CSSR⁵, —CH═CH—CONHR⁵, —CH═CH—CONR⁵R⁶, —C≡C—COOR⁵, —C≡C—COSR⁵, —C≡C—CSSR⁵, —C≡C—CONHR⁵ or —C≡C—CONR⁵R⁶;
   R² represents —H, —X¹, —CH₃, —CF₃, -Ph, —CH₂Y¹, —CH(Y¹)Y², cyclo-C₃H₅, cyclo-C₄H₇, cyclo-C₅H, cyclo-C₆H₁₁, cyclo-C₇H₁₃, cyclo-C₈H₁₅, —CH(OH)X¹, —C(O)Y¹, —CH(X¹)X², —CH₂CH₂X¹, —(CH₂)₃X¹, —CH₂C(O)X¹ or —C(O)CH₂X¹;
   X¹ and X² are independently selected from —CN, —COOR⁷, —COOR⁸, —COSR⁷, —COSR⁸, —CSSR⁷, —CSSR⁸, —CONHR⁷, —CONHR⁸, —CONR⁷R⁹ or —CONR⁸R¹⁰;
   Y¹, Y² and Y³ are independently selected from —X¹, —X², —R¹³, —R¹⁴, or —R¹⁵;
   R⁵-R¹⁵ represent independently of each other —H, cyclo-C₃H₅, cyclo-C₄H₇, cyclo-C₅H₉, cyclo-C₆H₁₁, cyclo-C₇H₁₃, cyclo-C₈H₁₅, -Ph, —CH₂-Ph, —CPh₃, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)—C(CH₃)₃, —CH═CH₂, —CH₂—CH═CH₂, —C(CH₃)═CH₂, —CH═CH—CH₃, —C₂H₄—CH═CH₂, —C₇H₁₅, —C₈H₁₇, —CH₂—CH═CH—CH₃, —CH═CH—C₂H₅, —CH₂—C(CH₃)═CH₂, —CH(CH₃)—CH═CH, —CH═C(CH₃)₂, —C(CH₃)═CH—CH₃, —CH═CH—CH═CH₂, —C₃H₆—CH═CH₂, —C₂H₄—CH═CH—CH₃, —CH₂—CH═CH—C₂H₅, —CH═CH—C₃H₇, —CH₂—CH═CH—CH═CH₂, —CH═CH—CH═CH—CH₃, —CH₂NH₂, —CH₂OH, —CH₂SH, —CH₂—CH₂NH₂, —CH₂—CH₂SH, —C₆H₄—OCH₃, —C₆H₄—OH, —CH₂—CH₂—OCH₃, —CH₂—CH₂OH, —CH₂—OCH₃, —CH₂—C₆H₄—OCH₃ or —CH₂—C₆H₄—OH.

More preferably, the invention is directed to a method of preparing a primary amine compound of general formula (IB), wherein amine compound of general formula (IB) is an amino acid or amide, i.e. R¹ represents —COOR⁵, —CONHR⁵ or —CONR⁵R⁶. Thus, in one embodiment of the present invention, the method of preparing an amine compound of general formula (IB) comprises
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises a conserved amino acid sequence as set forth in SEQ ID NO: 610; and
   wherein R¹ represents —COOR⁵, —CONHR⁵ or —CONR⁵R⁶, with R⁵ and R⁶ being independently of each other with represented by —H, cyclo-C₃H₅, cyclo-C₄H₇, cyclo-C₅H₉, cyclo-C₆H₁₁, cyclo-C₇H₁₃, cyclo-C₈H₁₅, -Ph, —CH₂-Ph, —CPh₃, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)—C(CH₃)₃, —CH═CH₂, —CH₂—CH═CH₂, —C(CH₃)═CH₂, —CH═CH—CH₃, —C₂H₄—CH═CH₂, —C₇H₁₅, —C₈H₁₇, —CH₂—CH═CH—CH₃, —CH═CH—C₂H₅, —CH₂—C(CH₃)═CH₂, —CH(CH₃)—CH═CH, —CH═C(CH₃)₂, —C(CH₃)═CH—CH₃, —CH═CH—CH═CH₂, —C₃H₆—CH═CH₂, —C₂H₄—CH═CH—CH₃, —CH₂—CH═CH—C₂H₅, —CH═CH—C₃H₇, —CH₂—CH═CH—CH═CH₂, —CH═CH—CH═CH—CH₃, —CH₂NH₂, —CH₂OH, —CH₂SH, —CH₂—CH₂NH₂, —CH₂—CH₂SH, —C₆H₄—OCH₃, —C₆H₄—OH, —CH₂—CH₂—OCH₃, —CH₂—CH₂OH, —CH₂—OCH₃, —CH₂—C₆H₄—OCH₃ or —CH₂—C₆H₄—OH.

Preferably, the polypeptide comprises a conserved amino acid sequence selected from the sequences as set forth in SEQ ID NO: 611 to 621.

More preferably, the method is directed to the preparation of a primary amine compound of general formula (IB), comprising
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises a conserved amino acid sequence as set forth in SEQ ID NO: 610; and
   wherein the primary amine compound of general formula (IB) is an amino acid, i.e. R¹ represents —COOR⁵ and R² represents —CH(Y¹)Y², wherein Y¹ and Y² have the meanings as defined herein.

In a preferred embodiment of the present invention, R¹ represents —COOH and R² is selected from —CH₃, —COH, —CH₂CH₃, —(CH₂)₂CH₃, —(CH₂)₃CH₃, —CH₂COOH, —CH₂CONH₂, —CH₂CH₂OH, —CH₂CH₂SH, —CH(CH₃)COOH, —(CH₂)₂COOH or —(CH₂)₂CONH₂. Thus, the present invention is directed to a method of preparing a primary amine compound of general formula (IB), comprising the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises a conserved amino acid sequence as set forth in SEQ ID NO: 610; and
   wherein R¹ represents —COOH and R² is selected from —CH₃, —COOH, —CH₂CH₃, —(CH₂)₂CH₃, —(CH₂)₃CH₃, —CH₂COOH, —CH₂CONH₂, —CH₂CH₂OH, —CH₂CH₂SH, —CH(CH₃)COOH, —(CH₂)₂COOH or —(CH₂)₂CONH₂.

Preferably, the method of preparing a primary amine compound of general formula (IB) comprises the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises a conserved amino acid sequence selected from the sequences as set forth in SEQ ID NO: 611 to 621, wherein R¹ represents —COOH and R² is selected from —CH₃, —COOH, —CH₂CH₃, —(CH₂)₂CH₃, —(CH₂)₃CH₃, —CH₂COOH, —CH₂CONH₂, —CH₂CH₂OH, —CH₂CH₂SH, —CH(CH₃)COOH, —(CH₂)₂COOH or —(CH₂)₂CONH₂.

Preferably, the method of preparing a primary amine compound of general formula (IB) comprises the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises a conserved amino acid sequence as set forth in SEQ ID NO: 611, wherein R¹ represents —COOH and R² is selected from —CH₃, —COOH, —CH₂CH₃, —(CH₂)₂CH₃, —(CH₂)₃CH₃, —CH₂COOH, —CH₂CONH₂, —CH₂CH₂OH, —CH₂CH₂SH, —CH(CH₃)COOH, —(CH₂)₂COOH or —(CH₂)₂CONH₂.

Preferably, the method of preparing a primary amine compound of general formula (IB) comprises the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises the conserved amino acid sequence as set forth in SEQ ID NO: 617, wherein R¹ represents —COOH and R² is selected from —CH₃, —COOH, —CH₂CH₃, —(CH₂)₂CH₃, —(CH₂)₃CH₃, —CH₂COOH, —CH₂CONH₂, —CH₂CH₂OH, —CH₂CH₂SH, —CH(CH₃)COOH, —(CH₂)₂COOH or —(CH₂)₂CONH₂.

Preferably, the method of preparing a primary amine compound of general formula (IB) comprises the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises the conserved amino acid sequence as set forth in SEQ ID NO: 618, wherein R¹ represents —COOH and R² is selected from —CH₃, —COOH, —CH₂CH₃, —(CH₂)₂CH₃, —(CH₂)₃CH₃, —CH₂COOH, —CH₂CONH₂, —CH₂CH₂OH, —CH₂CH₂SH, —CH(CH₃)COOH, —(CH₂)₂COOH or —(CH₂)₂CONH₂.

Preferably, the method of preparing a primary amine compound of general formula (IB) comprises the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises the conserved amino acid sequence as set forth in SEQ ID NO: 619, wherein R¹ represents —COOH and R² is selected from —CH₃, —COOH, —CH₂CH₃, —(CH₂)₂CH₃, —(CH₂)₃CH₃, —CH₂COOH, —CH₂CONH₂, —CH₂CH₂OH, —CH₂CH₂SH, —CH(CH₃)COOH, —(CH₂)₂COOH or —(CH₂)₂CONH₂.

Preferably, the method of preparing a primary amine compound of general formula (IB) comprises the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises the conserved amino acid sequence as set forth in SEQ ID NO: 620, wherein R¹ represents —COOH and R² is selected from —CH₃, —COOH, —CH₂CH₃, —(CH₂)₂CH₃, —(CH₂)₃CH₃, —CH₂COOH, —CH₂CONH₂, —CH₂CH₂OH, —CH₂CH₂SH, —CH(CH₃)COOH, —(CH₂)₂COOH or —(CH₂)₂CONH₂.

Preferably, the method of preparing a primary amine compound of general formula (IB) comprises the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises the conserved amino acid sequence as set forth in SEQ ID NO: 621, wherein R¹ represents —COOH and R² is selected from —CH₃, —COOH, —CH₂CH₃, —(CH₂)₂CH₃, —(CH₂)₃CH₃, —CH₂COOH, —CH₂CONH₂, —CH₂CH₂OH, —CH₂CH₂SH, —CH(CH₃)COOH, —(CH₂)₂COOH or —(CH₂)₂CONH₂.

In one embodiment of the present invention, the imine compound of general formula (IIA) is a 2-imino carboxylic acid, preferably a substituted 2-iminopropionate, or substituted 2-iminobutyrate. Preferably, the 2-imino carboxylic acid is selected from the group comprising or consisting of: 2-iminopropionate, 2-iminobutyrate, 2-iminovalerate, 2-iminoglutarate, 2-iminomalonate, 2-imino-4-mercaptobutyrate, 2-imino-3-methylsuccinate, and 2-iminoglutarate or substituted derivatives thereof.

Thus, the present invention is directed to a method of preparing a primary 2-amino carboxylic acid, comprising the steps of:
A1) Providing a 2-imino carboxylic acid; and
B1) Reacting the 2-imino carboxylic acid with a polypeptide having the enzymatic activity of an imine reductase in the presence of a cofactor, to afford a primary 2-amino carboxylic acid,
   wherein the polypeptide comprises a conserved amino acid sequence as set forth in SEQ ID NO: 610; and
   wherein the 2-imino carboxylic acid is selected from the group comprising or consisting of: 2-iminopropionate, 2-iminobutyrate, 2-iminovalerate, 2-iminoglutarate, 2-iminomalonate, 2-imino-4-mercaptobutyrate, 2-imino-3-methylsuccinate, and 2-iminoglutarate or substituted derivatives thereof.

In one embodiment of the present invention the method of preparing a primary amine compound of general formula (IB) comprises the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises a conserved amino acid sequence as set forth in SEQ ID NO: 610, R¹ represents —CH₃, —COOR⁵, —COSR⁵, —CSSR⁵, —CONHR⁵, —CONR⁵R⁶, —CH═CH—COOR⁵, —CH═CH—COSR⁵, —CH═CH—CSSR⁵, —CH═CH—CONHR⁵, —CH═CH—CONR⁵R⁶, —C≡C—COOR⁵, —C≡C—COSR⁵, —C≡C—CSSR⁵, —C≡C—CONHR⁵ or —C≡C—CONR⁵R⁶; with R⁵ and R⁶ having the meanings as defined above;
   R² represents —CHZ¹Z², and wherein Z¹ represents —H or —CH₃ and Z² represents —H, —CH₃, —CH₂CH₃, —(CH₂)₂CH₃, —COOH, —CONH₂, —CH₂OH, —CH₂SH, —CH₂COOH or —CH₂CONH₂

Preferably, the polypeptide comprises a conserved amino acid sequence selected from the sequences as set forth in SEQ ID NO: 611 to 621.

In one embodiment of the present invention the method of preparing a primary amine compound of general formula (IB) comprises the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises a conserved amino acid sequence as set forth in SEQ ID NO: 610, R¹ represents —COOH, R² represents —CHZ¹Z², and wherein Z¹ represents —H or —CH₃ and Z² represents —H, —CH₃, —CH₂CH₃, —(CH₂)₂CH₃, —COOH, —CONH₂, —CH₂OH, —CH₂SH, —CH₂COOH or —CH₂CONH₂.

In one embodiment of the present invention the method of preparing a primary amine compound of general formula (IB) comprises the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises a conserved amino acid sequence as set forth in SEQ ID NO: 610, R¹ represents —COOH and R² represents —CH₂COOH.

Preferably, the polypeptide comprises a conserved amino acid sequence selected from the sequences as set forth in SEQ ID NO: 611 to 621. More preferably, the polypeptide comprises a conserved amino acid sequence as set forth in SEQ ID NO 611. More preferably, the polypeptide comprises a conserved amino acid sequence as set forth in SEQ ID NO 617. More preferably, the polypeptide comprises a conserved amino acid sequence as set forth in SEQ ID NO 618. More preferably, the polypeptide comprises a conserved amino acid sequence as set forth in SEQ ID NO 619. More preferably, the polypeptide comprises a conserved amino acid sequence as set forth in SEQ ID NO 620. More preferably, the polypeptide comprises a conserved amino acid sequence as set forth in SEQ ID NO 621.

Further preferred is a method of preparing a primary amine compound of general formula (IC), i.e. R¹ represents —COOH and R² represents —CHZ¹Z²

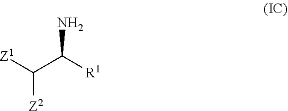

wherein Z¹ represents —H or —CH₃ and Z² represents —H, —CH₃, —CH₂CH₃, —(CH₂)₂CH₃, —COOH, —CONH₂, —CH₂OH, —CH₂SH, —CH₂COOH or —CH₂CONH₂,
comprising the steps of:
A1) Providing an imine compound of general formula (IIC)

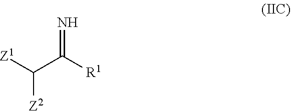

wherein R¹ represents —COOH and Z¹ and Z² have the meanings as defined above; and
B1) Reacting imine compound of general formula (IIC) with a polypeptide having the enzymatic activity of an imine reductase in the presence of a cofactor, to afford primary amine of general formula (IC),
   wherein the polypeptide comprises a conserved amino acid sequence as set forth in SEQ ID NO: 610.

Preferably, the polypeptide comprises a conserved amino acid sequence selected from the sequences as set forth in SEQ ID NO: 611 to 621.

Further preferred is a method of preparing a primary amine compound of general formula (IC), i.e. R¹ represents —CH₃, —COOR⁵, —COSR⁵, —CSSR⁵, —CONHR⁵, —CONR⁵R⁶, —CH═CH—COOR⁵, —CH═CH—COSR⁵, —CH═CH—CSSR⁵, —CH═CH—CONHR⁵, —CH═CH—CONR⁵R⁶, —C≡C—COOR⁵, —C≡C—COSR⁵, —C≡C—CSSR⁵, —C≡C—CONHR⁵ or —C≡C—CONR⁵R⁶, with R⁵ and R⁶ having the meanings as defined above, and R² represents —CHZ¹Z²

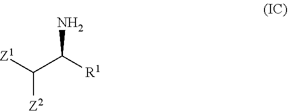

wherein Z¹ represents —H or —CH₃ and Z² represents —H, —CH₃, —CH₂CH₃, —(CH₂)₂CH₃, —COOH, —CONH₂, —CH₂OH, —CH₂SH, —CH₂COOH or —CH₂CONH₂,
comprising the steps of:
A1) Providing an imine compound of general formula (IIC)

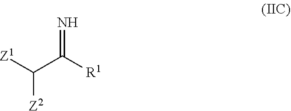

wherein R¹ represents —COOH and Z¹ and Z² have the meanings as defined above; and
B1) Reacting imine compound of general formula (IIC) with a polypeptide having the enzymatic activity of an imine reductase in the presence of a cofactor, to afford primary amine of general formula (IC),
   wherein the polypeptide comprises a conserved amino acid sequence as set forth in SEQ ID NO: 610.

Preferably, the polypeptide comprises a conserved amino acid sequence selected from the sequences as set forth in SEQ ID NO: 611 to 621.

The imine compound of general formula (IIC) of step A1) is preferably provided by reacting amino alcohol compound of general formula (V) with a β-hydroxyaspartate dehydratase

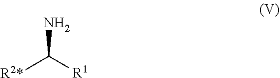

wherein R¹ represents —COOH, R²* represents —C(OH)Z¹Z² and Z¹ and Z² have the meanings as defined herein.

Thus, the present invention is also directed a method of preparing a primary amine compound of general formula (IC), comprising the steps of:
A1) Providing an imine compound of general formula (IIC) by reacting amino alcohol compound of general formula (V) with a β-hydroxyaspartate dehydratase; and
B1) Reacting imine compound of general formula (IIC) with a polypeptide having the enzymatic activity of an imine reductase in the presence of a cofactor, to afford primary amine of general formula (IC),
   wherein the polypeptide comprises a conserved amino acid sequence as set forth in SEQ ID NO: 610; and
   wherein R¹ represents —COOH, Z¹ represents —H or —CH₃ and Z² represents —H, —CH₃, —CH₂CH₃, —(CH₂)₂CH₃, —COOH, —CONH₂, —CH₂OH, —CH₂SH, —CH₂COOH or —CH₂CONH₂.

In other words, the present invention is directed to a method of preparing a primary amine compound of general formula (IC), wherein R¹ represents —COOH, Z¹ represents —H or —CH₃ and Z² represents —H, —CH₃, —CH₂CH₃, —(CH₂)₂CH₃, —COOH, —CONH₂, —CH₂OH, —CH₂SH, —CH₂COOH or —CH₂CONH₂, comprising:

A1″a) Providing an amino alcohol compound of general formula (V),
A1″b) Reacting amino alcohol compound of general formula (V) with a polypeptide having the enzymatic activity of a β-hydroxyaspartate dehydratase, to afford a primary imine compound of general formula (IIC),
and
B1″) Reacting imine compound of general formula (IIC) with a polypeptide having the enzymatic activity of an imine reductase in the presence of a cofactor, to afford a primary amine of general formula (IC),
wherein the polypeptide comprises a conserved amino acid sequence as set forth in SEQ ID NO: 610.

Preferably, the polypeptide comprises a conserved amino acid sequence selected from the sequences as set forth in SEQ ID NO: 611 to 621. More preferably, the polypeptide comprises a conserved amino acid sequence as set forth in SEQ ID NO 611. More preferably, the polypeptide comprises a conserved amino acid sequence as set forth in SEQ ID NO 617. More preferably, the polypeptide comprises a conserved amino acid sequence as set forth in SEQ ID NO 618. More preferably, the polypeptide comprises a conserved amino acid sequence as set forth in SEQ ID NO 619. More preferably, the polypeptide comprises a conserved amino acid sequence as set forth in SEQ ID NO 620. More preferably, the polypeptide comprises a conserved amino acid sequence as set forth in SEQ ID NO 621.

Preferably, the method of preparing a primary amine compound of general formula (IC) comprises:

A1″a) Providing an amino alcohol compound of general formula (V),

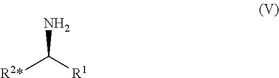

wherein R¹ represents —COOH, R²* represents —C(OH)Z¹Z², Z¹ represents —H or —CH₃ and Z² represents —H, —CH₃, —CH₂CH₃, —(CH₂)₂CH₃, —COOH, —CONH₂, —CH₂OH, —CH₂SH, —CH₂COOH or —CH₂CONH₂;
A1″b) Reacting amino alcohol compound of general formula (V) with a polypeptide having the enzymatic activity of a β-hydroxyaspartate dehydratase, to afford a primary imine compound of general formula (IIC); and
B1″) Reacting imine compound of general formula (IIC) with a polypeptide comprising an amino acid sequence of at least 80% sequence identity to an amino acid sequence selected from SEQ ID Nos: 300-598 and which has the enzymatic activity of an imine reductase in the presence of the cofactor NADH, to afford an amine of general formula (IC).

Preferably, the method of preparing a primary amine compound of general formula (IC) comprises:

A1″a) Providing an amino alcohol compound of general formula (V),
wherein R¹ represents —COOH, R²* represents —C(OH)Z¹Z², Z¹ represents —H or —CH₃ and Z² represents —H, —CH₃, —CH₂CH₃, —(CH₂)₂CH₃, —COOH, —CONH₂, —CH₂OH, —CH₂SH, —CH₂COOH or —CH₂CONH₂;
A1″b) Reacting amino alcohol compound of general formula (V) with a polypeptide comprising an amino acid sequence of at least 80% sequence identity to SEQ ID NO: 602 having the enzymatic activity of a β-hydroxyaspartate dehydratase, to afford a primary imine compound of general formula (IIC); and
B1″) Reacting imine compound of general formula (IIC) with a polypeptide comprising an amino acid sequence of at least 80% sequence identity to an amino acid sequence selected from SEQ ID Nos: 300-598 and which has the enzymatic activity of an imine reductase in the presence of the cofactor NADH, to afford an amine of general formula (IC).

Preferably, in the inventive methods described herein, the amino alcohol compound has the general formula (V), wherein R¹ represents —COOH, Z¹ represents —H and Z² represents —CH₂COOH.

Preferably, in step B1″) of the inventive method, the polypeptide comprises an amino acid sequence selected from SEQ ID NO: 300, 306, 321, 324, 325, 338, 346, 357, 374, 422, 434 and 459 or at least 80% sequence identity of the aforementioned.

More preferably, in step B1″) of the inventive method, the polypeptide comprises an amino acid sequence as set forth in SEQ ID NO 434 or at least 80% sequence identity of the aforementioned.

### DETAILED DESCRIPTION OF THE INVENTION

### Definitions

“Iminosuccinate reductase activity” as used herein, refers to an enzymatic activity in which the imine group of iminosuccinate is reduced to a primary amine group and asparte is formed.

“Iminosuccinate reductase” as used herein refers to a polypeptide having iminosuccinate reductase activity. Iminosuccinate reductase includes but is not limited to enzymes comprising the conserved amino acid sequence of GXKXGX₈GXKXGGX₂PXNX₇NHQSX₃LFX₄GX₈NX₂TAXRTAAX₄SX₃LX₈GX₂GAGXQ X₃QX₁₅WNX₃₉SX₁₅HX₃MGTDTX₂KXEX₁₃DX₃QX₄GEXQX₁₆GX₉RX₆TX₂DGXGX₃QDX A (SEQ ID NO: 610) as well as enzymes from proteobacteria (SEQ ID NO: 300-598), such as Paracoccus denitrificans (SEQ ID NO: 434).

“Imine reductase activity” as used herein refers to an enzymatic activity in which an imine group of an imine compound is reduced to a primary amine product compound, in the presence of cofactor NADH or NADPH as illustrated in FIG. 2C.

The newly identified group of enzymes called imine reductases or even more precisely primary imine reductases convert only primary imines to primary amines (as shown in FIG. 2C) but not secondary imines to secondary amines (as shown in FIG. 2B) and are also not able to convert ketones to tertiary amines (as shown in FIG. 2A). As used herein the term imine or primary imine refers to imines having a hydrogen attached to the nitrogen atom and are represented by the general formula

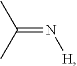

while secondary imines do not have a hydrogen atom bound to the nitrogen atom and are represented by the following general formula

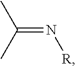

wherein R is different from hydrogen.

In the state of the art imine reductases or even more precisely secondary imine reductases are known which catalyze the reduction of secondary imines to the corresponding secondary amines as shown in FIG. 2B, but which do not have primary imine reductase activity as shown in FIG. 2C.

The newly identified primary imine reductases catalyze the reduction of primary imines to the corresponding primary amines as shown in FIG. 2C, but do not have secondary imine reductase activity as shown in FIG. 2B and do also not catalyze the reductive amination of a carbonyl compound and an amine substrate as illustrated in FIG. 2A.

Consequently, the term “Imine reductase activity” as used herein refers only to the enzymatic activity of catalyzing the reduction of primary imines to the corresponding primary amines as shown in FIG. 2C, excluding the activity to catalyze the reduction of secondary imines to the corresponding secondary amines as shown in FIG. 2B and excluding the activity to catalyze the reductive amination of a carbonyl compound and an amine substrate as illustrated in FIG. 2A.

“Imine reductase” as used herein refers to a polypeptide having an imine reductase activity. It is to be understood that imine reductases are not limited to polypeptide variants derived from the naturally occurring imine reductases from various bacteria, such as Paracoccus denitrificans, but may include other enzymes having imine reductase activity, or recombinant variants of the naturally occurring imine reductases, including but not limiting enzymes comprising the conserved amino acid sequence of GXKXGX₈GXKXGGX₂PXNX₇NHQSX₃LFX₄GX₈NX₂TAXRTAAX₄SX₃LX₈GX₂GAGXQ X₃QX₁₅WNX₃₉SX₁₅HX₃MGTDTX₂KXEX₁₃DX₃QX₄GEXQX₁₆GX₉RX₆TX₂DGXGX₃QDX A (SEQ ID NO: 610), as well as enzymes from proteobacteria (SEQ ID NO: 300-598), such as Paracoccus denitrificans (SEQ ID NO: 434). Accordingly, a polypeptide having iminosuccinate reductase activity is also a polypeptide having imine reductase activity.

Thus, within this conserved amino acid sequence of 251 amino acids, each X represents independently of each other exactly one amino acid and preferably one proteinogenic amino acids and more preferably exactly one canonic amino acid.

As used herein, “β-hydroxyaspartate dehydratase” refers to a polypeptide having a β-hydroxyaspartate dehydratase activity, i.e. a polypeptide that catalyzes the reaction of β-hydroxyaspartate and derivatives thereof to iminosuccinate and the corresponding derivatives thereof. The β-hydroxyaspartate dehydratase belongs to the EC class 4.3.1.20.

“Percentage of sequence identity” and “percentage homology” are used interchangeably herein to refer to comparisons among nucleic acids and polypeptides, and are determined by comparing two optimally aligned sequences over a comparison window, wherein the portion of the nucleic acids or polypeptide sequence in the comparison window may comprise additions or deletions (i.e. gaps) as compared to the reference sequence for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences.

“Reference sequence” refers to a defined sequence used as a basis for a sequence comparison. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, or the full length of the nucleic acid or polypeptide. Since two nucleic acids or polypeptides may each (1) comprise a sequence (i.e., a portion of the complete sequence) that is similar between the two sequences, and (2) may further comprise a sequence that is divergent between the two sequences, sequence comparisons between two (or more) nucleic acids or polypeptide are typically performed by comparing sequences of the two nucleic acids or polypeptides over a “comparison window” to identify and compare local regions of sequence similarity. In some embodiments, a “reference sequence” can be based on a primary amino acid sequence, where the reference sequence is a sequence that can have one or more changes in the primary sequence.

“Substantial identity” refers to a nucleic acid or polypeptide sequence that has at least 80 percent sequence identity, at least 85 percent identity and 89 to 95 percent sequence identity, more usually at least 99 percent sequence identity as compared to a reference sequence over a comparison window of at least 20 residue positions, frequently over a window of at least 30-50 residues, wherein the percentage of sequence identity is calculated by comparing the reference sequence to a sequence that includes deletions or additions which total 20 percent or less of the reference sequence over the window of comparison. In specific embodiments applied to polypeptides, the term “substantial identity” means that two polypeptide sequences, when optimally aligned, share at least 80 percent sequence identity, preferably at least 89 percent sequence identity, at least 95 percent sequence identity or more (e.g., 99 percent sequence identity). Preferably, residue positions which are not identical differ by conservative amino acid substitutions.

“Deletion” refers to modification to the polypeptide by removal of one or more amino acids from the reference polypeptide. Deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the reference enzyme while retaining enzymatic activity and/or retaining the improved properties of an engineered imine reductase enzyme. Deletions can be directed to the internal portions and/or terminal portions of the polypeptide. In various embodiments, the deletion can comprise a continuous segment or can be discontinuous.

“Insertion” refers to modification to the polypeptide by addition of one or more amino acids from the reference polypeptide. In some embodiments, the improved engineered imine reductase enzymes comprise insertions of one or more amino acids to the naturally occurring polypeptide having imine reductase activity as well as insertions of one or more amino acids to other improved imine reductase polypeptides. Insertions can be in the internal portions of the polypeptide, or to the carboxy or amino terminus. Insertions as used herein include fusion proteins as is known in the art. The insertion can be a contiguous segment of amino acids or separated by one or more of the amino acids in the naturally occurring polypeptide.

“Isolated polypeptide” refers to a polypeptide which is substantially separated from other contaminants that naturally accompany it, e.g., protein, lipids, and nucleic acids. The term embraces polypeptides which have been removed or purified from their naturally-occurring environment or expression system (e.g., host cell or in vitro synthesis). The imine reductase enzymes may be present within a cell, present in the cellular medium, or prepared in various forms, such as lysates or isolated preparations. As such, in some embodiments, the polypeptide having the enzymatic activity of an imine reductase enzyme can be an isolated polypeptide.

“Improved enzyme property” refers to a polypeptide having the enzymatic activity of an imine reductase that exhibits an improvement in any enzyme property as compared to a reference imine reductase. For the polypeptides having the enzymatic activity of an imine reductase described herein, the comparison is generally made to the wild-type enzyme from which the imine reductase is derived, although in some embodiments, the reference enzyme can be another improved engineered imine reductase. Enzyme properties for which improvement is desirable include, but are not limited to, enzymatic activity (which can be expressed in terms of percent conversion of the substrate), thermo stability, solvent stability, pH activity profile, cofactor requirements, refractoriness to inhibitors (e.g., substrate or product inhibition), stereospecificity, and stereoselectivity (including enantioselectivity).

“Increased enzymatic activity” refers to an improved property of the polypeptides having the enzymatic activity of an imine reductase, which can be represented by an increase in specific activity (e.g., product produced/time/weight protein) or an increase in percent conversion of the substrate to the product (e.g., percent conversion of starting amount of substrate to product in a specified time period using a specified amount of imine reductase) as compared to the reference imine reductase enzyme. Any property relating to enzyme activity may be affected, including the classical enzyme properties of Kₘ, Vₘₐₓ or k_{cat}, changes of which can lead to increased enzymatic activity. Improvements in enzyme activity can be from about 1.2 times the enzymatic activity of the corresponding wild-type enzyme, to as much as 2 times, 5 times, 10 times, 20 times, 25 times, 50 times or more enzymatic activity than the naturally occurring or another engineered imine reductase from which the imine reductase polypeptides were derived. Imine reductase activity can be measured by any one of standard assays, such as by monitoring changes in properties of substrates, cofactors, or products. In some embodiments, the amount of products generated can be measured by Liquid Chromatography-Mass Spectrometry (LC-MS). Comparisons of enzyme activities are made using a defined preparation of enzyme, a defined assay under a set condition, and one or more defined substrates, as further described in detail herein. Generally, when lysates are compared, the numbers of cells and the amount of protein assayed are determined as well as use of identical expression systems and identical host cells to minimize variations in amount of enzyme produced by the host cells and present in the lysates.

“Suitable reaction conditions” refer to those conditions in the biocatalytic reaction solution (e.g., ranges of enzyme loading, substrate loading, cofactor loading, temperature, pH, buffers, co-solvents, etc.) under which a polypeptide having imine reductase activity of the present invention is capable of converting a substrate compound to a product compound (e.g. conversion of compound of formula (IIA) to a compound of formula (IB). Exemplary “suitable reaction conditions” are provided in the present disclosure and illustrated by the Examples.

“Cofactor regeneration system” or “cofactor recycling system” refers to a set of reactants that participate in a reaction that reduces the oxidized form of the cofactor (e.g., NADP⁺ to NADPH). Cofactors oxidized by the imine reductase catalyzed reductive amination of a ketone substrate are regenerated in reduced form by the cofactor regeneration system. Cofactor regeneration systems comprise a stoichiometric reductant that is a source of reducing hydrogen equivalents and is capable of reducing the oxidized form of the cofactor. The cofactor regeneration system may further comprise a catalyst, for example an enzyme catalyst that catalyzes the reduction of the oxidized form of the cofactor by the reductant. Cofactor regeneration systems to regenerate NADH from NAD⁺ or NADPH from NADP⁺, respectively, are known in the art and may be used in the methods described herein.

“Formate dehydrogenase” and “FDH” are used interchangeably herein to refer to an NAD⁺ or NADP⁺-dependent enzyme that catalyzes the conversion of formate and NAD⁺ or NADP⁺ to carbon dioxide and NADH or NADPH, respectively.

“Alkyl” refers to saturated hydrocarbon groups of from 1 to 18 carbon atoms, either straight chained or branched, more preferably from 1 to 8 carbon atoms, and most preferably 1 to 6 carbon atoms. An alkyl with a specified number of carbon atoms is denoted as C₁-C₈ alkyl and refers to a linear C₁-C₈ alkyl of —CH₃, —C₂H₅, —C₃H₇, —C₄H₉, —C₅H₁₁, —C₆H₁₃, —C₇H₁₅, —C₈H₁₇, —CH₂-Ph, —CH₂—CH₂-Ph or a branched C₁-C₈ alkyl or preferably branched C₃-C₈ alkyl of —CH(CH₃)₂, —CH₂—CH(CH₃)₂, —CH(CH₃)—C₂H₅, —C(CH₃)₃, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)—C(CH₃)₃, —C₄H₈—CH(CH₃)₂, —C₃H₆—CH(CH₃)—C₂H₅, —C₃H₆—CH(CH₃)—C₂H₅, —C₂H₄—CH(CH₃)—C₃H₇, —CH₂—CH(CH₃)—C₄H₉, —CH(CH₃)—C₅H₁₁, —CH(C₂H₅)—C₄H₉, —C₂H₄—CH(CH₃)—C₃H₇, —CH₂—CH(C₂H₅)—C₃H₇, —CH₂—CH(CH₃)—C₄H₉, —CH₂—CH(CH₃)—CH₂—CH(CH₃)₂, —CH(C₂H₅)—CH₂—CH(CH₃)₂, —CH(CH₃)—C₂H₄—CH(CH₃)₂, —CH(CH₃)—CH₂—CH(CH₃)—C₂H₅, —CH₂—CH(CH₃)—CH(CH₃)—C₂H₅, —CH(CH₃)—CH₂—CH(CH₃)—C₂H₅, —CH(CH₃)—CH(C₂H₅)—C₂H₅, —CH(C₂H₅)—CH(CH₃)—C₂H₅, —CH(CH₃)—CH(CH₃)—C₃H₇, —C₂H₄—CH(CH₃)—CH(CH₃)₂, —CH₂—CH(C₂H₅)—CH(CH₃)₂, —CH₂—CH(CH₃)—CH₂—CH(CH₃)₂, —CH₂—CH(CH₃)—CH(CH₃)—C₂H₅, —C₂H₄—C(CH₃)₂—C₂H₅, —CH₂—C(CH₃)(C₂H₅)₂, —CH₂—C(CH₃)₂—C₃H₇, —CH₂—C(CH₃)₂—C₃H₇, —C(CH₃)(C₂H₅)—C₃H₇, —C(CH₃)₂—C₄H₉, —CH₂—C(CH₃)₂—CH(CH₃)₂, —C(CH₃)(C₂H₅)—CH(CH₃)₂, —C(CH₃)₂—CH₂—CH(CH₃)₂, —C(CH₃)₂—C(CH₃)₃, —C(CH₃)₂—CH(CH₃)—C₂H₅, —C₃H₆—C(CH₃)₃, —C₂H₄—C(CH₃)₂—C₂H₅, —CH₂—CH(CH₃)—C(CH₃)₃, —CH(C₂H₅)—C(CH₃)₃, —CH(CH₃)—CH₂—C(CH₃)₃, —CH(CH₃)—C(CH₃)₂—C₂H₅, —C₅H₁₀—CH(CH₃)₂, —C₄H₈—C(CH₃)₃, —C₄H₈—CH(CH₃)—C₂H₅, —C₄H₈—CH(CH₃)—C₂H₅, —C₃H₆—C(CH₃)₂—C₂H₅, —C₃H₆—CH(C₂H₅)—C₂H₅, —C₃H₆—CH(CH₃)—C₃H₇, —C₂H₄—C(CH₃)₂—C₃H₇, —C₂H₄—CH(C₂H₅)—C₃H₇, —C₂H₄—CH(CH₃)—C₄H₉, —CH₂—C(CH₃)₂—C₄H₉, —CH₂—CH(C₂H₅)—C₄H₉, —CH₂—CH(CH₃)—C₅H₁₁, —C(CH₃)₂—C₅H₁₁, —CH(CH₃)—C₆H₁₃, —CH(C₃H₇)—C₄H₉, —CH(C₂H₅)—C₅H₁₁, —CH₂—C(CH₃)(C₂H₅)—C₃H₇, —C₂H₄—CH(CH₃)—CH₂—CH(CH₃)₂, —CH₂—C(CH₃)₂—CH₂—CH(CH₃)₂, —CH₂—CH(C₂H₅)—CH₂—CH(CH₃)₂, —CH₂—CH(CH₃)—C₂H₄—CH(CH₃)₂, —CH₂—CH(CH₃)—CH₂—C(CH₃)₃, —CH₂—CH(CH₃)—CH₂—CH(CH₃)—C₂H₅, —C(CH₃)(C₂H₅)—CH₂—CH(CH₃)₂, —CH(C₃H₇)—CH₂—CH(CH₃)₂, —CH(C₂H₅)—C₂H₄—CH(CH₃)₂, —CH(C₂H₅)—CH₂—C(CH₃)₃, —CH(C₂H₅)—CH₂—CH(CH₃)—C₂H₅, —CH₂—CH(CH₃)—C₂H₄—CH(CH₃)₂, —C(CH₃)₂—C₂H₄—CH(CH₃)₂, —CH(C₂H₅)—C₂H₄—CH(CH₃)₂, —CH(CH₃)—C₃H₆—CH(CH₃)₂, —CH(CH₃)—C₂H₄—C(CH₃)₃, —CH(CH₃)—C₂H₄—CH(CH₃)—C₂H₅, —CH₂—CH(CH₃)—CH₂—CH(CH₃)—C₂H₅, —C(CH₃)₂—CH₂—CH(CH₃)—C₂H₅, —CH(CH₃)—C₂H₄—CH(CH₃)—C₂H₅, —CH(CH₃)—CH₂—C(CH₃)₂—C₂H₅, —CH(CH₃)—CH₂—CH(CH₃)—C₃H₇, —C₂H₄—CH(CH₃)—CH(CH₃)—C₂H₅, —CH₂—C(CH₃)₂—CH(CH₃)—C₂H₅, —CH₂—CH(C₂H₅)—CH(CH₃)—C₂H₅, —CH₂—CH(CH₃)—CH₂—CH(CH₃)—C₂H₅, —CH₂—CH(CH₃)—C(CH₃)₂—C₂H₅, —CH₂—CH(CH₃)—CH(C₂H₅)₂, —C₃H₆—CH(CH₃)—CH(CH₃)₂, —C₂H₄—C(CH₃)₂—CH(CH₃)₂, —C₂H₄—CH(C₂H₅)—CH(CH₃)₂, —C₂H₄—CH(CH₃)—C(CH₃)₃, —C₂H₄—CH(CH₃)—CH(CH₃)—C₂H₅, —C₃H₆—C(CH₃)₂—C₂H₅, —C₂H₄—C(CH₃)₂—C₃H₇, —CH₂—C(CH₃)(C₂H₅)₂, —C₂H₄—C(C₂H₅)₃, —C₂H₄—C(CH₃)₂—C₃H₇, —CH₂—C(CH₃)₂—C₄H₉, —C(C₂H₅)₂—C₃H₇, —C(CH₃)(C₃H₇)—C₃H₇, —C(CH₃)(C₂H₅)—C₄H₉, —C(CH₃)(—C₂H₅)—C₄H₉, —C(CH₃)₂—C₅H₁₁, —C₂H₄—C(CH₃)₂—CH(CH₃)₂, —CH₂—C(CH₃)₂—C(CH₃)₃, —C(C₂H₅)₂—CH(CH₃)₂, —C(CH₃)(C₃H₇)—CH(CH₃)₂, —C(CH₃)(C₂H₅)—C(CH₃)₃, —CH₂—C(CH₃)₂—CH₂—CH(CH₃)₂, —C(CH₃)₂—C₂H₄—CH(CH₃)₂, —C(CH₃)₂—CH₂—C(CH₃)₃, —CH₂—C(CH₃)₂—C(CH₃)₃, —C₄H₈—C(CH₃)₃, —C₃H₆—C(CH₃)₂—C₂H₅, —C₂H₄—C(CH₃)₂—C₃H₇, —C₂H₄—CH(CH₃)—C(CH₃)₃, —CH₂—C(CH₃)₂—C(CH₃)₃.

“Alkylene” refers to a straight or branched chain divalent hydrocarbon radical having from 1 to 18 carbon atoms, more preferably from 1 to 8 carbon atoms, and most preferably 1 to 6 carbon atoms.

“Alkenyl” refers to groups of from 2 to 12 carbon atoms, preferably 2 to 8 carbon atoms (C₂-C₈ alkenyl), either straight or branched containing at least one double bond but optionally containing more than one double bond. As used herein, the term “linear or branched C₂-C₈ alkenyl” refers to —CH═CH₂, —CH₂—CH═CH₂, —C(CH₃)═CH₂, —CH═CH—CH₃, —C₂H₄—CH═CH₂, —CH═CH—C₂H₅, —CH₂—C(CH₃)═CH₂, —CH(CH₃)—CH═CH, —CH═C(CH₃)₂, —C(CH₃)═CH—CH₃, —CH═CH—CH═CH₂, —C₃H₆—CH═CH₂, —C₂H₄—CH═CH—CH₃, —CH₂—CH═CH—C₂H₅, —CH═CH—C₃H₇, —CH₂—CH═CH—CH═CH₂, —CH═CH—CH═CH—CH₃, —CH═CH—CH₂—CH═CH₂, —C(CH₃)═CH—CH═CH₂, —CH═C(CH₃)—CH═CH₂, —CH═CH—C(CH₃)═CH₂, —C₂H₄—C(CH₃)═CH₂, —CH₂—CH(CH₃)—CH═CH₂, —CH(CH₃)—CH₂—CH═CH₂, —CH₂—CH═C(CH₃)₂, —CH₂—C(CH₃)═CH—CH₃, —CH(CH₃)—CH═CH—CH₃, —CH═CH—CH(CH₃)₂, —CH═C(CH₃)—C₂H₅, —C(CH₃)═CH—C₂H₅, —C(CH₃)═C(CH₃)₂, —C(CH₃)₂—CH═CH₂, —CH(CH₃)—C(CH₃)═CH₂, —C(CH₃)═CH—CH═CH₂, —CH═C(CH₃)—CH═CH₂, —CH═CH—C(CH₃)═CH₂, —C₄H₈—CH═CH₂, —C₃H₆—CH═CH—CH₃, —C₂H₄—CH═CH—C₂H₅, —CH₂—CH═CH—C₃H₇, —CH═CH—C₄H₉, —C₃H₆—C(CH₃)═CH₂, —C₂H₄—CH(CH₃)—CH═CH₂, —CH₂—CH(CH₃)—CH₂—CH═CH₂, —CH₂—CH═CH—CH₃, —CH(CH₃)—C₂H₄—CH═CH₂, —C₂H₄—CH═C(CH₃)₂, —C₂H₄—C(CH₃)═CH—CH₃, —CH₂—CH(CH₃)—CH═CH—CH₃, —CH(CH₃)—CH₂—CH═CH—CH₃, —C(C₄H₉)═CH₂, —CH₂—CH═CH—CH(CH₃)₂, —CH₂—CH═C(CH₃)—C₂H₅, —CH₂—C(CH₃)═CH—C₂H₅, —CH(CH₃)—CH═CH—C₂H₅, —CH═CH—CH₂—CH(CH₃)₂, —CH═CH—CH(CH₃)—C₂H₅, —CH═C(CH₃)—C₃H₇, —C(CH₃)═CH—C₃H₇, —CH₂—CH(CH₃)—C(CH₃)═CH₂, —CH(CH₃)—CH₂—C(CH₃)═CH₂, —CH(CH₃)—CH(CH₃)—CH═CH₂, —CH₂—C(CH₃)₂—CH═CH₂, —C(CH₃)₂—CH₂—CH═CH₂, —CH₂—C(CH₃)═C(CH₃)₂, —CH(CH₃)—CH═C(CH₃)₂, —C(CH₃)₂—CH═CH—CH₃, —CH(CH₃)—C(CH₃)═CH—CH₃, —CH═C(CH₃)—CH(CH₃)₂, —C(CH₃)═CH—CH(CH₃)₂, —C(CH₃)═C(CH₃)—C₂H₅, —CH═CH—C(CH₃)₃, —C(CH₃)₂—C(CH₃)═CH₂, —CH(C₂H₅)—C(CH₃)═CH₂, —C(CH₃)(C₂H₅)—CH═CH₂, —CH(CH₃)—C(C₂H₅)═CH₂, —CH₂—C(C₃H₇)═CH₂, —CH₂—C(C₂H₅)═CH—CH₃, —CH(C₂H₅)—CH═CH—CH₃, —C(C₃H₇)═CH—CH₃, —C(C₂H₅)═CH—C₂H₅, —C(C₂H₅)═C(CH₃)₂, —C[C(CH₃)₃]═CH₂, —C[CH(CH₃)(C₂H₅)]═CH₂, —C[CH₂—CH(CH₃)₂]═CH₂, —C₂H₄—CH═CH—CH═CH₂, —CH₂—CH═CH—CH₂—CH═CH₂, —CH═CH—C₂H₄—CH═CH₂, —CH₂—CH═CH—CH═CH—CH₃, —CH═CH—CH₂—CH═CH—CH₃, —CH═CH—CH═CH—C₂H₅, —CH₂—CH═CH—C(CH₃)═CH₂, —CH₂—CH═C(CH₃)—CH═CH₂, —CH₂—C(CH₃)═CH—CH═CH₂, —CH(CH₃)—CH═CH—CH═CH₂, —CH═CH—CH₂—C(CH₃)═CH₂, —CH═CH—CH(CH₃)—CH═CH₂, —CH═C(CH₃)—CH₂—CH═CH₂, —C(CH₃)═CH—CH₂—CH═CH₂, —CH═CH—CH═C(CH₃)₂, —CH═CH—C(CH₃)═CH—CH₃, —CH═C(CH₃)—CH═CH—CH₃, —C(CH₃)═CH—CH═CH—CH₃, —CH═C(CH₃)—C(CH₃)═CH₂, —C(CH₃)═CH—C(CH₃)═CH₂, —C(CH₃)═C(CH₃)—CH═CH₂, —CH═CH—CH═CH—CH═CH₂, —C₅H₁₀—CH═CH₂, —C₄H₈—CH═CH—CH₃, —C₃H₆—CH═CH—C₂H₅, —C₂H₄—CH═CH—C₃H₇, —CH₂—CH═CH—C₄H₉, —C₄H₈—C(CH₃)═CH₂, —C₃H₆—CH(CH₃)—CH═CH₂, —C₂H₄—CH(CH₃)—CH₂—CH═CH₂, —CH₂—CH(CH₃)—C₂H₄—CH═CH₂, —C₃H₆—CH═C(CH₃)₂, —C₃H₆—C(CH₃)═CH—CH₃, —C₂H₄—CH(CH₃)—CH═CH—CH₃, —CH₂—CH(CH₃)—CH₂—CH═CH—CH₃, —C₂H₄—CH═CH—CH(CH₃)₂, —C₂H₄—CH═C(CH₃)—C₂H₅, —C₂H₄—C(CH₃)═CH—C₂H₅, —CH₂—CH(CH₃)—CH═CH—C₂H₅, —CH₂—CH═CH—CH₂—CH(CH₃)₂, —CH₂—CH═CH—CH(CH₃)—C₂H₅, —CH₂—CH═C(CH₃)—C₃H₇, —CH₂—C(CH₃)═CH—C₃H₇, —C₂H₄—CH(CH₃)—C(CH₃)═CH₂, —CH₂—CH(CH₃)—CH₂—C(CH₃)═CH₂, —CH₂—CH(CH₃)—CH(CH₃)—CH═CH₂, —C₂H₄—C(CH₃)₂—CH═CH₂, —CH₂—C(CH₃)₂—CH₂—CH═CH₂, —C₂H₄—C(CH₃)═C(CH₃)₂, —CH₂—CH(CH₃)—CH═C(CH₃)₂, —CH₂—C(CH₃)₂—CH═CH—CH₃, —CH₂—CH(CH₃)—C(CH₃)═CH—CH₃, —CH₂—CH═C(CH₃)—CH(CH₃)₂, —CH₂—C(CH₃)═CH—CH(CH₃)₂, —CH₂—C(CH₃)═C(CH₃)—C₂H₅, —CH₂—CH═CH—C(CH₃)₃, —CH₂—C(CH₃)₂—C(CH₃)═CH₂, —CH₂—CH(C₂H₅)—C(CH₃)═CH₂, —CH₂—C(CH₃)(C₂H₅)—CH═CH₂, —CH₂—CH(CH₃)—C(C₂H₅)═CH₂, —C₂H₄—C(C₃H₇)═CH₂, —C₂H₄—C(C₂H₅)═CH—CH₃, —CH₂—CH(C₂H₅)—CH═CH—CH₃, —CH₂—C(C₄H₉)═CH₂, —CH₂—C(C₃H₇)═CH—CH₃, —CH₂—C(C₂H₅)═CH—C₂H₅, —CH₂—C(C₂H₅)═C(CH₃)₂, —CH₂—C[C(CH₃)₃]═CH₂, —CH₂—C[CH(CH₃)(C₂H₅)]═CH₂, —CH₂—C[CH₂—CH(CH₃)₂]═CH₂, —C₃H₆—CH═CH—CH═CH₂, —C₂H₄—CH═CH—CH₂—CH═CH₂, —CH₂—CH═CH—C₂H₄—CH═CH₂, —C₂H₄—CH═CH—CH═CH—CH₃, —CH₂—CH═CH—CH₂—CH═CH—CH₃, —CH₂—CH═CH—CH═CH—C₂H₅, —C₂H₄—CH═CH—C(CH₃)═CH₂, —C₂H₄—CH═C(CH₃)—CH═CH₂, —C₂H₄—C(CH₃)═CH—CH═CH₂, —CH₂—CH(CH₃)—CH═CH—CH═CH₂, —CH₂—CH═CH—CH₂—C(CH₃)═CH₂, —CH₂—CH═CH—CH(CH₃)—CH═CH₂, —CH₂—CH═C(CH₃)—CH₂—CH═CH₂, —CH₂—C(CH₃)═CH—CH₂—CH═CH₂, —CH₂—CH═CH—CH═C(CH₃)₂, —CH₂—CH═CH—C(CH₃)═CH—CH₃, —CH₂—CH═C(CH₃)—CH═CH—CH₃, —CH₂—C(CH₃)═CH—CH═CH—CH₃, —CH₂—CH═C(CH₃)—C(CH₃)═CH₂, —CH₂—C(CH₃)═CH—C(CH₃)═CH₂, —CH₂—C(CH₃)═C(CH₃)—CH═CH₂, —CH₂—CH═CH—CH═CH—CH═CH₂, —C₆H₁₂—CH═CH₂, —C₅H₁₀—CH═CH—CH₃, —C₄H₈—CH═CH—C₂H₅, C₃H₆—CH═CH—C₃H₇, —C₂H₄—CH═CH—C₄H₉, —C₅H₁₀—C(CH₃)═CH₂, —C₄H₈—CH(CH₃)—CH═CH₂, —C₃H₆—CH(CH₃)—CH₂—CH═CH₂, —C₂H₄—CH(CH₃)—C₂H₄—CH═CH₂, —C₄H₈—CH═C(CH₃)₂, —C₄H₈—C(CH₃)═CH—CH₃, —C₃H₆—CH(CH₃)—CH═CH—CH₃, —C₂H₄—CH(CH₃)—CH₂—CH═CH—CH₃, —C₃H₆—CH═CH—CH(CH₃)₂, —C₃H₆—CH═C(CH₃)—C₂H₅, —C₃H₆—C(CH₃)═CH—C₂H₅, —C₂H₄—CH(CH₃)—CH═CH—C₂H₅, —C₂H₄—CH═CH—CH₂—CH(CH₃)₂, —C₂H₄—CH═CH—CH(CH₃)—C₂H₅, C₂H₄—CH═C(CH₃)—C₃H₇, —C₂H₄—C(CH₃)═CH—C₃H₇, —C₃H₆—CH(CH₃)—C(CH₃)═CH₂, —C₂H₄—CH(CH₃)—CH₂—C(CH₃)═CH₂, —C₂H₄—CH(CH₃)—CH(CH₃)—CH═CH₂, —C₃H₆—C(CH₃)₂—CH═CH₂, —C₂H₄—C(CH₃)₂—CH₂—CH═CH₂, —C₃H₆—C(CH₃)═C(CH₃)₂, —C₂H₄—CH(CH₃)—CH═C(CH₃)₂, —C₂H₄—C(CH₃)₂—CH═CH—CH₃, —C₂H₄—CH(CH₃)—C(CH₃)═CH—CH₃, —C₂H₄—CH═C(CH₃)—CH(CH₃)₂, —C₂H₄—C(CH₃)═CH—CH(CH₃)₂, —C₂H₄—C(CH₃)═C(CH₃)—C₂H₅, —C₂H₄—CH═CH—C(CH₃)₃, —C₂H₄—C(CH₃)₂—C(CH₃)═CH₂, —C₂H₄—CH(C₂H₅)—C(CH₃)═CH₂, —C₂H₄—C(CH₃)(C₂H₅)—CH═CH₂, —C₂H₄—CH(CH₃)—C(C₂H₅)═CH₂, —C₃H₆—C(C₃H₇)═CH₂, —C₃H₆—C(C₂H₅)═CH—CH₃, —C₂H₄—CH(C₂H₅)—CH═CH—CH₃, —C₂H₄—C(C₄H₉)═CH₂, —C₂H₄—C(C₃H₇)═CH—CH₃, —C₂H₄—C(C₂H₅)═CH—C₂H₅, —C₂H₄—C(C₂H₅)═C(CH₃)₂, —C₂H₄—C[C(CH₃)₃]═CH₂, —C₂H₄—C[CH(CH₃)(C₂H₅)]═CH₂, —C₂H₄—C[CH₂—CH(CH₃)₂]═CH₂, —C₄H₈—CH═CH—CH═CH₂, —C₃H₆—CH═CH—CH₂—CH═CH₂, —C₂H₄—CH═CH—C₂H₄—CH═CH₂, —C₃H₆—CH═CH—CH═CH—CH₃, —C₂H₄—CH═CH—CH₂—CH═CH—CH₃, —C₂H₄—CH═CH—CH═CH—C₂H₅, —C₃H₆—CH═CH—C(CH₃)═CH₂, —C₃H₆—CH═C(CH₃)—CH═CH₂, —C₃H₆—C(CH₃)═CH—CH═CH₂, —C₂H₄—CH(CH₃)—CH═CH—CH═CH₂, —C₂H₄—CH═CH—CH₂—C(CH₃)═CH₂, —C₂H₄—CH═CH—CH(CH₃)—CH═CH₂, —C₂H₄—CH═C(CH₃)—CH₂—CH═CH₂, —C₂H₄—C(CH₃)═CH—CH₂—CH═CH₂, —C₂H₄—CH═CH—CH═C(CH₃)₂, —C₂H₄—CH═CH—C(CH₃)═CH—CH₃, —C₂H₄—CH═C(CH₃)—CH═CH—CH₃, —C₂H₄—C(CH₃)═CH—CH═CH—CH₃, —C₂H₄—CH═C(CH₃)—C(CH₃)═CH₂, —C₂H₄—C(CH₃)═CH—C(CH₃)═CH₂, —C₂H₄—C(CH₃)═C(CH₃)—CH═CH₂, —CH═CH-Ph and —C₂H₄—CH═CH—CH═CH—CH═CH₂.

“Alkenylene” refers to a straight or branched chain divalent hydrocarbon radical having 2 to 12 carbon atoms and one or more carbon-carbon double bonds, more preferably from 2 to 8 carbon atoms, and most preferably 2 to 6 carbon atoms.

“Alkynyl” refers to groups of from 2 to 12 carbon atoms, preferably from 2 to 8 carbon atoms, either straight or branched containing at least one triple bond but optionally containing more than one triple bond, and additionally optionally containing one or more double bonded moieties. As used herein, the term “linear or branched C₂-C₈ alkynyl” refers to —C≡CH, —C≡C—CH₃, —CH₂—C≡CH, —C₂H₄—C≡CH, —CH₂—C≡C—CH₃, —C≡C—C₂H₅, —C₂H₄—C≡C—CH₃, —CH₂—C≡C—C₂H₅, —C≡C—C₃H₇, —CH(CH₃)—C≡CH, —CH₂—CH(CH₃)—C≡CH, —CH(CH₃)—CH₂—C≡CH, —CH(CH₃)—C≡C—CH₃, —C₄H₈—C≡CH, —C₃H₆—C≡C—CH₃, —C₂H₄—C≡C—C₂H₅, —CH₂—C≡C—C₃H₇, —C≡C—C₄H₉, —C₂H₄—CH(CH₃)—C≡CH, —CH₂—CH(CH₃)—CH₂—C≡CH, —CH(CH₃)—C₂H₄—C≡CH, —CH₂—CH(CH₃)—C≡C—CH₃, —CH(CH₃)—CH₂—C≡C—CH₃, —CH(CH₃)—C≡C—C₂H₅, —CH₂—C≡C—CH(CH₃)₂, —C≡C—CH(CH₃)—C₂H₅, —C≡C—CH₂—CH(CH₃)₂, —C≡C—C(CH₃)₃, —C₃H₆—C≡CH, —CH(C₂H₅)—C≡C—CH₃, —C(CH₃)₂—C≡C—CH₃, —CH(C₂H₅)—CH₂—C≡CH, —CH₂—CH(C₂H₅)—C≡CH, —C(CH₃)₂—CH₂—C≡CH, —CH₂—C(CH₃)₂—C≡CH, —CH(CH₃)—CH(CH₃)—C≡CH, —CH(C₃H₇)—C≡CH, —C(CH₃)(C₂H₅)—C≡CH, —C≡C—C≡CH, —CH₂—C≡C—C≡CH, —C≡C—C≡C—CH₃, —CH(C≡CH)₂, —C₂H₄—C≡C—C≡CH, —CH₂—C≡C—CH₂—C≡CH, —C≡C—C₂H₄—C≡CH, —CH₂—C≡C—C≡C—CH₃, —C≡C—CH₂—C≡C—CH₃, —C≡C—C≡C—C₂H₅, —C≡C—CH(CH₃)—C≡CH, —CH(CH₃)—C≡C—C≡CH, —CH(C≡CH)—CH₂—C≡CH, —C(C≡CH)₂—CH₃, —CH₂—CH(C≡CH)₂, —CH(C≡CH)—C≡C—CH₃, —C₅H₁₀—C≡CH, —C₄H₈—C≡C—CH₃, —C₃H₆—C≡C—C₂H₅, —C₂H₄—C≡C—C₃H₇, —CH₂—C≡C—C₄H₉, —C₃H₆—CH(CH₃)—C≡CH, —C₂H₄—CH(CH₃)—CH₂—C≡CH, —CH₂—CH(CH₃)—C₂H₄—C≡CH, —C₂H₄—CH(CH₃)—C≡C—CH₃, —CH₂—CH(CH₃)—CH₂—C≡C—CH₃, —CH₂—CH(CH₃)—C≡C—C₂H₅, —C₂H₄—C≡C—CH(CH₃)₂, —CH₂—C≡C—CH(CH₃)—C₂H₅, —CH₂—C≡C—CH₂—CH(CH₃)₂, —CH₂—C≡C—C(CH₃)₃, —CH₂—CH(C₂H₅)—C≡C—CH₃, —CH₂—C(CH₃)₂—C≡C—CH₃, —CH₂—CH(C₂H₅)—CH₂—C≡CH, —C₂H₄—CH(C₂H₅)—C≡CH, —CH₂—C(CH₃)₂—CH₂—C≡CH, —C₂H₄—C(CH₃)₂—C≡CH, —CH₂—CH(CH₃)—CH(CH₃)—C≡CH, —CH₂—CH(C₃H₇)—C≡CH, —CH₂—C(CH₃)(C₂H₅)—C≡CH, —C₃H₆—C≡C—C≡CH, —C₂H₄—C≡C—CH₂—C≡CH, —CH₂—C≡C—C₂H₄—C≡CH, —C₂H₄—C≡C—C≡C—CH₃, —CH₂—C≡C—CH₂—C≡C—CH₃, —CH₂—C≡C—C≡C—C₂H₅, —CH₂—C≡C—CH(CH₃)—C≡CH, —CH₂—CH(CH₃)—C≡C—C≡CH, —CH₂—CH(C≡CH)—CH₂—C≡CH, —CH₂—C(C≡CH)₂—CH₃, —C₂H₄—CH(C≡CH)₂, —CH₂—CH(C≡CH)—C≡C—CH₃, —C₆H₁₂—C≡CH, —C₅H₁₀—C≡C—CH₃, —C₄H₈—C≡C—C₂H₅, —C₃H₆—C≡C—C₃H₇, —C₂H₄—C≡C—C₄H₉, —C₄H₈—CH(CH₃)—C≡CH, —C₃H₆—CH(CH₃)—CH₂—C≡CH, —C₂H₄—CH(CH₃)—C₂H₄—C≡CH, —C₃H₆—CH(CH₃)—C≡C—CH₃, —C₂H₄—CH(CH₃)—CH₂—C≡C—CH₃, —C₂H₄—CH(CH₃)—C≡C—C₂H₅, —C₃H₆—C≡C—CH(CH₃)₂, —C₂H₄—C≡C—CH(CH₃)—C₂H₅, C₂H₄—C≡C—CH₂—CH(CH₃)₂, —C₂H₄—C≡C—C(CH₃)₃, —C₂H₄—CH(C₂H₅)—C≡C—CH₃, —C₂H₄—C(CH₃)₂—C≡C—CH₃, —C₂H₄—CH(C₂H₅)—CH₂—C≡CH, —C₃H₆—CH(C₂H₅)—C≡CH, —C₂H₄—C(CH₃)₂—CH₂—C≡CH, —C₃H₆—C(CH₃)₂—C≡CH, —C₂H₄—CH(CH₃)—CH(CH₃)—C≡CH, —C₂H₄—CH(C₃H₇)—C≡CH, —C₂H₄—C(CH₃)(C₂H₅)—C≡CH, —C₄H₈—C≡C—C≡CH, —C₃H₆—C≡C—CH₂—C≡CH, —C₂H₄—C≡C—C₂H₄—C≡CH, —C₃H₆—C≡C—C≡C—CH₃, —C₂H₄—C≡C—CH₂—C≡C—CH₃, —C₂H₄—C≡C—C≡C—C₂H₅, —C₂H₄—C≡C—CH(CH₃)—C≡CH, —C≡C-Ph, —C₂H₄—CH(CH₃)—C≡C—C≡CH, —C₂H₄—CH(C≡CH)—CH₂—C≡CH, —C₂H₄—C(C≡CH)₂—CH₃, —C₃H₆—CH(C≡CH)₂, and —C₂H₄—CH(C≡CH)—C≡C—CH₃.

“Cycloalkyl” refers to cyclic alkyl groups of from 3 to 12 carbon atoms, preferably from 3 to 8 carbon atoms, having a single cyclic ring or multiple condensed rings which can be optionally substituted with from 1 to 3 alkyl groups. Exemplary cycloalkyl groups include, but are not limited to, single ring structures such as cyclopropyl, cyclobutyl, cyclopentyl, cyclooctyl, 1-methylcyclopropyl, 2-methyl-cyclopentyl, 2-methylcyclooctyl, and the like, or multiple ring structures, including bridged ring systems, such as adamantyl. As used herein, C₃-C₈ cycloalkyl refers to cyclo-C₃H₅, cyclo-C₄H₇, cyclo-C₅H₉, cyclo-C₆H₁₁, cyclo-C₇H₁₃, and cyclo-C₈H₁₅.

“Cycloalkylalkyl” refers to an alkyl substituted with a cycloalkyl, i.e., cycloalkyl-alkyl-groups, preferably having from 1 to 6 carbon atoms in the alkyl moiety and from 3 to 12 carbon atoms in the cycloalkyl moiety. Such cycloalkylalkyl groups are exemplified by cyclopropylmethyl, cyclohexylethyl and the like.

“Aryl” refers to an unsaturated aromatic carbocyclic group of from 6 to 12 carbon atoms inclusively having a single ring (e.g., phenyl) or multiple condensed rings (e.g., naphthyl or anthryl). Exemplary aryls include phenyl, pyridyl, naphthyl and the like.

“Arylalkyl” refers to an alkyl substituted with an aryl, i.e., aryl-alkyl groups, preferably having from 1 to 6 carbon atoms in the alkyl moiety and from 6 to 12 carbon atoms inclusively in the aryl moiety. Such arylalkyl groups are exemplified by benzyl, phenethyl and the like.

“Heteroalkyl, “heteroalkenyl,” and heteroalkynyl,” refer to alkyl, alkenyl and alkynyl as defined herein in which one or more of the carbon atoms are each independently replaced with the same or different heteroatoms or heteroatomic groups. Heteroatoms and/or heteroatomic groups which can replace the carbon atoms include, but are not limited to, —O—, —S—, —S—O—, —NR^{α}—, —PH—, —S(O)—, —S(O)₂—, —S(O)NR^{α}—, —S(O)₂NR^{α}—, and the like, including combinations thereof, where each R^{α} is independently selected from hydrogen, alkyl, heteroalkyl, cycloalkyl, heterocycloalkyl, aryl, and heteroaryl.

“Heteroaryl” refers to an aromatic heterocyclic group of from 1 to 10 carbon atoms and 1 to 4 heteroatoms selected from oxygen, nitrogen and sulfur within the ring. Such heteroaryl groups can have a single ring (e.g., pyridyl or furyl) or multiple condensed rings (e.g., indolizinyl or benzothienyl). As used herein, the term “C₁-C₁ heteroaryl” refers to aromatic residues with one or more heteroatoms such as O, S, N and especially N and refers preferably to

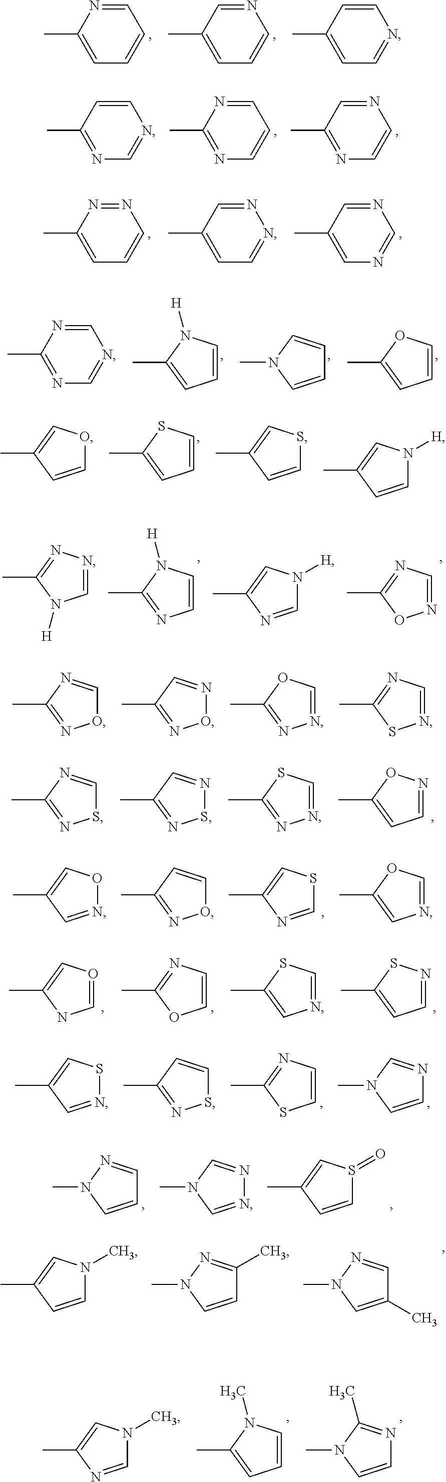

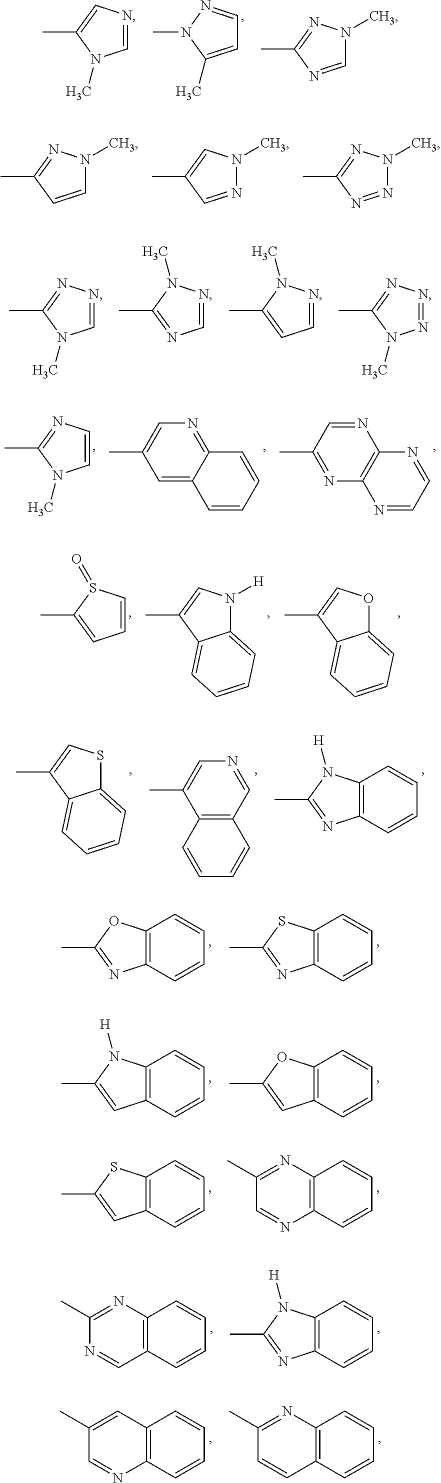

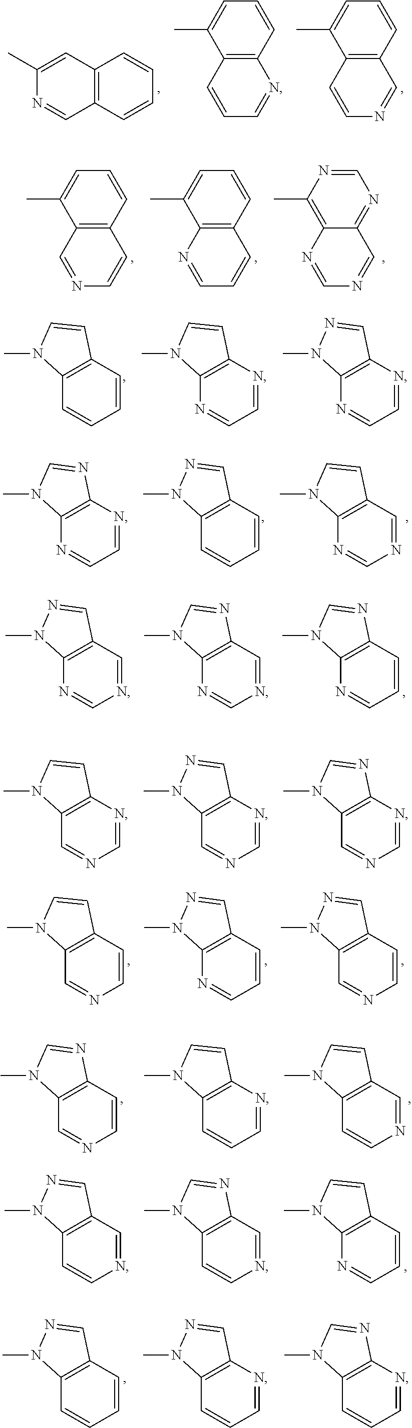

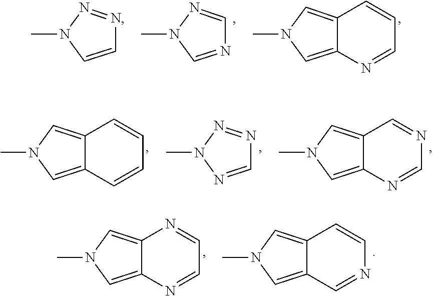

“Heteroarylalkyl” refers to an alkyl substituted with a heteroaryl, i.e., heteroaryl-alkyl-groups, preferably having from 1 to 6 carbon atoms in the alkyl moiety and from 5 to 12 ring atoms inclusively in the heteroaryl moiety. Such heteroarylalkyl groups are exemplified by pyridylmethyl and the like.

“Heterocycle”, “heterocyclic” and interchangeably “heterocycloalkyl” refer to a saturated or unsaturated group having a single ring or multiple condensed rings, from 2 to 9 carbon ring atoms and from 1 to 4 hetero ring atoms inclusively selected from nitrogen, sulfur or oxygen within the ring. Such heterocyclic groups can have a single ring (e.g., piperidinyl or tetrahydrofuryl) or multiple condensed rings (e.g., indolinyl, dihydrobenzofuran or quinuclidinyl). Examples of heterocycles include, but are not limited to, furan, thiophene, thiazole, oxazole, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, pyrrolidine, indoline and the like. As used herein, the term “C₂-C₉ heterocyclyl” covers saturated or partly unsaturated heterocyclic residues with 2 to 9 ring carbon atoms, but not aromatic residues and covers also bicyclic saturated or partly unsaturated residues with 2 to 9 ring carbon atoms, but preferably not fully aromatic residues which are aromatic throughout the bicyclic system but may comprise partly aromatic ring systems, wherein one ring of the bicyclic ring system is aromatic. Preferably “C₂-C₉ heterocyclyl” refers to

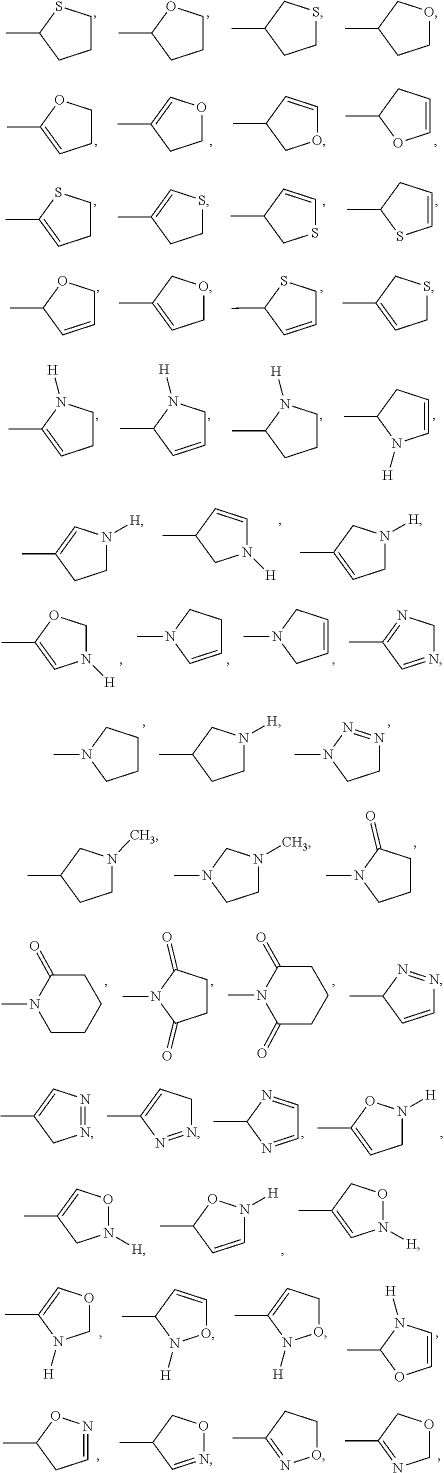

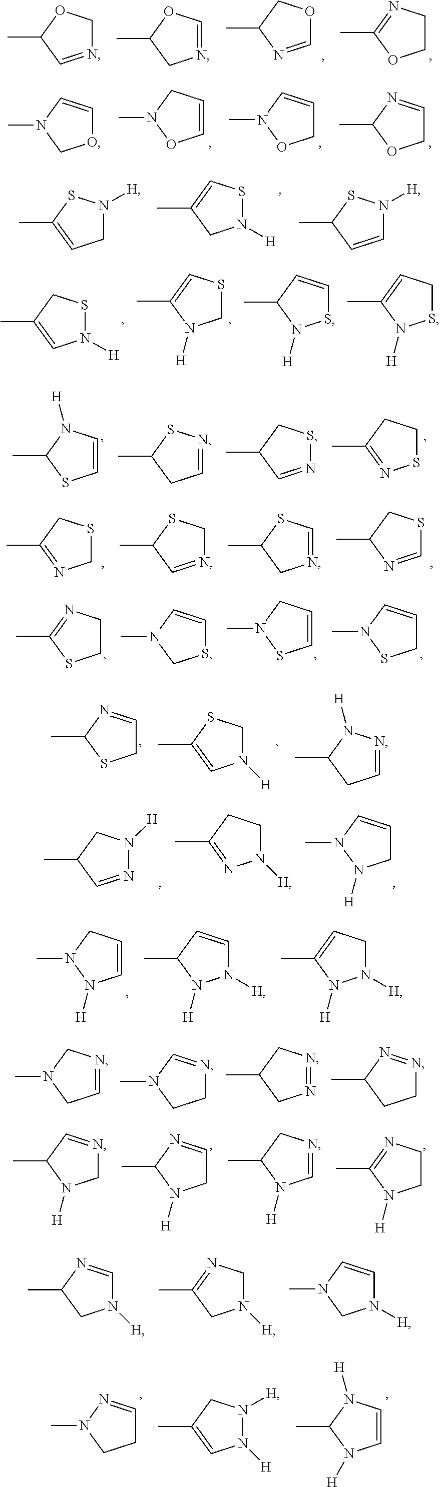

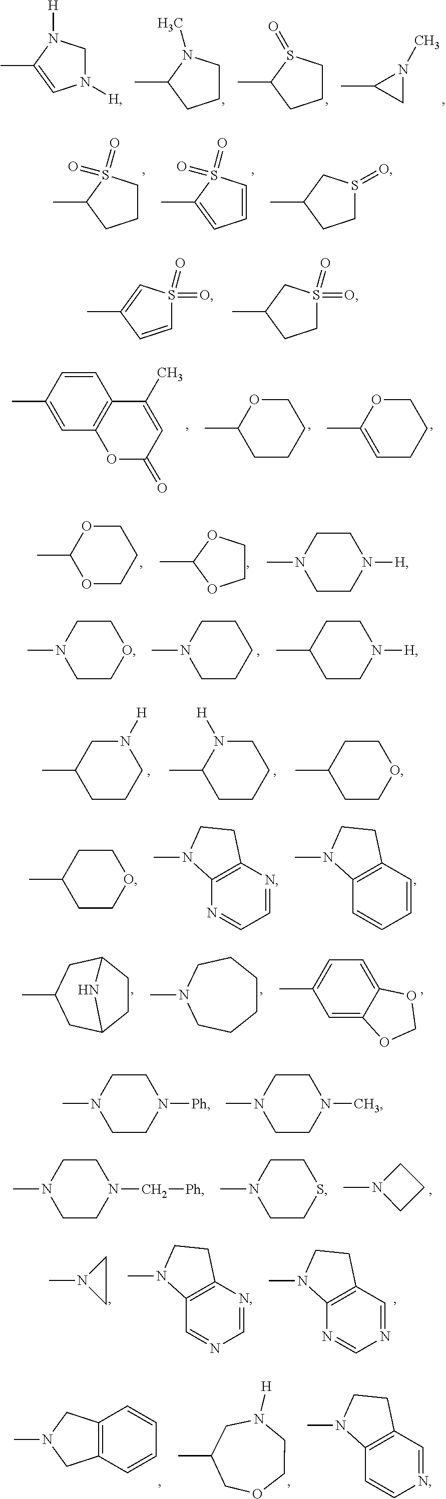

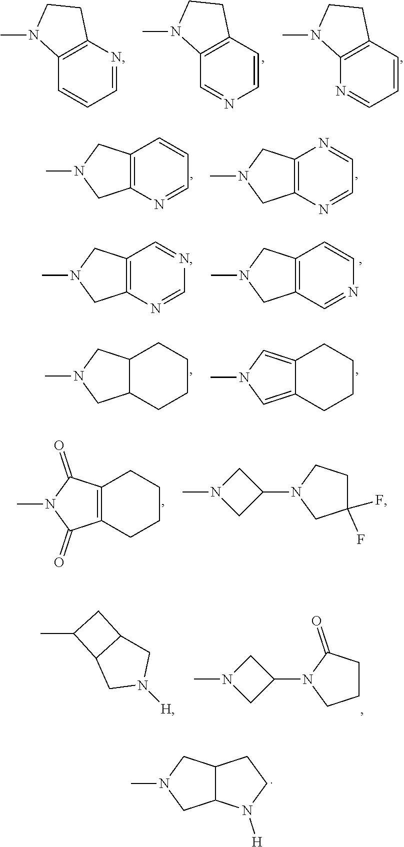

As used herein, the term “3-membered heterocyclyl” refers to a substituted or non substituted ring system of three atoms including at least one heteroatom such as O, S, SO, SO₂, N or NO. The term “4-membered heterocyclyl” refers to a substituted or non substituted ring system of four atoms including at least one heteroatom such as O, S, SO, SO₂, N or NO. The term “5-membered heterocyclyl” refers to a substituted or non substituted ring system of five atoms including at least one heteroatom such as O, S, SO, SO₂, N or NO. The term “6-membered heterocyclyl” refers to a substituted or non substituted ring system of six atoms including at least one heteroatom such as O, S, SO, SO₂, N or NO. The term “monounsaturated 4-membered heterocyclyl” refers to a substituted or non substituted ring system of four atoms including at least one heteroatom such as O, S, SO, SO₂, N or NO, and one double bond. The term “monounsaturated 5-membered heterocyclyl” refers to a substituted or non substituted ring system of five atoms including at least one heteroatom such as O, S, SO, SO₂, N or NO, and one double bond. The term “monounsaturated 6-membered heterocyclyl” refers to a substituted or non substituted ring system of six atoms including at least one heteroatom such as O, S, SO, SO₂, N or NO, and one double bond.

“Heterocycloalkylalkyl” refers to an alkyl substituted with a heterocycloalkyl, i.e., heterocycloalkyl-alkyl groups, preferably having from 1 to 6 carbon atoms in the alkyl moiety and from 3 to 12 ring atoms in the heterocycloalkyl moiety.

“Oxy” refers to a divalent group —O—, which may have various substituents to form different oxy groups, including ethers and esters.

“Alkoxy” or “alkyloxy” are used interchangeably herein to refer to the group —OR^{α}, wherein R^{α} is an alkyl group, including optionally substituted alkyl groups.

“Aryloxy” as used herein refer to the group —OR^{α} wherein R^{α} is an aryl group as defined above including optionally substituted aryl groups as also defined herein.

“Carboxy” refers to —COOH.

“Carboxyalkyl” refers to an alkyl substituted with a carboxy group.

“Carbonyl” refers to the group —C(O)—. Substituted carbonyl refers to the group R^{α}—C(O)—R^{α}, where each R^{α} is independently selected from optionally substituted alkyl, cycloalkyl, cycloheteroalkyl, alkoxy, carboxy, aryl, aryloxy, heteroaryl, heteroarylalkyl, acyl, alkoxycarbonyl, sulfanyl, sulfinyl, sulfonyl, and the like. Typical substituted carbonyl groups including acids, ketones, aldehydes, amides, esters, acyl halides, thioesters, and the like.

“Amino” refers to the group —NH₂. Substituted amino refers to the group —NHR^{α}, NR^{α}R^{α}, and NR^{α}R^{α}R^{α}, where each R^{α} is independently selected from optionally substituted alkyl, cycloalkyl, cycloheteroalkyl, alkoxy, carboxy, aryl, aryloxy, heteroaryl, heteroarylalkyl, acyl, alkoxycarbonyl, sulfanyl, sulfinyl, sulfonyl, and the like. Typical amino groups include, but are limited to, dimethylamino, diethylamino, trimethylammonium, triethylammonium, methylysulfonylamino, furanyl-oxy-sulfamino, and the like.

“Aminoalkyl” refers to an alkyl group in which one or more of the hydrogen atoms are replaced with an amino group, including a substituted amino group.

“Aminocarbonyl” refers to a carbonyl group substituted with an amino group, including a substituted amino group, as defined herein, and includes amides.

“Aminocarbonylalkyl” refers to an alkyl substituted with an aminocarbonyl group, as defined herein.

“Halogen” or “halo” refers to fluoro, chloro, bromo and iodo.

“Haloalkyl” refers to an alkyl group in which one or more of the hydrogen atoms are replaced with a halogen. Thus, the term “haloalkyl” is meant to include monohaloalkyls, dihaloalkyls, trihaloalkyls, etc. up to perhaloalkyls. As used herein, the expression “C₁-C₂ haloalkyl” includes 1-fluoromethyl, difluoromethyl, trifluoromethyl, 1-fluoroethyl, 1,1-difluoroethyl, 1,2-difluoroethyl, 1,1,1 trifluoroethyl, perfluoroethyl, etc.

“Hydroxy” refers to —OH.

“Hydroxyalkyl” refers to an alkyl substituted with one or more hydroxy group.

“Thio” or “sulfanyl” refers to —SH. Substituted thio or sulfanyl refers to —S—R^{α}, where R^{α} is an alkyl, aryl or other suitable substituent.

“Alkylthio” refers to —SR^{α}, where R^{α} is an alkyl, which can be optionally substituted. Typical alkylthio group include, but are not limited to, methylthio, ethylthio, n-propylthio, and the like.

“Alkylthioalkyl” refers to an alkyl substituted with an alkylthio group, —SR^{α}, where R^{α} is an alkyl, which can be optionally substituted.

“Thiocarbonyl” refers to a carbonyl group substituted with a thio group, including a substituted thio group, as defined herein, and includes thiosesters.

“Sulfonyl” refers to —SO₂—. Substituted sulfonyl refers to —SO₂—R^{α}, where R^{α} is an alkyl, aryl or other suitable substituent.

“Alkylsulfonyl” refers to —SO₂—R^{α}, where R^{α} is an alkyl, which can be optionally substituted. Typical alkylsulfonyl groups include, but are not limited to, methylsulfonyl, ethylsulfonyl, n-propylsulfonyl, and the like.

“Alkylsulfonylalkyl” refers to an alkyl substituted with an alkylsulfonyl group, —SO₂—R^{α}, where R^{α} is an alkyl, which can be optionally substituted.

“Membered ring” is meant to embrace any carbocyclic or heterocyclic, aromatic or non aromatic structure. The number preceding the term “membered” denotes the number of skeletal atoms that constitute the ring. Thus, for example, cyclohexyl, pyridine, pyran and thiopyran are 6-membered rings and cyclopentyl, pyrrole, furan, and thiophene are 5-membered rings.

“Fused bicyclic ring” refers to both unsubstituted and substituted carbocyclic and/or heterocyclic ring moieties having 5 or 8 atoms in each ring, the rings having 2 common atoms.

“Optionally substituted” as used herein with respect to the foregoing chemical groups means that positions of the chemical group occupied by hydrogen can be substituted with another atom, such as carbon, oxygen, nitrogen, or sulfur, or a chemical group, exemplified by, but not limited to, hydroxy, oxo, nitro, methoxy, ethoxy, alkoxy, substituted alkoxy, trifluoromethoxy, haloalkoxy, fluoro, chloro, bromo, iodo, halo, methyl, ethyl, propyl, butyl, alkyl, alkenyl, alkynyl, substituted alkyl, trifluoromethyl, haloalkyl, hydroxyalkyl, alkoxyalkyl, thio, alkylthio, acyl, carboxy, alkoxycarbonyl, carboxamido, substituted carboxamido, alkylsulfonyl, alkylsulfinyl, alkylsulfonylamino, sulfonamido, substituted sulfonamido, cyano, amino, substituted amino, alkylamino, dialkylamino, aminoalkyl, acylamino, amidino, amidoximo, hydroxamoyl, phenyl, aryl, substituted aryl, aryloxy, arylalkyl, arylalkenyl, arylalkynyl, pyridyl, imidazolyl, heteroaryl, substituted heteroaryl, heteroaryloxy, heteroarylalkyl, heteroarylalkenyl, heteroarylalkynyl, cyclopropyl, cyclobutyl, cyclopentyl, cyclohexyl, cycloalkyl, cycloalkenyl, cycloalkylalkyl, substituted cycloalkyl, cycloalkyloxy, pyrrolidinyl, piperidinyl, morpholino, heterocycle, (heterocycle)oxy, and (heterocyclyl)alkyl; where preferred heteroatoms are oxygen, nitrogen, and sulfur. However it is clear to a skilled person that the term “can be substituted” refers to the replacement of a hydrogen atom by one of the above-mentioned chemical groups. Additionally, where open valences exist on these substitute chemical groups they can be further substituted with alkyl, cycloalkyl, aryl, heteroaryl, and/or heterocycle groups, that where these open valences exist on carbon they can be further substituted by halogen and by oxygen-, nitrogen-, or sulfur-bonded substituents, and where multiple such open valences exist, these groups can be joined to form a ring, either by direct formation of a bond or by formation of bonds to a new heteroatom, preferably oxygen, nitrogen, or sulfur. It is further contemplated that the above substitutions can be made provided that replacing the hydrogen with the substituent does not introduce unacceptable instability to the molecules of the present disclosure, and is otherwise chemically reasonable. One of ordinary skill in the art would understand that with respect to any chemical group described as optionally substituted, only sterically practical and/or synthetically feasible chemical groups are meant to be included. Finally, “optionally substituted” as used herein refers to all subsequent modifiers in a term or series of chemical groups. For example, in the term “optionally substituted arylalkyl,” the “alkyl” portion and the “aryl” portion of the molecule may or may not be substituted, and for the series “optionally substituted alkyl, alkenyl, alkynyl, alkoxy, carboxy, aminocarbonyl, thiocarbonyl, heteroalkyl, heteroalkenyl, heteroalkynyl, carboxyalkyl, aminoalkyl, haloalkyl, alkylthioalkyl, cycloalkyl, aryl, arylalkyl, heterocycloalkyl, heteroaryl, and heteroarylalkyl,” alkyl, alkenyl, alkynyl, alkoxy, carboxy, aminocarbonyl, thiocarbonyl, heteroalkyl, heteroalkenyl, heteroalkynyl, carboxyalkyl, aminoalkyl, haloalkyl, alkylthioalkyl, cycloalkyl, aryl, arylalkyl, heterocycloalkyl, heteroaryl, and heteroarylalkyl groups, independently of the others, may or may not be substituted.

Preferably “optionally substituted” means that 1 to 4 positions of the chemical group occupied by hydrogen can be substituted by Z^{a}, Z^{b}, Z^{c} and Z^{d}, wherein Z^{a} to Z^{d} represent independently of each other —CH₃, —C₂H₅, —C₃H₇, —CH(CH₃)₂, —OH, —OCH₃, —OC₂H₅, —OC₃H₇, —NH₂, —N(CH₃)₂, —F, —Cl, —Br, —I, —CN, —CH₂F, —CHF₂, —CF₃, —OCHF₂, or —OCF₃.

Preferably “optionally substituted” means that 1 to 5 positions of the chemical group occupied by hydrogen can be substituted by Z^{a}, Z^{b}, Z^{c} Z^{d} and Z^{e}, wherein Z^{a} to Z^{e} represent independently of each other —CH₃, —C₂H₅, —C₃H₇, —CH(CH₃)₂, —OH, —OCH₃, —OC₂H₅, —OC₃H₇, —NH₂, —N(CH₃)₂, —F, —Cl, —Br, —I, —CN, —CH₂F, —CHF₂, —CF₃, —OCHF₂, or —OCF₃.

### Polypeptides Having Enzyme Activity to Reduce Imines

As shown in FIG. 1, in the BHAP, the enzyme β-hydroxyaspartate aldolase (2) catalyzes the condensation of glycine and glyoxylate to (2R,3S)-β-hydroxyaspartate and the enzyme β-hydroxyaspartate dehydratase (3) catalyzes the subsequent dehydration to iminosuccinate. The iminosuccinate is reduced to aspartate by the iminosuccinate reductase (4) in the presence of the cofactor NADH and the formed aspartate is finally converted with glyoxylate to oxaloacetate in the presence of aspartate-glyoxylate aminotransferase (1).

It was generally assumed that the product of the dehydration of (2R,3S)-β-hydroxyaspartate is oxaloacetate (Biochem. J. 1965, 97(2), 547). However, the inventors could show that the reaction product of the β-hydroxyaspartate dehydratase enzyme is actually iminosuccinate, a compound that is highly labile in aqueous solution and hydrolyzes to oxaloacetate (t_{1/2}=1.48 s; calculated from data of Mortarino et al., 1996, Eur. J. Biochem. 239(2), 418-426). To this end, glyoxylate, glycine and the required cofactors together with the β-hydroxyaspartate aldolase and dehydratase enzymes were brought to reaction in D₂O and subsequently reduced to monodeuterated aspartate by sodium cyanoborohydride (FIG. 3). The conversion to monodeuterated aspartate upon addition of sodium cyanoborohydride demonstrates the formation of iminosuccinate by the β-hydroxyaspartate dehydratase enzyme catalyzed dehydration of β-hydroxyaspartate.

By phylogenetic analysis the inventors have revealed that the BHAP is widely distributed among proteobacteria, particularly alpha- and gamma-proteobacteria. Thus, proteins, which exhibit imine reductase and/or iminosuccinate reductase activity, are widespread in many proteobacteria, such as Aestuariivita boseongensis, Agrobacterium sp., Ahrensia sp., Aminobacter aminovorans, Amphritea atlantica, Antarctobacter heliothermus, Aquisalimonas asiatica, Aurantimonas altamirensis, Aureimonas altamirensis, Brevirhabdus pacifica, Citreicella marina, Citreicella sp., Citreicella thiooxidans, Citreimonas salinaria, Colwellia piezophila, Colwellia psychrerythraea, Colwellia sp, Cribrihabitans marinus, Defluviimonas indica, Defluviimonas sp., Dinoroseobacter shibae, Ensifer fredii, Ensifer meliloti, Ensifer sp., Glaciecola sp., Granulosicoccus antarcticus, Halocynthiibacter sp., Hasllibacter halocynthiae, Hyphomicrobium sulfonivorans, Jannaschia pohangensis, Jannaschia rubra, Jannaschia sp., Labrenzia aggregata, Labrenzia alba, Labrenzia alexandrii, Labrenzia sp., Leisingera aquaemixtae, Leisingera nanhaiensis, Leisingera sp., Litoreibacter ascidiaceicola, Litoreibacter halocynthiae, Litoreibacter janthinus, Litoreibacter meonggei, Litoreibacter ponti, Loktanella koreensis, Loktanella litorea, Loktanella maricola, Loktanella rosea, Loktanella sediminilitoris, Loktanella sediminum, Loktanella sp., Loktanella vestfoldensis, Mameliella alba, Maribius sp., Marinobacter psychrophilus, Marinobacter sp., Marinobacterium lutimaris, Marinobacterium mangrovicola, Marinobacterium sp., Marinomonas sp., Marinovum algicola, Maritimibacter sp., Marivita geojedonensis, Marivita hallyeonensis, Mesorhizobium sp., Mesorhizobium sp., Methylobacterium komagatae, Methylobacterium mesophilicum, Methylobacterium radiotolerans, Methylobacterium sp., Methylobacterium sp., Methylopila sp., Neptunomonas antarctica, Nitratireductor sp., Oceanicola flagellatus, Oceanicola nitratireducens, Oceanicola sp., Oceaniovalibus guishaninsula, Octadecabacter antarcticus, Octadecabacter arcticus, Octadecabacter temperatus, Palleronia marisminoris, pAqui_F126, Paracoccus alcaliphilus, Paracoccus alkenifer, Paracoccus aminophilus, Paracoccus aminovorans, Paracoccus denitrificans, Paracoccus halophilus, Paracoccus homiensis, Paracoccus isoporae, Paracoccus pantotrophus, Paracoccus saliphilus, Paracoccus sediminis, Paracoccus sp., Paracoccus thiocyanatus, Paracoccus versutus, Paracoccus yeei, Pararhodobacter aggregans, pCaer_C109, pDaep_A276, Pelagibaca bermudensis, Pelagicola litoralis, Pelagimonas varians, Phaeobacter gallaeciensis, Phaeobacter inhibens, Planktotalea frisia, pMeth_A285, Ponticoccus litoralis, Ponticoccus sp., Poseidonocella pacifica, Pseudomonas stutzeri, Pseudopelagicola gijangensis, Pseudorhodobacter antarcticus, Pseudoruegeria haliotis, Pseudoruegeria marinistellae, Psychrobacter arcticus, Psychrobacter cryohalolentis, Psychrobacter sp., Psychrobacter urativorans, Puniceibacterium sediminis, Rhizobium etli, Rhizobium etli, Rhizobium etli, Rhizobium leguminosarum, Rhizobium lusitanum, Rhizobium rhizogenes, Rhizobium sp., Rhizobium taibaishanense, Rhizobium tropici, Rhizobium yanglingense, Rhodobaca barguzinensis, Rhodobacteraceae bacterium, Rhodobacteraceae sp., Rhodobacterales bacterium, Rhodovibrio salinarum, Rhodovulum kholense, Rhodovulum sp., Rhodovulum sulfidophilum, Roseinatronobacter thiooxidans, Roseivivax halodurans, Roseivivax isoporae, Roseivivax lentus, Roseivivax sediminis, Roseobacter denitrificans, Roseobacter litoralis, Roseobacter sp., Roseovarius azorensis, Roseovarius indicus, Roseovarius litoreus, Roseovarius lutimaris, Roseovarius marisflavi, Roseovarius mucosus, Roseovarius nubinhibens, Roseovarius sediminilitoris, Roseovarius sp., Roseovarius tolerans, Rubellimicrobium mesophilum, Rubrimonas cliftonensis, Ruegeria atlantica, Ruegeria conchae, Ruegeria faecimaris, Ruegeria halocynthiae, Ruegeria marina, Ruegeria mobilis, Ruegeria scottomollicae, Ruegeria sp., Sagittula stellata, Salinihabitans flavidus, Shimia haliotis, Shimia sagamensis, Silicibacter sp., Sinorhizobium americanum, Sinorhizobium fredii, Sinorhizobium terangae, Solemya velum, Sphingomonas sp., Stappia aggregata, Starkeya novella, Sulfitobacter pseudonitzschiae, Sulfitobacter sp., Tateyamaria omphalii, Tateyamaria sp., Thalassobacter sp., Thalassobacter stenotrophicus, Thalassobius abyssi, Thalassobius aestuarii, Thalassobius mediterraneus, Thalassotalea sp., uncultured Rhodobacteriaceae, Yangia pacifica, Yangia pacifica, Yangia sp., Paracoccus sulfuroxidans, AP Rhodobacteraceae bacterium and Silicibacter pomeroyi.

Thus, the present invention is also directed to a method of preparing a primary amine compound of general formula (IB), comprising the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises the conserved amino acid sequence of GXKXGX₈GXKXGGX₂PXNX₇NHQSX₃LFX₄GX₈NX₂TAXRTAAX₄SX₃LX₈GX₂GAGXQ X₃QX₁₅WNX₃₉SX₁₅HX₃MGTDTX₂KXEX₁₃DX₃QX₄GEXQX₁₆GX₉RX₆TX₂DGXGX₃QDX A (SEQ ID NO: 610), wherein X represents independently for each occurrence an amino acid, wherein R¹ and R² have the meanings as defined herein, and
   the polypeptide is an imine reductase selected from Aestuariivita boseongensis, Agrobacterium sp., Ahrensia sp., Aminobacter aminovorans, Amphritea atlantica, Antarctobacter heliothermus, Aquisalimonas asiatica, Aurantimonas altamirensis, Aureimonas altamirensis, Brevirhabdus pacifica, Citreicella marina, Citreicella sp., Citreicella thiooxidans, Citreimonas salinaria, Colwellia piezophila, Colwellia psychrerythraea, Colwellia sp, Cribrihabitans marinus, Defluviimonas indica, Defluviimonas sp., Dinoroseobacter shibae, Ensifer fredii, Ensifer meliloti, Ensifer sp., Glaciecola sp., Granulosicoccus antarcticus, Halocynthiibacter sp., Hasllibacter halocynthiae, Hyphomicrobium sulfonivorans, Jannaschia pohangensis, Jannaschia rubra, Jannaschia sp., Labrenzia aggregata, Labrenzia alba, Labrenzia alexandrii, Labrenzia sp., Leisingera aquaemixtae, Leisingera nanhaiensis, Leisingera sp., Litoreibacter ascidiaceicola, Litoreibacter halocynthiae, Litoreibacter janthinus, Litoreibacter meonggei, Litoreibacter ponti, Loktanella koreensis, Loktanella litorea, Loktanella maricola, Loktanella rosea, Loktanella sediminilitoris, Loktanella sediminum, Loktanella sp., Loktanella vestfoldensis, Mameliella alba, Maribius sp., Marinobacter psychrophilus, Marinobacter sp., Marinobacterium lutimaris, Marinobacterium mangrovicola, Marinobacterium sp., Marinomonas sp., Marinovum algicola, Maritimibacter sp., Marivita geojedonensis, Marivita hallyeonensis, Mesorhizobium sp., Mesorhizobium sp., Methylobacterium komagatae, Methylobacterium mesophilicum, Methylobacterium radiotolerans, Methylobacterium sp., Methylobacterium sp., Methylopila sp., Neptunomonas antarctica, Nitratireductor sp., Oceanicola flagellatus, Oceanicola nitratireducens, Oceanicola sp., Oceaniovalibus guishaninsula, Octadecabacter antarcticus, Octadecabacter arcticus, Octadecabacter temperatus, Palleronia marisminoris, pAqui_F126, Paracoccus alcaliphilus, Paracoccus alkenifer, Paracoccus aminophilus, Paracoccus aminovorans, Paracoccus denitrificans, Paracoccus halophilus, Paracoccus homiensis, Paracoccus isoporae, Paracoccus pantotrophus, Paracoccus saliphilus, Paracoccus sediminis, Paracoccus sp., Paracoccus thiocyanatus, Paracoccus versutus, Paracoccus yeei, Pararhodobacter aggregans, pCaer_C109, pDaep_A276, Pelagibaca bermudensis, Pelagicola litoralis, Pelagimonas varians, Phaeobacter gallaeciensis, Phaeobacter inhibens, Planktotalea frisia, pMeth_A285, Ponticoccus litoralis, Ponticoccus sp., Poseidonocella pacifica, Pseudomonas stutzeri, Pseudopelagicola gijangensis, Pseudorhodobacter antarcticus, Pseudoruegeria haliotis, Pseudoruegeria marinistellae, Psychrobacter arcticus, Psychrobacter cryohalolentis, Psychrobacter sp., Psychrobacter urativorans, Puniceibacterium sediminis, Rhizobium etli, Rhizobium etli, Rhizobium etli, Rhizobium leguminosarum, Rhizobium lusitanum, Rhizobium rhizogenes, Rhizobium sp., Rhizobium taibaishanense, Rhizobium tropici, Rhizobium yanglingense, Rhodobaca barguzinensis, Rhodobacteraceae bacterium, Rhodobacteraceae sp., Rhodobacterales bacterium, Rhodovibrio salinarum, Rhodovulum kholense, Rhodovulum sp., Rhodovulum sulfidophilum, Roseinatronobacter thiooxidans, Roseivivax halodurans, Roseivivax isoporae, Roseivivax lentus, Roseivivax sediminis, Roseobacter denitrificans, Roseobacter litoralis, Roseobacter sp., Roseovarius azorensis, Roseovarius indicus, Roseovarius litoreus, Roseovarius lutimaris, Roseovarius marisflavi, Roseovarius mucosus, Roseovarius nubinhibens, Roseovarius sediminilitoris, Roseovarius sp., Roseovarius tolerans, Rubellimicrobium mesophilum, Rubrimonas cliftonensis, Ruegeria atlantica, Ruegeria conchae, Ruegeria faecimaris, Ruegeria halocynthiae, Ruegeria marina, Ruegeria mobilis, Ruegeria scottomollicae, Ruegeria sp., Sagittula stellata, Salinihabitans flavidus, Shimia haliotis, Shimia sagamensis, Silicibacter sp., Sinorhizobium americanum, Sinorhizobium fredii, Sinorhizobium terangae, Solemya velum, Sphingomonas sp., Stappia aggregata, Starkeya novella, Sulfitobacter pseudonitzschiae, Sulfitobacter sp., Tateyamaria omphalii, Tateyamaria sp., Thalassobacter sp., Thalassobacter stenotrophicus, Thalassobius abyssi, Thalassobius aestuarii, Thalassobius mediterraneus, Thalassotalea sp., uncultured Rhodobacteriaceae, Yangia pacifica, Yangia pacifica, Yangia sp., Paracoccus sulfuroxidans, AP Rhodobacteraceae bacterium or Silicibacter pomeroyi.

The imine reductases identified by the inventors comprise the conserved amino amino acids among the proteobacteria as listed in Table 1. The conserved amino acid sequence of SEQ ID NO: 610 reflects these conserved sites. The conserved sequence—in its common meaning in the art—is determined from multiple sequence alignments of the imine reductase sequences, wherein the sites conserved throughout all aligned sequences are listed in Table 1. As the aligned imine reductase sequences may contain gap(s) or deletion(s), the conserved sequence determined from these aligned sequences may also contain gap(s) or deletion(s). Although not explicitly mentioned throughout the disclosure, a skilled person readily envisions that the conserved sequence comprises gap(s) or deletion(s) which were generated by the sequence alignment.

TABLE 1
Conserved sites of the imine reductase enzyme
in proteobacteria identified by the inventors.

| | amino acid |
|---|---|
| position | sequence |
| 79 | G |
| 81 | K |
| 83 | G |
| 92 | G |
| 94 | K |
| 96-97 | GG |
| 100 | P |
| 102 | N |
| 110-113 | NHQS |
| 117-118 | LF |
| 123 | G |
| 132 | N |
| 135-136 | TA |
| 138-141 | RTAA |
| 146 | S |
| 150 | L |
| 159 | G |
| 162-164 | GAG |
| 166 | Q |
| 170 | Q |
| 186-187 | WN |
| 227 | S |
| 243 | H |
| 247-251 | MGTDT |
| 254 | K |
| 256 | E |
| 270 | D |
| 274 | Q |
| 279-280 | GE |
| 282 | Q |
| 299 | G |
| 309 | R |
| 316 | T |
| 319-320 | DG |
| 323 | G |
| 326-327 | QD |
| 329 | A |

Similarly, conserved amino acid sequences of SEQ ID NO 611 to 621 cover a subset of the imine reductases SEQ ID NO 300-598 identified by the inventors.

TABLE 2
Amino acid sequences of the conserved amino acid sequences of SEQ ID
NO 611 to 621, wherein each occurrence of X represents an amino acid or a gap.

| SEQ | |
|---|---|
| ID | |
| NO | amino acid sequence |
| 611 | GXKXGXXXXXXXXGXKXGGXXPXNXXXXXXXNHQSXXXLFXXXXGXXXXXXXXN |
| | XXTAXRTAAXXXXSXXXLXXXXXXXXGXXGAGXQXXXQXXXXXXXXXXXXXXXWN |
| | XXXXXXXXXXXXXXXXXXXXXXXXXXXXXXXXXXXXXXXSXXXXXXXXXXXXXXXH |
| | XXXMGTDTXXKXEXXXXXXXXXXXXXDXXXQXXXXGEXQXXXXXXXXXXXXXXXX |
| | GXXXXXXXXXRXXXXXXTXXDGXGXXXQDXA |
| 612 | MXXXXAXNFPVXREXIGXXXALYGFKXGFDXXXXXLGXKXGGXWPXNXXXXXXNH |
| | QSXXXLFXXXXGXXXXXVGGNXLTAXRTAAXXXXSIXXLAXXXXXXXGXXGAGXQX |
| | XFQXXAAXXXXXXEXXXXWNXXXXXXXXXXXXXXXXXXPFXXVXXXXXXXXXDVX |
| | XXITSXXXXXXXXXXXXXXTHXAXMGTDTXGKQEXXXXXXXXAXXFTDXXXQXXXX |
| | GEXQHAXXXXXXXXXXXXXXGXVXXXXXXXRXXXXXXTXXDGXGVXLQDLAVAXX |
| | XXXXA |
| 613 | EXXXXXXXXXXXXFXAXEXVFXXMAXXXAXNFPVVREXIGHXXALYGFKXGFDXXX |
| | XXLGXKXGGXWPXNXXXXXXNHQSTVXLFDXXXGXXXAXVGGNXLTAXRTAAXSA |
| | VSIXXLAXXXAKXLGXXGAGXQXXFQXXAAXXXXXXEXVXXWNXXPXXLXXLXXXX |
| | XXXXXPFEXVXXXXLXXXXDVIXXITSXXXXXLXXXXXXXXTHXAXMGTDTXGKQEX |
| | XXXXXXXAXXFTDXXXQXXXIGEXQHAXXXXXXXXXXXXXXGXVXXXXXXXRXXXX |
| | XXTXXDGXGVGLQDLAVAXXXXXXA |
| 614 | MAXXXAXNFPVXREAIGXXDALYGFKXGFDXXXXXLGXKXGGXWPXNXXXXLXNH |
| | QSXXXLFDXDXGXXXXXVGGNXLTAXRTAAXXXXSIXXLAXXXAXVXGMIGAGHQX |
| | XFQXRAAXXXXXFEXXXXWNXHPXXXXXXXXXXXEXXXPFXXVXXXXXXXEXDVX |
| | XXITSXFXXXXXXXXXXXXTHXAXMGTDTXGKQEXXXXLXXXAXXFTDEXAQXXXX |
| | GEXQHAXXXXXXXXXXXXXXGXVXXGXXXGRXXXXXXTXFDGTGVXLQDLAVAXX |
| | XVXXA |
| 615 | MXXXXAXNFPVXREXIGXXXALYGFKXGFDXXXXXLGXKXGGXWPXNXXXXXXNH |
| | QSXXFLFXXDXGXXXAXVGGNXLTAXRTAAXSXXSIXXLAXXXXKVXGMXGAGHQ |
| | XXFQXXAAXXXXXFEKXXXWNXXXXXLXXLXXXAXXXXXPFXXVXXXXXXXXXDVI |
| | XXITSXFXXXXXXXXXXXXTHXAXMGTDTXGKQEXXXXLXXXAXXFTDEXXQXXXI |
| | GEXQHAXXXXXXXXXXXXXXGXVXXGXXXGRXXXXXXTXXDGXGVXLQDLAVAX |
| | XXXXXA |
| 616 | MXXXXAXNFPVXREAIGXXDALYGFKXGFDXXXXXLGXKXGGXWPXNXXXXXXNH |
| | QSXXXLFXXDXGXXXXXVGGNXLTAXRTAAXSXXSIXXLAXXXXKVXGMXGAGHQ |
| | XXFQXRAAXXXXXFEKXXXWNXXXXXXXXLXXXXXEXXXPFXXVXXXXXXXXXDVI |
| | XXITSXFXXXXXXXXXXXXTHXAXMGTDTXGKQEXXXXLXXXAXXFTDEXAQXXXI |
| | GEXQHAXXXXXXXXXXXXXXGXVXXGXXXGRXXXXXXTXFDGTGVXLQDLAVAXX |
| | XXXXA |
| 617 | MXXVXEXXIXGLMXPXAAFXAXEXXFAXMXXXXAXNFPVVREAXGXXDALYGFKG |
| | GFDXXXXXLGLKAGGYWPXNXXXXXINHQSTXFLFDXDXGRXXAAXGGNLLTALR |
| | TAAASAVSXKXLAPXGAXVLGMIGAGHQSXFQMXAXXXXXXFXXVXGWNPHPXML |
| | XRLXXTAXXLGLPFEAVXLXXLGXXADVIXXITSXFXXLLXXXHVXGXTHXAAMGTD |
| | TKGKQELDXXLVXRXXXXTDEXAQXXXIGEXQHXXAXXXXXXXXXXXXGXXXXGX |
| | XXGRXXXXXTXFDGTGVGLQDLAVAXXXXXXAXXXGXA |
| 618 | MXVVXEKXIAGLMXPXAAFEAIEAXFAXMARRXAXNFPVVREAIGXXDALYGFKGG |
| | FDXXXLXLGLKAGGYWPXNQXXXLINHQSTVFLFDPDXGRXXAAXGGNLLTALRTA |
| | AASAVSIKXLAPXGAKVLGMIGAGHQSXFQMRAXAXVHXFEKVIGWNPHPEMLXRL |
| | XXTAXXLGLPFEAVXLXXLGXEADVIXSITSXFXXLLXXXHVKGPTHXAAMGTDTKG |
| | KQELDXXLVXRARXXTDEXAQSXXIGEXQHAXAXXLIXXXXXXEXGAVVXGXXXGR |
| | XXAEVTXFDGTGVGLQDLAVAXXXXEXAKXXGXAXXVE |
| 619 | MXVVAEKEIAGLMTPEAAFEAIEAXFASMARRKAYNFPVVREAIGHEDALYGFKGG |
| | FDAXALXLGLKAGGYWPXNQKHXLINHQSTVFLFDPDTGRXXAAXGGNLLTALRTA |
| | AASAVSIKXLAPXGAKVLGMIGAGHQSAFQMRAXAXVHXFEKVIGWNPHPEMLXRL |
| | AXTAAXLGLPFEAVXLXRLGXEADVIXSITSSFSPLLXXXHVKGPTHXAAMGTDTKG |
| | KQELDPALVARARXFTDEVAQSVXIGECQHAIAXXLIXEXXXGEXGAVVXGDXPGR |
| | XXAEVTXFDGTGVGLQDLAVAXXVXEXAKXXGXAQXVEI |
| 620 | MLVVAEKEIAGLMTPEAAFEAIEAVFASMARRKAYNFPVVREAIGHEDALYGFKGG |
| | FDAXALXLGLKAGGYWPXNQKHXLINHQSTVFLFDPDTGRVSAAVGGNLLTALRTA |
| | AASAVSIKXLAPKGAKVLGMIGAGHQSAFQMRAAANVHRFEKVIGWNPHPEMLXR |
| | LADTAAELGLPFEAVXLXRLGXEADVIXSITSSFSPLLXXXHVKGPTHXAAMGTDTK |
| | GKQELDPALVARARXFTDEVAQSVSIGECQHAIAXXLIXEXQXGEXGAVVAGDXPG |
| | RGDAEVTXFDGTGVGLQDLAVAXXVXEXAKXXGXAQEVEI |
| 621 | MLVVAEKEIAGLMTPEAAFEAlEAVFASMARRKAYNFPVVREAIGHEDALYGFKGG |
| | FDASALXLGLKAGGYWPNNQKHNLINHQSTVFLFDPDTGRVSAAVGGNLLTALRTA |
| | AASAVSIKYLAPKGAKVLGMIGAGHQSAFQMRAAANVHRFEKVIGWNPHPEMLSR |
| | LADTAAELGLPFEAVELDRLGXEADVIXSITSSFSPLLMXXHVKGPTHXAAMGTDTK |
| | GKQELDPALVARARXFTDEVAQSVSIGECQHAIAXGLIREDQXGEXGAVVAGDXPG |
| | RGDAEVTIFDGTGVGLQDLAVAXAVVELAKHKGVAQEVEI |

Preferably, the method of preparing a primary amine compound of general formula (IB) comprises the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises a conserved amino acid sequence selected from the sequences as set forth in SEQ ID NO 611 to 621, wherein R¹ and R² have the meanings as defined herein.

More preferably, the method of preparing a primary amine compound of general formula (IB) comprises the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises the conserved amino acid sequence as set forth in SEQ ID NO 611, wherein R¹ and R² have the meanings as defined herein.

More preferably, the method of preparing a primary amine compound of general formula (IB) comprises the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises the conserved amino acid sequence as set forth in SEQ ID NO 612, wherein R¹ and R² have the meanings as defined herein.

More preferably, the method of preparing a primary amine compound of general formula (IB) comprises the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises the conserved amino acid sequence as set forth in SEQ ID NO 617, wherein R¹ and R² have the meanings as defined herein.

More preferably, the method of preparing a primary amine compound of general formula (IB) comprises the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises the conserved amino acid sequence as set forth in SEQ ID NO 618, wherein R¹ and R² have the meanings as defined herein.

More preferably, the method of preparing a primary amine compound of general formula (IB) comprises the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises the conserved amino acid sequence as set forth in SEQ ID NO 619, wherein R¹ and R² have the meanings as defined herein.

More preferably, the method of preparing a primary amine compound of general formula (IB) comprises the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises the conserved amino acid sequence as set forth in SEQ ID NO 620, wherein R¹ and R² have the meanings as defined herein.

Most preferably, the method of preparing a primary amine compound of general formula (IB) comprises the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises the conserved amino acid sequence as set forth in SEQ ID NO 621, wherein R¹ and R² have the meanings as defined herein.

In one embodiment of the invention, the polypeptide of step A1) of the inventive method comprises an amino acid sequence of preferably at least 80%, more preferably at least 85%, more preferably at least 87%, more preferably at least 90%, more preferably at least 92%, more preferably at least 95%, more preferably at least 96%, more preferably at least 97%, even more preferably at least 98%, even more preferably at least 99% and even more preferably 100% sequence identity to an amino acid sequence selected from SEQ ID Nos.: 300-598.

Thus, the present invention is directed to a method of preparing a primary amine compound of general formula (IB), comprising the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises a conserved amino acid as set forth in SEQ ID NO: 610; wherein R¹ and R² have the meanings as defined herein, and
   wherein the polypeptide comprises an amino acid sequence of at least 80% sequence identity to an amino acid sequence selected from SEQ ID Nos.: 300-598.

Moreover, the present invention is directed to a method of preparing a primary amine compound of general formula (IB), comprising the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises a conserved amino acid sequence as set forth in SEQ ID NO: 610; wherein R¹ and R² have the meanings as defined herein, and
   wherein the polypeptide consists of an amino acid sequence selected from SEQ ID Nos.: 300-598.

In another embodiment of the present invention, the polypeptide of step A1) of the inventive method comprises an amino acid sequence of preferably at least 80%, more preferably at least 85%, more preferably at least 87%, more preferably at least 90%, more preferably at least 92%, more preferably at least 95%, more preferably at least 96%, more preferably at least 97%, even more preferably at least 98%, even more preferably at least 99% and even more preferably 100% sequence identity to an amino acid sequence selected from SEQ ID Nos.: 300, 306, 321, 324, 325, 338, 346, 357, 374, 422, 434 and 459. Preferably, the polypeptide of step A1) of the inventive method consists of an amino acid sequence selected from SEQ ID Nos.: 300, 306, 321, 324, 325, 338, 346, 357, 374, 422, 434 and 459.

Thus, the present invention is directed to a method of preparing a primary amine compound of general formula (IB), comprising the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises a conserved amino acid sequence as set forth in SEQ ID NO: 610; wherein R¹ and R² have the meanings as defined herein, and
   wherein the polypeptide consists of an amino acid sequence selected from SEQ ID Nos.: 300, 306, 321, 324, 325, 338, 346, 357, 374, 422, 434 and 459.

The present invention is also directed to a method of preparing a primary amine compound of general formula (IB), comprising the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises a conserved amino acid sequence as set forth in SEQ ID NO: 610; wherein R¹ and R² have the meanings as defined herein, and
   wherein the polypeptide comprises an amino acid sequence of at least 80% sequence identity to an amino acid sequence selected from SEQ ID Nos.: 300, 306, 321, 324, 325, 338, 346, 357, 374, 422, 434 and 459.

In a further preferred embodiment of the present invention, the polypeptide of step A1) of the inventive method comprises an amino acid sequence of preferably at least 80%, more preferably at least 85%, more preferably at least 87%, more preferably at least 90%, more preferably at least 92%, more preferably at least 95%, more preferably at least 96%, more preferably at least 97%, even more preferably at least 98%, even more preferably at least 99% and even more preferably 100% sequence identity to an amino acid sequence of SEQ ID No.: 434. Preferably, the polypeptide of step A1) of the inventive method consists of an amino acid sequence of SEQ ID No.: 434.

Moreover, the present invention is directed to a method of preparing a primary amine compound of general formula (IB), comprising the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises a conserved amino acid sequence as set forth in SEQ ID NO: 610; wherein R¹ and R² have the meanings as defined herein; and
   wherein step B1) is performed at a substrate loading concentration of at least about 10 g/L, about 20 g/L, about 30 g/L, about 40 g/L, about 50 g/L, about 70 g/L, about 100 g/L, about 125 g/L, about 150 g/L, about 175 g/L or about 200 g/L or more with a percent conversion of at least about 40%, at least about 50%, at least about 60%, at least about 70%, at least about 80%, at least about 90%, at least about 95%, at least about 98%, or at least about 99%, in a reaction time of about 120 h or less, 72 h or less, about 48 h or less, about 36 h or less, or about 24 h less, under suitable reaction conditions.

In one embodiment, the method of preparing a primary amine compound of general formula (IB) further comprises a cofactor regeneration system capable of converting NADP⁺ to NADPH, or preferably NAD⁺ to NADH. Preferably, the cofactor regeneration systems comprises formate and formate dehydrogenase (FDH), glucose and glucose dehydrogenase (GDH), glucose-6-phosphate and glucose-6-phosphate dehydrogenase, a secondary alcohol and alcohol dehydrogenase or phosphate and phosphite dehydrogenase. Especially preferred is, when the cofactor regeneration systems comprises formate and formate dehydrogenase (FDH).

Thus, the present invention is directed to a method of preparing a primary amine compound of general formula (IB), comprising the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises a conserved amino acid sequence as set forth in SEQ ID NO: 610; wherein R¹ and R² have the meanings as defined herein, and
   wherein the cofactor is NADH.

Preferably, the method of preparing a primary amine compound of general formula (IB) comprises the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises a conserved amino acid sequence selected from the sequences as set forth in SEQ ID NO: 611 to 621, wherein R¹ and R² have the meanings as defined herein, and wherein the cofactor is NADH.

Preferably, the method of preparing a primary amine compound of general formula (IB) comprises the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises a conserved amino acid sequence as set forth in SEQ ID NO: 611, wherein R¹ and R² have the meanings as defined herein, and
   wherein the cofactor is NADH.

Preferably, the method of preparing a primary amine compound of general formula (IB) comprises the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises a conserved amino acid sequence as set forth in SEQ ID NO: 617, wherein R¹ and R² have the meanings as defined herein, and
   wherein the cofactor is NADH.

Preferably, the method of preparing a primary amine compound of general formula (IB) comprises the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises a conserved amino acid sequence as set forth in SEQ ID NO: 618, wherein R¹ and R² have the meanings as defined herein, and
   wherein the cofactor is NADH.

Preferably, the method of preparing a primary amine compound of general formula (IB) comprises the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises a conserved amino acid sequence as set forth in SEQ ID NO: 619, wherein R¹ and R² have the meanings as defined herein, and
   wherein the cofactor is NADH.

Preferably, the method of preparing a primary amine compound of general formula (IB) comprises the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises a conserved amino acid sequence as set forth in SEQ ID NO: 620, wherein R¹ and R² have the meanings as defined herein, and
   wherein the cofactor is NADH.

Preferably, the method of preparing a primary amine compound of general formula (IB) comprises the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises a conserved amino acid sequence as set forth in SEQ ID NO: 621, wherein R¹ and R² have the meanings as defined herein, and
   wherein the cofactor is NADH.

In case the primary imine compound of formula (IIA) is prochiral, a chiral amine of formula (IB) is formed (as indicated by the asterisk sign) by the inventive method. Thus, the polypeptides having the enzymatic activity of an imine reductase are capable of inducing stereoselectivity in the reduction of primary amines to imines as shown in FIG. 2C, thereby forming one stereocenter in a primary amine compound of formula (IB).

Thus, the present invention is also directed to a method of preparing a primary amine compound of general formula (IB), comprising the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises a conserved amino acid sequence as set forth in SEQ ID NO: 610; wherein R¹ and R² have the meanings as defined herein; and
   wherein the primary amine of general formula (IB) is formed as a single stereo-isomer.

It will be appreciated that the inventive method leads within its scope to all stereoisomers of the primary amine of general formula (IB). The term “(R) or (S) isomer” as used herein means that in formulae (I), (IA), (IB), (IC) and (V), the stereocenter which is marked with an asterisk, may be in (R) or (S) configuration. The (R) or (S) configuration is determined as follows. First, priorities are assigned to all substituents bonded to the carbon atom of the stereocenter according to the Cahn-Ingold-Prelog priority rules which have a well defined meaning in the art of organic chemistry. Second, the configuration of the stereocenter which is marked with an asterisk is determined based on the priority of the substituents bonded to the carbon atom of the stereocenter.

In some embodiments, (R)-configured primary amines are formed, provided that the amino group has the highest priority and R¹ is the higher priority substituent compared to R². Thus, the present invention is directed to a method of preparing a primary amine compound of general formula (R)-(IB)

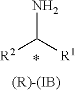

wherein R¹ and R² are independently selected from a hydrogen atom, and optionally substituted alkyl, alkenyl, alkynyl, alkoxy, carboxy, aminocarbonyl, thiocarbonyl, heteroalkyl, heteroalkenyl, heteroalkynyl, carboxyalkyl, aminoalkyl, haloalkyl, alkylthioalkyl, cycloalkyl, aryl, arylalkyl, heterocycloalkyl, heteroaryl, and heteroarylalkyl; and optionally R¹ and R² are linked to form a 3-membered to 10-membered ring, and wherein the amino group has the highest priority of all substituents and R¹ is the higher priority substituent compared to R²;
comprising:
A1) Providing an imine compound of general formula (IIA)

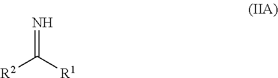

wherein R¹ and R² have the meanings as defined above; and
B1) Reacting imine compound of general formula (IIA) with a polypeptide having the enzymatic activity of imine reductase in the presence of a cofactor, to afford primary amine of general formula (R)-(IB),
   wherein the polypeptide comprises a conserved amino acid sequence as set forth in SEQ ID NO: 610.

Preferably, the polypeptide comprises a conserved amino acid sequence selected from the sequences as set forth in SEQ ID NO: 611 to 621. More preferably, the polypeptide comprises a conserved amino acid sequence as set forth in SEQ ID NO 611. More preferably, the polypeptide comprises a conserved amino acid sequence as set forth in SEQ ID NO 617. More preferably, the polypeptide comprises a conserved amino acid sequence as set forth in SEQ ID NO 618. More preferably, the polypeptide comprises a conserved amino acid sequence as set forth in SEQ ID NO 619. More preferably, the polypeptide comprises a conserved amino acid sequence as set forth in SEQ ID NO 620. More preferably, the polypeptide comprises a conserved amino acid sequence as set forth in SEQ ID NO 621.

In some embodiments, (S)-configured primary amines are formed, provided that the amino group has the highest priority and R² is the higher priority substituent compared to R¹. Thus, the present invention is directed to a method of preparing a primary amine compound of general formula (S)-(IB)

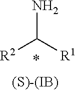

wherein R¹ and R² are independently selected from a hydrogen atom, and optionally substituted alkyl, alkenyl, alkynyl, alkoxy, carboxy, aminocarbonyl, thiocarbonyl, heteroalkyl, heteroalkenyl, heteroalkynyl, carboxyalkyl, aminoalkyl, haloalkyl, alkylthioalkyl, cycloalkyl, aryl, arylalkyl, heterocycloalkyl, heteroaryl, and heteroarylalkyl; and optionally R¹ and R² are linked to form a 3-membered to 10-membered ring, and wherein the amino group has the highest priority of all substituents and R² is the higher priority substituent compared to R¹;
comprising:
A1) Providing an imine compound of general formula (IIA)

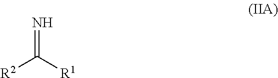

wherein R¹ and R² have the meanings as defined above; and
B1) Reacting imine compound of general formula (IIA) with a polypeptide having the enzymatic activity of an imine reductase in the presence of a cofactor, to afford primary amine of general formula (S)-(IB),
   wherein the polypeptide comprises a conserved amino acid sequence as set forth in SEQ ID NO: 610.

Further, the present invention is directed to a method of preparing a primary amine compound of general formula (IB), comprising the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises a conserved amino acid sequence as set forth in SEQ ID NO: 610; wherein R¹ and R² have the meanings as defined herein; and
   wherein the primary amine of general formula (IB) is a chiral amine formed in an enantiomeric excess or diastereomeric excess of greater than 90%, 91%, 92%, 93%, 94%, 95%, 96%, 97%, 98%, 99%, 99.5%, or greater.

The imine reductases used in the inventive method of preparing primary amines also catalyze the reduction of chiral or pro-chiral imines or even more precisely of chiral or pro-chiral primary imines to the corresponding chiral primary amines, as illustrated in FIG. 2C, but do not catalyze the reaction of a chiral or pro-chiral secondary imine to the corresponding chiral secondary amine and also not the reductive amination of a chiral or pro-chiral carbonyl compound and an amine substrate to the corresponding chiral tertiary amine as illustrated in FIG. 2A.

The term “chiral imine” or “chiral primary imine” refers to an imine of general formula (IIA), wherein the substituent R¹ and/or R² is/are chiral, i.e. comprise at least one stereogenic center so that primary amines are obtained having an additional stereogenic center at the carbon atom attached to the amine nitrogen atom as shown in general formula (IB).

The term “pro-chiral imine” or “pro-chiral primary imine” refers to an imine of general formula (IIA), wherein the substituents R¹ and R² are different from each other but not chiral, i.e. neither the substituent R¹ nor the substituent R² comprises a stereogenic center so that primary amines are obtained having exactly one stereogenic center at the carbon atom attached to the amine nitrogen atom as shown in general formula (IB).

In one embodiment of the present invention, the imine compound and even more precisely the primary imine compound of general formula (IIA) in step A1) is formed in-situ by reacting a ketone or an aldehyde with ammonia or an ammonium salt. Thus, the present invention is also directed to a method of preparing a primary amine compound of general formula (IB), comprising the steps of:
A2) Providing a carbonyl compound of general formula (III) and ammonia or an ammonium salt

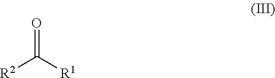

B2) Reacting the carbonyl compound of general formula (III) with ammonia or an ammonium salt in the presence of a polypeptide having the enzymatic activity of an imine reductase and a cofactor, to afford primary amine of general formula (IB),
   wherein the polypeptide comprises a conserved amino acid sequence of GXKXGX₈GXKXGGX₂PXNX₇NHQSX₃LFX₄GX₈NX₂TAXRTAAX₄SX₃LX₈GX₂GAGXQ X₃QX₁₅WNX₃₉SX₁₅HX₃MGTDTX₂KXEX₁₃DX₃QX₄GEXQX₁₆GX₉RX₆TX₂DGXGX₃QDX A (SEQ ID NO: 610), and wherein R¹ and R² have the meanings as defined herein. Preferably, the polypeptide comprises a conserved amino acid sequence selected from the sequences as set forth in SEQ ID NO: 611 to 621. More preferably, the polypeptide comprises a conserved amino acid sequence as set forth in SEQ ID NO 617. More preferably, the polypeptide comprises a conserved amino acid sequence as set forth in SEQ ID NO 618. More preferably, the polypeptide comprises a conserved amino acid sequence as set forth in SEQ ID NO 619. More preferably, the polypeptide comprises a conserved amino acid sequence as set forth in SEQ ID NO 620. More preferably, the polypeptide comprises a conserved amino acid sequence as set forth in SEQ ID NO 621.

Preferably, the ammonium salt is selected from ammonium chloride, ammonium bromide, ammonium formate or ammonium acetate.

Alternatively, the present invention is directed to a method of preparing a primary amine of general formula (IB) is formed by reacting a carbonyl compound with ammonia or an ammonium salt comprising the steps of:
A2) Providing a carbonyl compound of general formula (III)

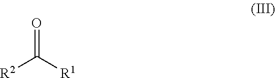

and ammonia or an ammonium salt in order to form the primary imine of general formula (IIA);
B2) Reacting the formed primary imine of general formula (IIA) with a polypeptide having the enzymatic activity of an imine reductase and a cofactor to afford the primary amine of general formula (IB),
   wherein the polypeptide comprises a conserved amino acid sequence as set forth in SEQ ID NO: 610.

Alternatively, the present invention is directed to a method of preparing a primary amine of general formula (IB) is formed in-situ by reacting a carbonyl compound with ammonia or an ammonium salt comprising the steps of:
A2) Providing a carbonyl compound of general formula (III)

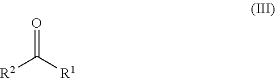

and ammonia or an ammonium salt in order to form in-situ the primary imine of general formula (IIA);
B2) Reacting the in-situ formed primary imine of general formula (IIA) with a polypeptide having the enzymatic activity of an imine reductase and a cofactor to afford the primary amine of general formula (IB),
   wherein the polypeptide comprises a conserved amino acid sequence as set forth in SEQ ID NO: 610.

Preferably, the polypeptide comprises a conserved amino acid sequence selected from the sequences as set forth in SEQ ID NO: 611 to 621. More preferably, the polypeptide comprises a conserved amino acid sequence as set forth in SEQ ID NO 617. More preferably, the polypeptide comprises a conserved amino acid sequence as set forth in SEQ ID NO 618. More preferably, the polypeptide comprises a conserved amino acid sequence as set forth in SEQ ID NO 619. More preferably, the polypeptide comprises a conserved amino acid sequence as set forth in SEQ ID NO 620. More preferably, the polypeptide comprises a conserved amino acid sequence as set forth in SEQ ID NO 621.

In one embodiment of the present invention, the polypeptide used in the inventive method is an engineered variant of the wild-type imine reductase polypeptide selected from SEQ ID NO: 300-598 that exhibits improved enzyme properties as compared to the naturally occurring imine reductase, including among others, imine reductase activity, substrate specificity, and selectivity and is still capable of catalyzing the reduction of an primary imine compound of formula (IIA) to a primary amine of formula (IB).

In one embodiment of the present invention the polypeptide used in the inventive method comprises the amino acid sequence selected from SEQ ID NO: 300-598, wherein the amino acid sequence can comprise deletions of one or more amino acids, 2 or more amino acids, 3 or more amino acids, 4 or more amino acids, 5 or more amino acids, 6 or more amino acids, 8 or more amino acids, 10 or more amino acids, 15 or more amino acids, or 20 or more amino acids, up to 10% of the total number of amino acids, up to 10% of the total number of amino acids, up to 20% of the total number of amino acids, or up to 30% of the total number of amino acids of the imine reductase polypeptides, where the associated functional activity and/or improved properties of the polypeptide having the enzymatic activity described herein is maintained. In some embodiments, the deletions can comprise 1-2, 1-3, 1-4, 1-5, 1-6, 1-7, 1-8, 1-9, 1-10, 1-15, 1-20, 1-21, 1-22, 1-23, 1-24, 1-25, 1-30, 1-35, 1-40, 1-45, or 1-50 amino acid residues. In some embodiments, the number of deletions can be 1, 2, 3, 4, 5, 6, 7, 8, 9, 10, 11, 12, 13, 14, 15, 16, 17, 18, 19, 20, 21, 22, 23, 24, 25, 30, 30, 35, 40, 45, or 50 amino acid residues. In some embodiments, the deletions can comprise deletions of 1, 2, 3, 4, 5, 6, 7, 8, 9, 10, 11, 12, 13, 14, 15, 16, 18, 20, 21, 22, 23, 24, or 25 amino acid residues.

In one embodiment of the present invention the polypeptide used in the inventive method comprises the amino acid sequence selected from SEQ ID NO: 300, 306, 321, 324, 325, 338, 346, 357, 374, 422, 434 and 459, wherein the amino acid sequence can comprise deletions of one or more amino acids, 2 or more amino acids, 3 or more amino acids, 4 or more amino acids, 5 or more amino acids, 6 or more amino acids, 8 or more amino acids, 10 or more amino acids, 15 or more amino acids, or 20 or more amino acids, up to 10% of the total number of amino acids, up to 10% of the total number of amino acids, up to 20% of the total number of amino acids, or up to 30% of the total number of amino acids of the imine reductase polypeptides, where the associated functional activity and/or improved properties of the polypeptide having the enzymatic activity described herein is maintained. In some embodiments, the deletions can comprise 1-2, 1-3, 1-4, 1-5, 1-6, 1-7, 1-8, 1-9, 1-10, 1-15, 1-20, 1-21, 1-22, 1-23, 1-24, 1-25, 1-30, 1-35, 1-40, 1-45, or 1-50 amino acid residues. In some embodiments, the number of deletions can be 1, 2, 3, 4, 5, 6, 7, 8, 9, 10, 11, 12, 13, 14, 15, 16, 17, 18, 19, 20, 21, 22, 23, 24, 25, 30, 30, 35, 40, 45, or 50 amino acid residues. In some embodiments, the deletions can comprise deletions of 1, 2, 3, 4, 5, 6, 7, 8, 9, 10, 11, 12, 13, 14, 15, 16, 18, 20, 21, 22, 23, 24, or 25 amino acid residues.

In one embodiment of the present invention the polypeptide used in the inventive method comprises the amino acid sequence of SEQ ID NO: 434, wherein the amino acid sequence can comprise deletions of one or more amino acids, 2 or more amino acids, 3 or more amino acids, 4 or more amino acids, 5 or more amino acids, 6 or more amino acids, 8 or more amino acids, 10 or more amino acids, 15 or more amino acids, or 20 or more amino acids, up to 10% of the total number of amino acids, up to 10% of the total number of amino acids, up to 20% of the total number of amino acids, or up to 30% of the total number of amino acids of the imine reductase polypeptides, where the associated functional activity and/or improved properties of the polypeptide having the enzymatic activity described herein is maintained. In some embodiments, the deletions can comprise 1-2, 1-3, 1-4, 1-5, 1-6, 1-7, 1-8, 1-9, 1-10, 1-15, 1-20, 1-21, 1-22, 1-23, 1-24, 1-25, 1-30, 1-35, 1-40, 1-45, or 1-50 amino acid residues. In some embodiments, the number of deletions can be 1, 2, 3, 4, 5, 6, 7, 8, 9, 10, 11, 12, 13, 14, 15, 16, 17, 18, 19, 20, 21, 22, 23, 24, 25, 30, 30, 35, 40, 45, or 50 amino acid residues. In some embodiments, the deletions can comprise deletions of 1, 2, 3, 4, 5, 6, 7, 8, 9, 10, 11, 12, 13, 14, 15, 16, 18, 20, 21, 22, 23, 24, or 25 amino acid residues.

In one embodiment of the present invention the polypeptide used in the inventive method comprises the amino acid sequence selected from SEQ ID NO: 300-598, wherein the amino acid sequence can comprise insertions of one or more amino acids, 2 or more amino acids, 3 or more amino acids, 4 or more amino acids, 5 or more amino acids, 6 or more amino acids, 8 or more amino acids, 10 or more amino acids, 15 or more amino acids, 20 or more amino acids, 30 or more amino acids, 40 or more amino acids, or 50 or more amino acids, where the associated functional activity and/or improved properties of the polypeptide having the enzymatic activity of an imine reductase described herein is maintained. The insertions can be to amino or carboxy terminus, or internal portions of the imine reductase polypeptide.

In one embodiment of the present invention the polypeptide used in the inventive method comprises the amino acid sequence selected from SEQ ID NO: 300-598, wherein the amino acid sequence has optionally 1-2, 1-3, 1-4, 1-5, 1-6, 1-7, 1-8, 1-9, 1-10, 1-15, 1-20, 1-21, 1-22, 1-23, 1-24, 1-25, 1-30, 1-35, 1-40, 1-45, or 1-50 amino acid residue deletions, insertions and/or substitutions. In some embodiments, the number of amino acid sequence has optionally 1, 2, 3, 4, 5, 6, 7, 8, 9, 10, 11, 12, 13, 14, 15, 16, 17, 18, 19, 20, 21, 22, 23, 24, 25, 30, 30, 35, 40, 45, or 50 amino acid residue deletions, insertions and/or substitutions. In some embodiments, the amino acid sequence has optionally 1, 2, 3, 4, 5, 6, 7, 8, 9, 10, 11, 12, 13, 14, 15, 16, 18, 20, 21, 22, 23, 24, or 25 amino acid residue deletions, insertions and/or substitutions.

In some embodiments, the polypeptides used in the inventive method can be in the form of fusion polypeptides in which the engineered polypeptides are fused to other polypeptides, such as, by way of example and not limitation, antibody tags (e.g., myc epitope), purification sequences (e.g., His tags for binding to metals), and cell localization signals (e.g., secretion signals). Thus, the engineered polypeptides described herein can be used in the inventive method with or without fusions to other polypeptides.

It is to be understood that the polypeptides described herein are not restricted to the genetically encoded amino acids. In addition to the genetically encoded amino acids, the polypeptides described herein may be comprised, either in whole or in part, of naturally-occurring and/or synthetic non-encoded amino acids. Therefore, the polypeptides used in the inventive method may comprise non-encoded amino acids selected from the group comprising or consisting of: the D-stereomers of the genetically-encoded amino acids, 2,3-diaminopropionic acid (Dpr), α-aminoisobutyric acid (Aib), ε-aminohexanoic acid (Aha), δ-aminovaleric acid (Ava), N-methylglycine or sarcosine (MeGly or Sar), ornithine (Orn), citrulline (Cit), t-butylalanine (Bua), t-butylglycine (Bug), N-methylisoleucine (Melle), phenylglycine (Phg), cyclohexyl-alanine (Cha), norleucine (Nle), naphthylalanine (Nal), 2-chlorophenylalanine (Ocf), 3-chlorophenylalanine (Mcf), 4-chlorophenylalanine (Pcf), 2-fluorophenylalanine (Off), 3-fluorophenylalanine (Mff), 4-fluorophenylalanine (Pff), 2-bromophenylalanine (Obf), 3-bromophenylalanine (Mbf), 4-bromophenylalanine (Pbf), 2-methylphenylalanine (Omf), 3-methylphenylalanine (Mmf), 4-methylphenylalanine (Pmf), 2-nitrophenyl-alanine (Onf), 3-nitrophenylalanine (Mnf), 4-nitrophenylalanine (Pnf), 2-cyanophenyl-alanine (Ocf), 3-cyanophenylalanine (Mcf), 4-cyanophenylalanine (Pcf), 2-trifluoro-methylphenylalanine (Otf), 3-trifluoromethylphenylalanine (Mtf), 4-trifluoromethyl-phenylalanine (Ptf), 4-aminophenylalanine (Paf), 4-iodophenylalanine (Pif), 4-amino-methylphenylalanine (Pamf), 2,4-dichlorophenylalanine (Opef), 3,4-dichloro-phenylalanine (Mpcf), 2,4-difluorophenylalanine (Opff), 3,4-difluorophenylalanine (Mpff), pyrid-2-ylalanine (2pAla), pyrid-3-ylalanine (3pAla), pyrid-4-ylalanine (4pAla), naphth-1-ylalanine (1nAla), naphth-2-ylalanine (2nAla), thiazolylalanine (taAla), benzothienylalanine (bAla), thienylalanine (tAla), furylalanine (fAla), homophenylalanine (hPhe), homotyrosine (hTyr), homotryptophan (hTrp), penta-fluorophenylalanine (5ff), styrylkalanine (sAla), authrylalanine (aAla), 3,3-diphenyl-alanine (Dfa), 3-amino-5-phenypentanoic acid (Afp), penicillamine (Pen), 1,2,3,4-tetrahydroisoquinoline-3-carboxylic acid (Tic), β-2-thienylalanine (Thi), methionine sulfoxide (Mso), N(w)-nitroarginine (nArg), homolysine (hLys), phosphonomethylphenylalanine (pmPhe), phosphoserine (pSer), phosphothreonine (pThr), homoaspartic acid (hAsp), homoglutanic acid (hGlu), 1-aminocyclopent-(2)-ene-4 carboxylic acid, 1-aminocyclopent-(3)-ene-4 carboxylic acid, pipecolic acid (PA), azetidine-3-carboxylic acid (ACA), 1-aminocyclopentane-3-carboxylic acid, allylglycine (aOly), propargylglycine (pgGly), homoalanine (hAla), norvaline (nVal), homoleucine (hLeu), homovaline (hVal), homoisoleucine (hIle), homoarginine (hArg), N-acetyl lysine (AcLys), 2,4-diaminobutyric acid (Dbu), 2,3-diaminobutyric acid (Dab), N-methylvaline (MeVal), homocysteine (hCys), homoserine (hSer), hydroxyproline (Hyp) and homoproline (hPro). Those of skill in the art will recognize that amino acids or residues bearing side chain protecting groups may also comprise the polypeptides used in the inventive method. Non-limiting examples of such protected amino acids, which in this case belong to the aromatic category, include (protecting groups listed in parentheses), but are not limited to: Arg(tos), Cys(methylbenzyl), Cys (nitropyridinesulfenyl), Glu(b-benzylester), Gln(xanthyl), Asn(N-b-xanthyl), His(bom), His(benzyl), His(tos), Lys(fmoc), Lys(tos), Ser(O-benzyl), Thr (O-benzyl) and Tyr(O-benzyl). Non-encoding amino acids that are conformationally constrained of which the polypeptides described herein may be composed include, but are not limited to, N-methyl amino acids (D-configuration), 1-aminocyclopent-(2)-ene-4-carboxylic acid, 1-aminocyclopent-(3)-ene-4-carboxylic acid, pipecolic acid, azetidine-3-carboxylic acid, homoproline (hPro) and 1-aminocyclopentane-3-carboxylic acid.

In some embodiments, the polypeptides having the enzymatic activity of an imine reductase are used in the inventive method in various forms, for example, such as an isolated preparation, as a substantially purified enzyme, whole cells transformed with gene(s) encoding the enzyme, and/or as cell extracts and/or lysates of such cells. The enzymes can be lyophilized, spraydried, precipitated or be in the form of a crude paste.

In some embodiments, the polypeptides used in the inventive method are provided on a solid support, such as a membrane, resin, solid carrier, or other solid phase material. A solid support can be composed of organic polymers such as polystyrene, polyethylene, polypropylene, polyfluoroethylene, polyethyleneoxy, and polyacrylamide, as well as co-polymers and grafts thereof. A solid support can also be inorganic, such as glass, silica, controlled pore glass (CPG), reverse phase silica or metal, such as gold or platinum. The configuration of a solid support can be in the form of beads, spheres, particles, granules, a gel, a membrane or a surface. Surfaces can be planar, substantially planar, or non-planar. Solid supports can be porous or non-porous, and can have swelling or non-swelling characteristics. A solid support can be configured in the form of a well, depression, or other container, vessel, feature, or location.

In some embodiments, the polypeptides used in the inventive method are immobilized on a solid support such that they retain their activity, stereoselectivity, and/or other properties. In such embodiments, the immobilized polypeptides can facilitate the reduction of an imine compound of formula (IIA) to a primary amine compound of formula (IB), and after the reaction is complete the immobilized polypeptides are easily retained (e.g., by retaining beads on which polypeptide is immobilized) and then reused or recycled in subsequent reactions. Such immobilized biocatalytic processes allow for further efficiency and cost reduction.

Accordingly, the present invention is further directed to a method of preparing a primary amine compound of general formula (IB), comprising the steps of:
A1) Providing an imine compound of general formula (IIA); and
B1) Reacting imine compound of general formula (IIA) with a polypeptide having the enzymatic activity of an imine reductase in the presence of a cofactor, to afford primary amine of general formula (IB),
   wherein the polypeptide comprises the conserved amino acid sequence of GXKXGX₈GXKXGGX₂PXNX₇NHQSX₃LFX₄GX₈NX₂TAXRTAAX₄SX₃LX₈GX₂GAGXQ X₃QX₁₅WNX₃₉SX₁₅HX₃MGTDTX₂KXEX₁₃DX₃QX₄GEXQX₁₆GX₉RX₆TX₂DGXGX₃QDX A (SEQ ID NO: 610), wherein R¹ and R² have the meanings as defined herein; and wherein the polypeptide is bound or immobilized on a solid support.

Methods of enzyme immobilization are well-known in the art. The polypeptides can be bound non-covalently or covalently. Various methods for conjugation and immobilization of enzymes to solid supports (e.g., resins, membranes, beads, glass, etc.) are well known in the art and described in e.g.,: Yi et al., Process Biochemistry 2007, 42, 895; Martin et al., Applied Microbiology and Biotechnology 2007, 76, 843; Koszelewski et al., Journal of Molecular Catalysis B: Enzymatic, 2010, 63, 39; Truppo et al., Org. Process Res. Dev., 2011, 15, 1033; Hermanson, G. T., Bioconjugate Techniques, Second Edition, Academic Press (2008); Mateo et al., Biotechnology Progress, 2002, 18, 629; and Bioconjugation Protocols: Strategies and Methods, In Methods in Molecular Biology, C. M. Niemeyer ed., Humana Press (2004). Solid supports useful for immobilizing the polypeptides of the present invention include but are not limited to beads or resins comprising polymethacrylate with epoxide functional groups, polymethacrylate with amino epoxide functional groups, styrene/DVB copolymer or polymethacrylate with octadecyl functional groups. Exemplary solid supports useful for immobilizing the engineered imine reductase polypeptides of the present disclosure include, but are not limited to, chitosan beads, Eupergit C, and SEPABEADs (Mitsubishi), including the following different types of SEPABEAD: EC-EP, EC-HFA/S, EXA252, EXE119 and EXE120.

### Kits

The imine reductase polypeptides used in the inventive method of preparing primary amines can be provided in the form of kits. The enzymes in the kits may be present individually or as a plurality of enzymes. The kits can further include reagents for carrying out the enzymatic reactions, substrates for assessing the activity of enzymes, as well as reagents for detecting the products. The kits can also include reagent dispensers and instructions for use of the kits.

The kits described herein can include arrays comprising a plurality of different polypeptides having the enzymatic activity of an imine reductase at different addressable positions, wherein the different polypeptides are different variants of a reference sequence each having at least one different improved enzyme property. In some embodiments, a plurality of polypeptides immobilized on solid supports can be configured on an array at various locations, addressable for robotic delivery of reagents, or by detection methods and/or instruments. The array can be used to test a variety of substrate compounds for conversion by the polypeptides.

In one embodiment the kit for preparing a primary amine compound of general formula (IB) from amino alcohol compound of general formula (V)

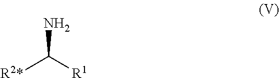

wherein R¹ represents —COOH, R²* represents —C(OH)Z¹Z²
wherein Z¹ represents —H or —CH₃, and
Z² represents —H, —CH₃, —CH₂CH₃, —(CH₂)₂CH₃, —COOH, —CONH₂, —CH₂OH, —CH₂SH, —CH₂COOH or —CH₂CONH₂,
comprises:
a) a polypeptide comprising an amino acid sequence of at least 80% sequence identity to an amino acid sequence selected from SEQ ID NO: 300-598 which has the enzymatic activity of an imine reductase, and
b) a polypeptide comprising an amino acid sequence of SEQ ID NO: 602 which has the enzymatic activity of a β-hydroxyaspartate dehydratase.

In one embodiment the kit for preparing a primary amine compound of general formula (IB) comprises
a) a polypeptide comprising an amino acid sequence of at least 80% sequence identity to an amino acid sequence selected from SEQ ID NO: 300-598, which has the enzymatic activity of an imine reductase, and
b) a polypeptide comprising an amino acid sequence of at least 80% sequence identity to SEQ ID NO: 602 which has the enzymatic activity of a β-hydroxy-aspartate dehydratase.

In one embodiment the kit for preparing a primary amine compound of general formula (IB) comprises
a) a polypeptide comprising an amino acid sequence of at least 80% sequence identity to an amino acid sequence selected from SEQ ID NO: 300, 306, 321, 324, 325, 338, 346, 357, 374, 422, 434 and 459, which has the enzymatic activity of an imine reductase, and
b) a polypeptide comprising an amino acid sequence of at least 80% sequence identity to SEQ ID NO: 602 which has the enzymatic activity of a β-hydroxy-aspartate dehydratase.

In one embodiment the kit for preparing a primary amine compound of general formula (IB) comprises
a) a polypeptide comprising an amino acid sequence of at least 80% sequence identity to SEQ ID NO: 434, which has the enzymatic activity of an imine reductase, and
b) a polypeptide comprising an amino acid sequence of at least 80% sequence identity to SEQ ID NO: 602 which has the enzymatic activity of a β-hydroxy-aspartate dehydratase.

In one embodiment the kit for preparing a primary amine compound of general formula (IB) further comprises the cofactor NADH. In another embodiment, the kit comprises the cofactor NADPH. Thus, the present invention is also directed to a kit for preparing a primary amine compound of general formula (IB) from amino alcohol compound of general formula (V) comprising:
a) a polypeptide comprising an amino acid sequence of at least 80% sequence identity to an amino acid sequence selected from SEQ ID NO: 300-598, which has the enzymatic activity of an imine reductase,
b) a polypeptide comprising an amino acid sequence of SEQ ID NO: 602 which has the enzymatic activity of a β-hydroxyaspartate dehydratase, and
c) a cofactor selected from NADH or NADPH.

In one embodiment the kit for preparing a primary amine compound of general formula (IB) comprises
a) a polypeptide comprising an amino acid sequence of at least 80% sequence identity to an amino acid sequence selected from SEQ ID NO: 300-598, which has the enzymatic activity of an imine reductase,
b) a polypeptide comprising an amino acid sequence of at least 80% sequence identity to SEQ ID NO: 602 which has the enzymatic activity of a β-hydroxy-aspartate dehydratase, and
c) a cofactor selected from NADH or NADPH.

Preferably, the kit comprises
a) a polypeptide comprising an amino acid sequence of at least 80% sequence identity to an amino acid sequence selected from SEQ ID NO: 300-598, which has the enzymatic activity of an imine reductase,
b) a polypeptide comprising an amino acid sequence of SEQ ID NO: 6 which has the enzymatic activity of a β-hydroxyaspartate dehydratase, and
c) the cofactor NADH.

Preferably, the kit comprises
a) a polypeptide comprising an amino acid sequence of at least 80% sequence identity to an amino acid sequence selected from SEQ ID NO: 300-598, which has the enzymatic activity of an imine reductase,
b) a polypeptide comprising an amino acid sequence of at least 80% sequence identity to SEQ ID NO: 602 which has the enzymatic activity of a β-hydroxy-aspartate dehydratase, and
c) the cofactor NADH.

In another embodiment, the kit comprises a cofactor regeneration system capable of converting NADP⁺ to NADPH or, preferably NAD⁺ to NADH.

Thus, the herein disclosed kit for preparing a primary amine compound of general formula (IB) from amino alcohol compound of general formula (V) comprises:
a) a polypeptide comprising an amino acid sequence of at least 80% sequence identity to an amino acid sequence selected from SEQ ID NO: 300-598, which has the enzymatic activity of an imine reductase,
b) a polypeptide comprising an amino acid sequence of SEQ ID NO: 602 which has the enzymatic activity of a β-hydroxyaspartate dehydratase, and
c) a cofactor generation system for the conversion of NAD⁺ to NADH and/or NADP⁺ to NADPH.

Alternatively, the kit for preparing an amine compound of general formula (I) comprises
a) a polypeptide comprising an amino acid sequence of at least 80% sequence identity to an amino acid sequence selected from SEQ ID NO: 300-598, which has the enzymatic activity of an imine reductase,
b) a polypeptide comprising an amino acid sequence of at least 80% sequence identity to SEQ ID NO: 602 which has the enzymatic activity of a β-hydroxy-aspartate dehydratase, and
c) a cofactor generation system for the conversion of NAD⁺ to NADH and/or NADP⁺ to NADPH.

Preferably, the kit comprises
a) a polypeptide comprising an amino acid sequence of at least 80% sequence identity to an amino acid sequence selected from SEQ ID NO: 300-598, which has the enzymatic activity of an imine reductase,
b) a polypeptide comprising an amino acid sequence of SEQ ID NO: 602 which has the enzymatic activity of a β-hydroxyaspartate dehydratase, and
c) a cofactor generation system for the conversion of NAD⁺ to NADH, wherein the cofactor regeneration systems comprises formate and formate dehydrogenase.

Preferably, the kit comprises
a) a polypeptide comprising an amino acid sequence of at least 80% sequence identity to an amino acid sequence selected from SEQ ID NO: 300-598, which has the enzymatic activity of an imine reductase,
b) a polypeptide comprising an amino acid sequence of at least 80% sequence identity to SEQ ID NO: 602 which has the enzymatic activity of a β-hydroxy-aspartate dehydratase, and
c) a cofactor generation system for the conversion of NAD⁺ to NADH, wherein the cofactor regeneration systems comprises formate and formate dehydrogenase.

### Nucleic Acids Encoding Polypeptides Having the Enzymatic Activity of an Imine Reductase, Expression Vectors and Host Cells

The polypeptides having the enzymatic activity of an imine reductase disclosed herein can be encoded by nucleic acids, which may be operatively linked to one or more heterologous regulatory sequences that control gene expression to create a recombinant polynucleotide capable of expressing the polypeptide. Expression constructs containing heterologous nucleic acids encoding the polypeptides having the enzymatic activity of an imine reductase can be introduced into appropriate host cells to express the corresponding imine reductase polypeptide that is used in the inventive method.

As will be apparent to the skilled artisan, availability of a protein sequence and the knowledge of the codons corresponding to the various amino acids provide a description of all the nucleic acids capable of encoding the subject polypeptides. The degeneracy of the genetic code, where the same amino acids are encoded by alternative or synonymous codons, allows an extremely large number of nucleic acids to be made, all of which encode the imine reductase polypeptides. Thus, having knowledge of a particular amino acid sequence, those skilled in the art could make any number of different nucleic acids by simply modifying the sequence of one or more codons in a way which does not change the amino acid sequence of the protein. In this regard, the present disclosure specifically contemplates each and every possible variation of nucleic acids that could be made encoding the polypeptides described herein by selecting combinations based on the possible codon choices, and all such variations are to be considered specifically disclosed for any polypeptide having the enzymatic activity of an imine reductase described herein.

The codons are preferably selected to fit the host cell in which the protein is being produced. For example, preferred codons used in bacteria are used to express the gene in bacteria; preferred codons used in yeast are used for expression in yeast; and preferred codons used in mammals are used for expression in mammalian cells. In some embodiments, all codons need not be replaced to optimize the codon usage of the imine reductases since the natural sequence will comprise preferred codons and because use of preferred codons may not be required for all amino acid residues. Consequently, codon optimized nucleic acids encoding the polypeptides having the enzymatic activity of an imine reductase may contain preferred codons at about 40%, 50%, 60%, 70%, 80%, or greater than 90% of codon positions of the full length coding region.

Preferably, the nucleic acid comprises a nucleotide sequence encoding a polypeptide having the enzymatic activity of an imine reductase comprising the conserved amino as set forth in SEQ ID NO: 610. Preferably, the polypeptide comprises a conserved amino acid sequence selected from the sequences as set forth in SEQ ID NO: 611 to 621. More preferably, the polypeptide comprises a conserved amino acid sequence as set forth in SEQ ID NO 611. More preferably, the polypeptide comprises a conserved amino acid sequence as set forth in SEQ ID NO 617. More preferably, the polypeptide comprises a conserved amino acid sequence as set forth in SEQ ID NO 618. More preferably, the polypeptide comprises a conserved amino acid sequence as set forth in SEQ ID NO 619. More preferably, the polypeptide comprises a conserved amino acid sequence as set forth in SEQ ID NO 620. More preferably, the polypeptide comprises a conserved amino acid sequence as set forth in SEQ ID NO 621.

Preferably, the nucleic acid comprises a nucleotide sequence encoding the naturally occurring imine reductase polypeptide of SEQ ID NO: 300-598. The nucleic acid encoding a polypeptide having the enzymatic activity of an imine reductase, that is used in the inventive method, comprises a polynucleotide sequence having at least 80%, more preferably 85%, even more preferably 86%, even more preferably 87%, even more preferably 88%, even more preferably 89%, even more preferably 90%, even more preferably 91, even more preferably 92%, even more preferably, even more preferably 93%, even more preferably 94, even more preferably 95%, even more preferably 96%, even more preferably 97%, even more preferably 98% and most preferably 99% sequence identity to the nucleic acid sequence of SEQ ID NO: 300-598.

Preferably, the nucleic acid comprises a nucleotide sequence encoding the naturally occurring imine reductase polypeptide of SEQ ID NO: 300, 306, 321, 324, 325, 338, 346, 357, 374, 422, 434 and 459. The nucleic acid encoding a polypeptide having the enzymatic activity of an imine reductase, that is used in the inventive method, comprises a polynucleotide sequence having at least 80%, more preferably 85%, even more preferably 86%, even more preferably 87%, even more preferably 88%, even more preferably 89%, even more preferably 90%, even more preferably 91, even more preferably 92%, even more preferably, even more preferably 93%, even more preferably 94, even more preferably 95%, even more preferably 96%, even more preferably 97%, even more preferably 98% and most preferably 99% sequence identity to the nucleic acid sequence of SEQ ID NO: 300, 306, 321, 324, 325, 338, 346, 357, 374, 422, 434 and 459.

Preferably, the nucleic acid comprises a nucleotide sequence encoding the naturally occurring imine reductase polypeptide of SEQ ID NO: 434. In some embodiments, the nucleic acid encoding a polypeptide having the enzymatic activity of an imine reductase, that is used in the inventive method, comprises a polynucleotide sequence having at least 80%, more preferably 85%, even more preferably 86%, even more preferably 87%, even more preferably 88%, even more preferably 89%, even more preferably 90%, even more preferably 91, even more preferably 92%, even more preferably, even more preferably 93%, even more preferably 94, even more preferably 95%, even more preferably 96%, even more preferably 97%, even more preferably 98% and most preferably 99% sequence identity to the nucleic acid sequence of SEQ ID NO: 434.

Alternatively, the nucleic acid consists of a nucleotide sequence encoding the naturally occurring imine reductase polypeptide of SEQ ID NO: 300-598.

The nucleic acid as described herein is capable of hybridizing under highly stringent conditions to a polynucleotide sequence selected from SEQ ID NO: 1-299, or a complement thereof, and encodes a polypeptide having the enzymatic activity of an imine reductase.

The nucleic acid described herein further encodes a polypeptide having an imine reductase activity capable of converting a carbonyl compound of formula (III) and amine substrate compound of formula (IV), to an amine product compound of formula (I) and/or an imine compound of formula (II) or (IIA) to amine compound of formula (IA) or (IB), wherein the polypeptide comprises an amino acid sequence having at least 80%, more preferably 85%, more preferably 86%, more preferably 87%, more preferably 88%, more preferably 89%, more preferably 90%, more preferably 91%, more preferably 92%, more preferably 93%, more preferably 94%, more preferably 95%, more preferably 96%, more preferably 97%, more preferably 98%, more preferably 99% or more sequence identity to the amino acid sequence of SEQ ID NO: 300-598.

An isolated nucleic acid encoding a polypeptide may be manipulated in a variety of ways to provide for expression of the polypeptide having the enzymatic activity of an imine reductase. The nucleic acid encoding the polypeptides may be provided as expression vector where one or more control sequences are present to regulate the expression of the nucleic acids and/or polypeptides. Manipulation of the isolated polynucleotide prior to its insertion into a vector may be desirable or necessary depending on the expression vector. The techniques for modifying nucleic acids and polynucleotide sequences utilizing recombinant DNA methods are well known in the art.

The control sequences may include among others, promoters, leader sequence, polyadenylation sequence, propeptide sequence, signal peptide sequence, and transcription terminator. Suitable promoters can be selected based on the host cells used. For bacterial host cells, suitable promoters for directing transcription of the nucleic acid constructs of the present disclosure, include the promoters obtained from the E. coli lac operon, Streptomyces coelicolor agarase gene (dagA), Bacillus subtilis levansucrase gene (sacB), Bacillus licheniformis α-amylase gene (amyL), Bacillus stearothermophilus maltogenic amylase gene (amyM), Bacillus amyloliquefaciens α-amylase gene (amyQ), Bacillus licheniformis penicillinase gene (penP), Bacillus subtilis xylA and xylB genes and prokaryotic β-lactamase gene (Villa-Kamaroff et al., Proc. Natl Acad. Sci. 1978, 75, 3727), as well as the tac promoter (DeBoer et al., Proc. Natl Acad. Sci. 1983, 80, 21). Exemplary promoters for filamentous fungal host cells, include promoters obtained from the genes for Aspergillus oryzae TAKA amylase, Rhizomucor miehei aspartic proteinase, Aspergillus niger neutral α-amylase, Aspergillus niger acid stable α-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase and Fusarium oxysporum trypsin-like protease (WO1996/00787), as well as the NA2-tpi promoter (a hybrid of the promoters from the genes for Aspergillus niger neutral α-amylase and Aspergillus oryzae triose phosphate isomerase) and mutant, truncated and hybrid promoters thereof. Exemplary yeast cell promoters can be from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase. Other useful promoters for yeast host cells are described by Romanos et al., Yeast 1992, 8, 423.

The control sequence may also be a suitable transcription terminator sequence, a sequence recognized by a host cell to terminate transcription. The terminator sequence is operably linked to the 3′ terminus of the nucleic acid sequence encoding the polypeptide. Any terminator which is functional in the host cell of choice may be used in the present invention. For example, exemplary transcription terminators for filamentous fungal host cells can be obtained from the genes for Aspergillus oryzae TAKA amylase, Aspergillus niger glucoamylase, Aspergillus nidulans anthranilate synthase, Aspergillus niger α-glucosidase and Fusarium oxysporum trypsin-like protease. Exemplary terminators for yeast host cells can be obtained from the genes for Saccharomyces cerevisiae enolase, Saccharomyces cerevisiae cytochrome C (CYCI) and Saccharomyces cerevisiae glyceraldehyde-3-phosphate dehydrogenase. Other useful terminators for yeast host cells are described by Romanos et al., Yeast 1992, 8, 423.

The control sequence may also be a suitable leader sequence, a nontranslated region of an mRNA that is important for translation by the host cell. The leader sequence is operably linked to the 5′ terminus of the nucleic acid sequence encoding the polypeptide. Any leader sequence that is functional in the host cell of choice may be used. Exemplary leaders for filamentous fungal host cells are obtained from the genes for Aspergillus oryzae TAKA amylase and Aspergillus nidulans triose phosphate isomerase. Suitable leaders for yeast host cells are obtained from the genes for Saccharomyces cerevisiae enolase (ENO-1), Saccharomyces cerevisiae 3-phosphoglycerate kinase, Saccharomyces cerevisiae α-factor and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′ terminus of the nucleic acid sequence and which, when transcribed, is recognized by the host cell as a signal to add polyadenosine residues to transcribed mRNA. Any polyadenylation sequence which is functional in the host cell of choice may be used in the present invention. Exemplary polyadenylation sequences for filamentous fungal host cells can be from the genes for Aspergillus oryzae TAKA amylase, Aspergillus niger glucoamylase, Aspergillus nidulans anthranilate synthase, Fusarium oxysporum trypsin-like protease and Aspergillus niger α-glucosidase. Useful polyadenylation sequences for yeast host cells are described by Guo and Sherman, Mol Cell Bio 1995, 15, 5983.

The control sequence may also be a signal peptide coding region that codes for an amino acid sequence linked to the amino terminus of a polypeptide and directs the encoded polypeptide into the cell's secretory pathway. The 5′ end of the coding sequence of the nucleic acid sequence may inherently contain a signal peptide coding region naturally linked in translation reading frame with the segment of the coding region that encodes the secreted polypeptide. Alternatively, the 5′ end of the coding sequence may contain a signal peptide coding region that is foreign to the coding sequence. Any signal peptide coding region which directs the expressed polypeptide into the secretory pathway of a host cell of choice may be used in the present invention. Effective signal peptide coding regions for bacterial host cells are the signal peptide coding regions obtained from the genes for Bacillus NCIB 11837 maltogenic amylase, Bacillus stearothermophilus α-amylase, Bacillus licheniformis subtilisin, Bacillus licheniformis β-lactamase, Bacillus stearothermophilus neutral proteases (nprT, nprS, nprM), and Bacillus subtilis prsA. Further signal peptides are described by Simonen and Palva, Microbiol Rev 1993, 57, 109. Effective signal peptide coding regions for filamentous fungal host cells can be the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase and Humicola lanuginosa lipase. Useful signal peptides for yeast host cells can be from the genes for Saccharomyces cerevisiae α-factor and Saccharomyces cerevisiae invertase. Other useful signal peptide coding regions are described by Romanos et al., Yeast 1992, 8, 423.

The control sequence may also be a propeptide coding region that codes for an amino acid sequence positioned at the amino terminus of a polypeptide. The resultant polypeptide is known as a proenzyme or propolypeptide (or a zymogen in some cases). A propolypeptide can be converted to a mature active polypeptide by catalytic or autocatalytic cleavage of the propeptide from the propolypeptide. The propeptide coding region may be obtained from the genes for Bacillus subtilis alkaline protease (aprE), Bacillus subtilis neutral protease (nprT), Saccharomyces cerevisiae α-factor, Rhizomucor miehei aspartic proteinase and Myceliophthora thermophila lactase (WO1995/33836). Where both signal peptide and propeptide regions are present at the amino terminus of a polypeptide, the propeptide region is positioned next to the amino terminus of a polypeptide and the signal peptide region is positioned next to the amino terminus of the propeptide region.

It may also be desirable to add regulatory sequences, which allow the regulation of the expression of the polypeptide relative to the growth of the host cell. Examples of regulatory systems are those which cause the expression of the gene to be turned on or off in response to a chemical or physical stimulus, including the presence of a regulatory compound. In prokaryotic host cells, suitable regulatory sequences include the lac, tac, and trp operator systems. In yeast host cells, suitable regulatory systems include, as examples, the ADH2 system or GAL1 system. In filamentous fungi, suitable regulatory sequences include the TAKA α-amylase promoter, Aspergillus niger glucoamylase promoter and Aspergillus oryzae glucoamylase promoter.

Other examples of regulatory sequences are those which allow for gene amplification. In eukaryotic systems, these include the dihydrofolate reductase gene, which is amplified in the presence of methotrexate, and the metallothionein genes, which are amplified with heavy metals. In these cases, the nucleic acid sequence encoding the polypeptide of the present invention would be operably linked with the regulatory sequence.

Thus, herein disclosed is also a recombinant expression vector comprising a nucleic acid encoding a polypeptide having the enzymatic activity of an imine reductase, and one or more expression regulating regions such as a promoter and a terminator, a replication origin, etc., depending on the type of hosts into which they are to be introduced. The various nucleic acid and control sequences described above may be joined together to produce a recombinant expression vector which may include one or more convenient restriction sites to allow for insertion or substitution of the nucleic acid sequence encoding the polypeptide at such sites. Alternatively, the nucleic acid sequence of the present disclosure may be expressed by inserting the nucleic acid sequence or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g. a plasmid or virus), which can be conveniently subjected to recombinant DNA procedures and can bring about the expression of the polynucleotide sequence. The choice of the vector will typically depend on the compatibility of the vector with the host cell into which the vector is to be introduced. The vectors may be linear or closed circular plasmids.

The expression vector may be an autonomously replicating vector, i.e. a vector that exists as an extrachromosomal entity, the replication of which is independent of chromosomal replication, such as a plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

The expression vector may comprise a nucleic acid comprising a nucleotide sequence encoding a polypeptide comprising a conserved amino acid sequence as set forth in SEQ ID NO: 610. Preferably, the nucleotide sequence encodes a polypeptide comprising a conserved amino acid sequence selected from the sequences as set forth in SEQ ID NO: 611 to 621. More preferably, the nucleotide sequence encodes a polypeptide comprising a conserved amino acid sequence as set forth in SEQ ID NO 611. More preferably, the nucleotide sequence encodes a polypeptide comprising a conserved amino acid sequence as set forth in SEQ ID NO 617. More preferably, the nucleotide sequence encodes a polypeptide comprising a conserved amino acid sequence as set forth in SEQ ID NO 618. More preferably, the nucleotide sequence encodes a polypeptide comprising a conserved amino acid sequence as set forth in SEQ ID NO 619. More preferably, the nucleotide sequence encodes a polypeptide comprising a conserved amino acid sequence as set forth in SEQ ID NO 620. More preferably, the nucleotide sequence encodes a polypeptide comprising a conserved amino acid sequence as set forth in SEQ ID NO 621.

Preferably, the expression vector may comprise a nucleic acid comprising a nucleotide sequence encoding the naturally occurring imine reductase polypeptide selected from SEQ ID NO: 300-598. In some embodiments, the expression vector comprises a nucleic acid encoding a polypeptide having the enzymatic activity of an imine reductase, that is used in the inventive method, which comprises a polynucleotide sequence having at least 80%, more preferably 85%, even more preferably 86%, even more preferably 87%, even more preferably 88%, even more preferably 89%, even more preferably 90%, even more preferably 91, even more preferably 92%, even more preferably, even more preferably 93%, even more preferably 94, even more preferably 95%, even more preferably 96%, even more preferably 97%, even more preferably 98% and most preferably 99% sequence identity to the nucleic acid sequence selected from SEQ ID NO: 1-299.

Further, the expression vector may comprise a nucleic acid encoding a polypeptide having the enzymatic activity of an imine reductase, that is used in the inventive method, which comprises a polynucleotide sequence having at least 80%, more preferably 85%, even more preferably 86%, even more preferably 87%, even more preferably 88%, even more preferably 89%, even more preferably 90%, even more preferably 91, even more preferably 92%, even more preferably, even more preferably 93%, even more preferably 94, even more preferably 95%, even more preferably 96%, even more preferably 97%, even more preferably 98% and most preferably 99% sequence identity to the nucleic acid sequence selected from SEQ ID Nos.: 1, 7, 22, 25, 26, 39, 47, 58, 75, 123, 135 and 160.

Further, the expression vector may comprise a nucleic acid encoding a polypeptide having the enzymatic activity of an imine reductase, that is used in the inventive method, which comprises a polynucleotide sequence having at least 80%, more preferably 85%, even more preferably 86%, even more preferably 87%, even more preferably 88%, even more preferably 89%, even more preferably 90%, even more preferably 91, even more preferably 92%, even more preferably, even more preferably 93%, even more preferably 94, even more preferably 95%, even more preferably 96%, even more preferably 97%, even more preferably 98% and most preferably 99% sequence identity to the nucleic acid sequence of SEQ ID NO: 135.

The expression vector may also comprise a nucleic acid consisting of a nucleotide sequence encoding the naturally occurring imine reductase polypeptide of SEQ ID Nos.: 300-598.

The expression vector may further comprise a nucleic acid encoding a polypeptide having an imine reductase activity capable of converting a carbonyl compound of formula (III) and amine substrate compound of formula (IV), to an amine product compound of formula (I) and/or an imine compound of formula (II) or (IIA) to amine compound of formula (IA) or (IB), wherein the polypeptide comprises an amino acid sequence having at least 80%, more preferably 85%, more preferably 86%, more preferably 87%, more preferably 88%, more preferably 89%, more preferably 90%, more preferably 91%, more preferably 92%, more preferably 93%, more preferably 94%, more preferably 95%, more preferably 96%, more preferably 97%, more preferably 98%, more preferably 99% or more sequence identity to the amino acid sequence of SEQ ID Nos.: 300-598.

The expression vector, as described herein, preferably contains one or more selectable markers, which permit easy selection of transformed cells. A selectable marker is a gene the product of which provides for biocide or viral resistance, resistance to heavy metals, prototrophy to auxotrophs, and the like. Examples of bacterial selectable markers are the dal genes from Bacillus subtilis or Bacillus licheniformis or markers, which confer antibiotic resistance such as ampicillin, kanamycin, chloramphenicol or tetracycline resistance. Suitable markers for yeast host cells are ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase) and trpC (anthranilate synthase), as well as equivalents thereof. Embodiments for use in an Aspergillus cell include the amdS and pyrG genes of Aspergillus nidulans or Aspergillus oryzae and the bar gene of Streptomyces hygroscopicus.

Thus, the expression vector, as described herein, comprises
a) a nucleic acid comprising a nucleotide sequence encoding the naturally occurring imine reductase polypeptide selected from SEQ ID NO: 300-598; and
b) a selectable marker.

Alternatively, the expression vector, as described herein, may comprise
a) a nucleic acid comprising a polynucleotide sequence having at least 80% sequence identity to the nucleic acid sequence selected from SEQ ID NO: 300-598; and
b) a selectable marker.

Further, the expression vector, as described herein, may comprise
a) a nucleic acid comprising a polynucleotide sequence having at least 80% sequence identity to the nucleic acid sequence selected from SEQ ID NO: 1, 7, 22, 25, 26, 39, 47, 58, 75, 123, 135 and 160; and
b) a selectable marker.

Also, the expression vector, as described herein, may comprise
a) a nucleic acid comprising a polynucleotide sequence having at least 80% sequence identity to the nucleic acid sequence of SEQ ID NO: 135; and
b) a selectable marker.

The expression vector may be further constructed by ligating a nucleic acid encoding a polypeptide having an imine reductase activity into the commercially available vector selected from pET-28b, pET-32b, pET-3b, pET-9b, pET-14b, pACYCDuet-1, pETDuet-1, pT7-FLAG-1 pTAC-MAT-Tag-1 and pET-16b. The pET-16b vector (SEQ ID NO: 609) includes an N-terminal His.Tag sequence, a Factor Xa site and three cloning sites.

Thus, the expression vector may comprise
a) a nucleic acid comprising a polynucleotide sequence having at least 80% sequence identity to the polynucleotide sequence selected from SEQ ID NO: 1-300; and
b) a vector selected from selected from pET-28b, pET-32b, pET-3b, pET-9b, pET-14b, pACYCDuet-1, pETDuet-1, pT7-FLAG-1 pTAC-MAT-Tag-1 and pET-16b.

Also, the expression vector, as described herein, may comprise
a) a nucleic acid comprising a polynucleotide sequence having at least 80% sequence identity to the polynucleotide sequence selected from SEQ ID NO: 1-300; and
b) a pET-16b vector having the polynucleotide sequence of SEQ ID NO: 609.

The polypeptides having an imine reductase activity and corresponding nucleic acids can be obtained using methods used by those skilled in the art. Engineered variants of the naturally occurring polypeptide having imine reductase activity described herein can be obtained by subjecting the nucleic acid encoding the naturally occurring imine reductase (SEQ ID Nos.: 300-598) to mutagenesis and/or directed evolution methods.

Where the sequence of the polypeptides having an imine reductase activity is known, the nucleic acids encoding the enzyme can be prepared by standard solid-phase methods, according to known synthetic methods, or PCR amplification. For instance, fragments of up to about 100 bases can be individually synthesized, then joined (e.g., by enzymatic or chemical litigation methods, or polymerase mediated methods) to form any desired continuous sequence. For example, nucleic acids encoding portions of the polypeptides having an imine reductase activity can be prepared by chemical synthesis using, e.g., the classical phosphoramidite method, as it is typically practiced in automated synthetic methods. According to the phosphoramidite method, oligonucleotides are synthesized, i.e. in an automatic DNA synthesizer, purified, annealed, ligated and cloned in appropriate vectors. In addition, essentially any nucleic acid can be obtained from any of a variety of commercial sources. Additional variations can be created by synthesizing oligonucleotides containing deletions, insertions, and/or substitutions, and combining the oligonucleotides in various permutations to create polypeptides having an imine reductase activity with improved properties.

The polypeptides used in the inventive method can be prepared by expressing the polypeptide in a suitable host cell, which comprises the corresponding nucleic acid encoding the polypeptide. In the host cell, the nucleic acid encoding the polypeptide used in the inventive method is operatively linked to one or more control sequences for expression of the polypeptide having imine reductase activity within the host cell.

Host cells for use in expressing the polypeptides encoded by the expression vectors described herein are well known in the art and include but are not limited to, bacterial cells, such as E. coli, Bacillus subtilis, Streptomyces and Salmonella typhimurium cells; fungal cells, such as yeast cells (e.g. Saccharomyces cerevisiae or Pichia pastoris (ATCC Accession No. 201178)); insect cells such as Drosophila S2 and Spodoptera Sf9 cells; animal cells such as CHO, COS, BHK, 293 and Bowes melanoma cells and plant cells. Exemplary host cells are Escherichia coli W3110 (ΔfhuA), BL21 and E. coli BL21 AI.

Appropriate culture mediums and growth conditions for the above-described host cells are well known in the art. Polynucleotides for expression of the imine reductase may be introduced into cells by various methods known in the art. Techniques include among others, electroporation, biolistic particle bombardment, liposome mediated transfection, calcium chloride transfection, and protoplast fusion.

Thus, the polypeptide having the enzymatic activity of an imine reductase may be prepared by a method comprising:
(a) providing a nucleic acid comprising a polynucleotide sequence having at least 80% sequence identity to a polynucleotide sequence selected from SEQ ID NO: 1-299, which encodes a polypeptide having the enzymatic activity of an imine reductase, and
(b) expressing the polypeptide encoded by the nucleic acid.

The polypeptide having the enzymatic activity of an imine reductase may also be prepared by a method comprising
(a) providing a nucleic acid comprising a polynucleotide sequence having at least 80% sequence identity to a polynucleotide sequence selected from SEQ ID NO: 1, 7, 22, 25, 26, 39, 47, 58, 75, 123, 135 and 160, which encodes a polypeptide having the enzymatic activity of an imine reductase, and
(b) expressing the polypeptide encoded by the nucleic acid.

The polypeptide having the enzymatic activity of an imine reductase may also be prepared by a method comprising
(a) providing a nucleic acid comprising a polynucleotide sequence having at least 80% sequence identity to SEQ ID NO: 135, which encodes a polypeptide having the enzymatic activity of an imine reductase, and
(b) expressing the polypeptide encoded by the nucleic acid.

The polypeptide having the enzymatic activity of an imine reductase may also be prepared by a method comprising
(a) providing a nucleic acid encoding a polypeptide comprising an amino acid sequence of at least 80% sequence identity to an amino acid sequence selected from SEQ ID NO: 300-598 and which has the enzymatic activity of an imine reductase, and
(b) expressing the polypeptide encoded by the nucleic acid.

The polypeptide having the enzymatic activity of an imine reductase may also be prepared by a method comprising
(a) providing a nucleic acid encoding a polypeptide comprising an amino acid sequence of at least 80% sequence identity to an amino acid sequence selected from SEQ ID NO: 300-598 and wherein the polypeptide is capable of converting a carbonyl compound of formula (III) and amine substrate compound of formula (IV), to an amine product compound of formula (I) and/or an imine compound of formula (II) or (IIA) to amine compound of formula (IA) or (IB), and
(b) expressing the polypeptide encoded by the nucleic acid.

Alternatively, the polypeptide having the enzymatic activity of an imine reductase may also be prepared by a method comprising
(a) providing a nucleic acid encoding a polypeptide comprising an amino acid sequence of at least 80% sequence identity to an amino acid sequence selected from SEQ ID NO: 300-598 and wherein the polypeptide is capable of converting a carbonyl compound of formula (III) and amine substrate compound of formula (IV), to an amine product compound of formula (I) and/or an imine compound of formula (II) or (IIA) to amine compound of formula (IA) or (IB), and
(b) inserting the nucleic acid of (a) into a suitable host cell and expressing said nucleic acid in order to express a polypeptide having the enzymatic activity of an imine reductase.

The polypeptide having the enzymatic activity of an imine reductase may also be prepared by a method comprising
(a) providing a nucleic acid comprising a polynucleotide sequence having at least 80% sequence identity to a polynucleotide sequence selected from SEQ ID NO: 1-299, which encodes a polypeptide having the enzymatic activity of an imine reductase, and
(b) inserting the nucleic acid of (a) into a suitable host cell and expressing said nucleic acid in order to express a polypeptide having the enzymatic activity of imine reductase.

In some embodiments, the method for preparing or manufacturing a polypeptide having the enzymatic activity of an imine reductase further comprises the step of isolating the polypeptide. The polypeptides expressed in appropriate cells can be isolated (or recovered) from the host cells, the culture medium and/or expression medium using any one or more of the well known techniques used for protein purification, including, among others, lysozyme treatment, sonication, filtration, salting-out, ultra-centrifugation, and chromatography. Suitable solutions for lysing and the high efficiency extraction of proteins from bacteria, such as E. coli, are commercially available, such as CelLytic B™ from Sigma-Aldrich. Chromatographic techniques for isolation of the imine reductase polypeptides include, among others, reverse phase chromatography high performance liquid chromatography, ion exchange chromatography, gel electrophoresis, and affinity chromatography.

Thus, the polypeptide having the enzymatic activity of an imine reductase may also be prepared by a method comprising
(a) providing a nucleic acid comprising a polynucleotide sequence having at least 80% sequence identity to a polynucleotide sequence selected from SEQ ID Nos: 1-299, which encodes a polypeptide having the enzymatic activity of an imine reductase,
(b) inserting the nucleic acid of (a) into a suitable host cell and expressing said nucleic acid in order to express a polypeptide having the enzymatic activity of an imine reductase, and
(c) isolating the expressed polypeptide.

### DESCRIPTION OF THE FIGURES

FIG. 1: illustrates the reaction sequence of the β-hydroxyaspartate pathway (BHAP) in Paracoccus denitrificans for the conversion of glyoxylate via the unstable iminosuccinate (shown in brackets) into oxaloacetate. The italic numbers represent the enzyme catalyzing the respective reaction: 1: aspartate-glyoxylate transaminase; 2: (2R,3S)-β-hydroxyaspartate aldolase (BHAA); 3: (2R,3S)-β-hydroxyaspartate dehydratase (BHAD) and 4: iminosuccinate reductase (ISRed). The net equation of the pathway is shown below the scheme.

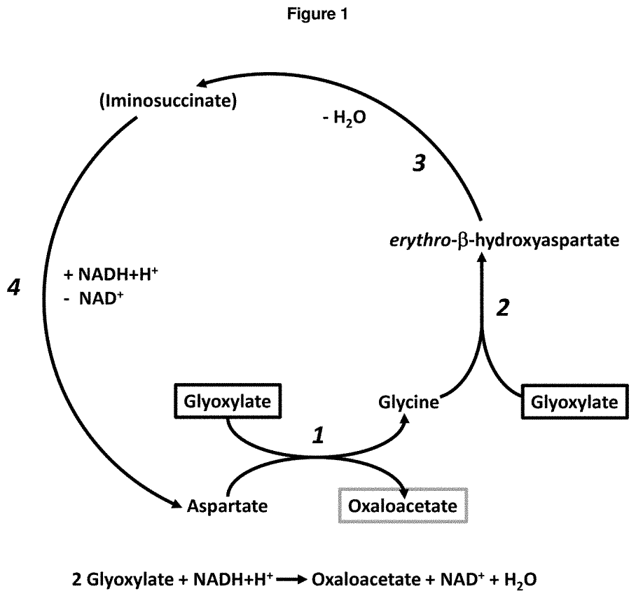

FIG. 2: shows different reactions for the preparation of amines. FIG. 2A refers to the reductive amination of a ketone or aldehyde III and a secondary amine IV in order to obtain a tertiary amine I; FIG. 2B shows the reduction of a secondary imine compound II to a secondary amine compound IA; and FIG. 2C is directed to the inventive reduction of a primary imine compound IIA to a primary amine compound IB. The asterisks in formulae IA and IB indicate that a stereocenter is formed at the carbon atom attached to the amine nitrogen atom in case a chiral or pro-chiral imine compound was used.

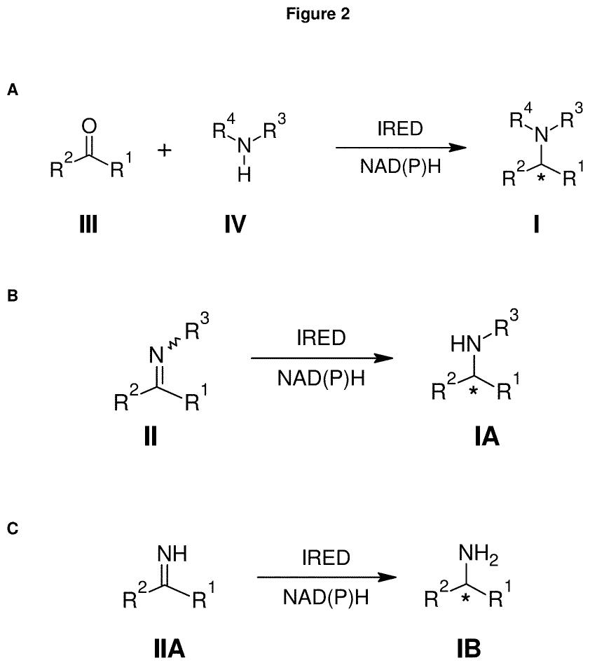

FIG. 3: LC-MS analysis of monodeuterated L-aspartate formed of glycine and glyoxylate by the BHAA and BHAD enzymes in the presence of varying amounts of NaCNBH₃ in D₂O according to Example 3.

FIG. 4: LC-MS analysis of L-aspartate and (2R,3S)-β-hydroxyaspartate formed of glycine and glyoxylate by the BHAA, BHAD and Red enzymes according to Example 3.

FIG. 5: LC-MS-based analysis of the reactions catalyzed by β-hydroxyaspartate dehydratase (BHAD) and iminosuccinate reductase (ISRed). An overview of the relevant reactions is given in (A). To demonstrate that BHAD produces iminosuccinate, (2R,3S)-β-hydroxyaspartate (light grey) is incubated in ²H₂O with BHAD and with/without NaBH₃CN to yield monodeuterated aspartate (medium grey; B) or oxaloacetate (white; C). To demonstrate that ISRed reduces iminosuccinate to aspartate, (2R,3S)-β-hydroxyaspartate is incubated with BHAD and with/without iminosuccinate reductase to yield aspartate (dark grey; D) or oxaloacetate (white; E). n=3, error bars depict standard deviation.

FIG. 6: Michaelis-Menten kinetics for the Red enzyme, with iminosuccinate synthesized in situ from BHA and varying amounts of the BHAD enzyme. The curve was fitted in GraphPad Prism 7. The vₘₐₓ is 270±10 U mg⁻¹ (n=1).

FIG. 7: shows the crystal structure of the iminosuccinate reductase (ISRed). a, Cartoon representation of the iminosuccinate reductase homodimer (PDB ID 6QKH) with superimposed protein surface (side view—left panel, top view—right panel). b, Superimposition of monomers of iminosuccinate reductase and L-alanine dehydrogenase (PDB ID 1OMO) with bound NAD⁺. RMS of 1.287 Å over 241 Cα-atoms. c, Superimposition of the active sites of iminosuccinate reductase and L-alanine dehydrogenase. Although the binding of the nicotine amide moiety appears to be similar in both cases, the residues lining the active site from above differ substantially. These residues are located in the β-strands which also form the dimerization interface (see a for comparison) and are likely to be involved in the binding and orientation of the respective substrates above the nicotine amide moiety.

The following examples are included to demonstrate preferred embodiments of the invention. It should be appreciated by those skilled in the art that the techniques disclosed in the examples, which follow represent techniques discovered by the inventor to function well in the practice of the invention, and thus can be considered to constitute preferred modes for its practice. However, those skilled in the art should, in light of the present disclosure, appreciate that many changes can be made in the specific embodiments, which are disclosed and still obtain a like or similar result without departing from the spirit and scope of the invention.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

### EXAMPLES

### Abbreviations and Acronyms

IRed imine reductase
ISRed iminosuccinate reductase
BHA (2R,3S)-β-hydroxyaspartate
BHAA (2R,3S)-β-hydroxyaspartate aldolase
BHAD (2R,3S)-β-hydroxyaspartate dehydratase
DNA desoxyribo nucleic acid

### Chemicals & Reagents

Unless otherwise stated, all chemicals and reagents were acquired from Sigma-Aldrich, and were of the highest purity available.

### Example 1: Construction of Expression Vectors for Heterologous Expression of the Imine Reductase, β-Hydroxyaspartate Aldolase and β-Hydroxyaspartate Dehydratase Polypeptide

The gene encoding for the imine reductase enzyme from Paracoccus denitrificans DSM 413 (IRed; nucleic acid sequence shown in SEQ ID NO: 135; amino acid sequence shown in SEQ ID NO: 434) was cloned into the standard expression vector pET16b (commercially available from Merck Millipore). To this end, the Red gene was amplified from genomic DNA of Paracoccus denitrificans DSM 413 with the primers

(SEQ ID NO: 603)
5′-GACGCCTCATATGCTCGTCGTCGCCGAAAAG-3′
(SEQ ID NO: 604)
5′-GCCACTCCTCGAGTCAGATCTCGACCTCTTG-3′

The resulting PCR product was digested with the endonucleases NdeI and XhoI and ligated into the expression vector pET16b to create a vector for heterologous expression of Red.

The gene encoding for the β-hydroxyaspartate aldolase enzyme from Paracoccus denitrificans DSM 413 (BHAA; nucleic acid sequence shown in SEQ ID NO: 599; amino acid sequence shown in SEQ ID NO: 600) was cloned into the standard expression vector pET16b (commercially available from Merck Millipore). To this end, the BHAA gene was amplified from genomic DNA of Paracoccus denitrificans DSM 413 with the primers

(SEQ ID NO: 605)
5′-GACGCCGCATATGAACGCGAAAACGGATTTC-3′
(SEQ ID NO: 606)
5′-GACACCTGGATCCTCAGTAGCCCTTTCCG-3′

The resulting PCR product was digested with the endonucleases NdeI and BamHI and ligated into the expression vector pET16b to create a vector for heterologous expression of BHAA.

The gene encoding for the β-hydroxyaspartate dehydratase enzyme from Paracoccus denitrificans DSM 413 (BHAD; nucleic acid sequence shown in SEQ ID NO: 601; amino acid sequence shown in SEQ ID NO: 602) was cloned into the standard expression vector pET16b (commercially available from Merck Millipore). To this end, the BHAD gene was amplified from genomic DNA of Paracoccus denitrificans DSM 413 with the primers

(SEQ ID NO: 607)
5′-GACGCTGCATATGTATATCCCGACCTATGAG-3′
(SEQ ID NO: 608)
5′-GACACTCGGATCCTCAGTTCCACGGCAGCTTG-3′

The resulting PCR product was digested with the endonucleases NdeI and BamHI and ligated into the expression vector pET16b to create a vector for heterologous expression of BHAD.

### Example 2: Heterologous Expression and Purification of Recombinant Proteins

For the heterologous overexpression of the Red, BHAA and BHAD enzymes, respectively, the corresponding plasmid encoding the respective enzyme was first transformed into chemically competent E. coli BL21 AI cells. The cells transformed with the respective plasmid encoding one of said enzymes were then grown on LB agar plates containing 100 μg mL⁻¹ ampicillin at 37° C. overnight. A starter culture in selective LB medium was then inoculated from a single colony on the next day and left to grow overnight at 37° C. in a shaking incubator. The starter culture was then used on the next day to inoculate an expression culture in selective TB medium in a 1:100 dilution. The expression culture was grown at 37° C. in a shaking incubator to an OD₆₀₀ of 0.5 to 0.7, induced with 0.5 mM IPTG and 0.2% L-arabinose and grown overnight at 18° C. in a shaking incubator.

Cells were harvested at 6,000×g for 15 min and cell pellets were stored at −20° C. until purification of enzymes. Cell pellets were resuspended in two-fold volume of buffer A (300 mM NaCl, 25 mM Tris pH 8.0, 15 mM imidazole, 1 mM β-mercaptoethanol, 0.1 mM MgCl₂, 0.01 mM pyridoxalphosphate (PLP), and one tablet of SIGMAFAST™ protease inhibitor cocktail, EDTA-free (Sigma-Aldrich) per L). The cell suspension was treated with a sonicator in order to lyse the cells and centrifuged at 50,000×g and 4° C. for 1 h. The supernatant was loaded onto 1 ml Protino® Ni-NTA Agarose (Macherey-Nagel) in a gravity column, which had previously been equilibrated with 5 column volumes of buffer A. The column was washed with 20 column volumes of buffer A and 5 column volumes of 85% buffer A and 15% buffer B and the respective protein was eluted with buffer B (buffer A, but with 500 mM imidazole). The eluate was desalted using PD-10 desalting columns (GE Healthcare) and buffer C (100 mM NaCl, 25 mM Tris pH 8.0, 1 mM MgCl₂, 0.01 mM PLP, 0.1 mM DTT). The respective purified enzymes were stored at −20° C. in buffer C containing 50% glycerol.

### Example 3: Enzyme Assays

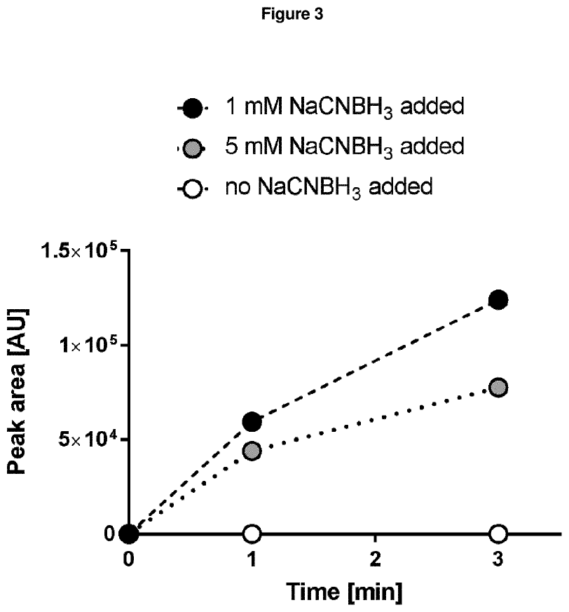

The enzyme assay to generate iminosuccinate from glyoxylate and glycine (catalyzed by the BHAA and BHAD enzymes) and further chemical reduction of iminosuccinate to aspartate with the reducing agent NaCNBH₃ was performed at 30° C. in a total volume of 600 μl. The reaction mixture contained 50 mM Tris pH 7.5, 1 mM glycine, 1 mM glyoxylate, 0.1 mM PLP, 1 mM MgCl₂, 60 μg BHAA enzyme, 5.4 μg BHAD enzyme and varying amounts of NaCNBH₃ (0, 1 or 5 mM, respectively). The reaction was carried out in deuterated water (D₂O). 180 μL aliquots were taken at time points 0, 1 and 3 minute(s) and the reaction was immediately stopped by addition of formic acid (4% final concentration). The samples were centrifuged at 17,000×g and 4° C. for 15 min and the supernatant diluted 1:4 in double-distilled water for LC-MS analysis (see FIG. 3).

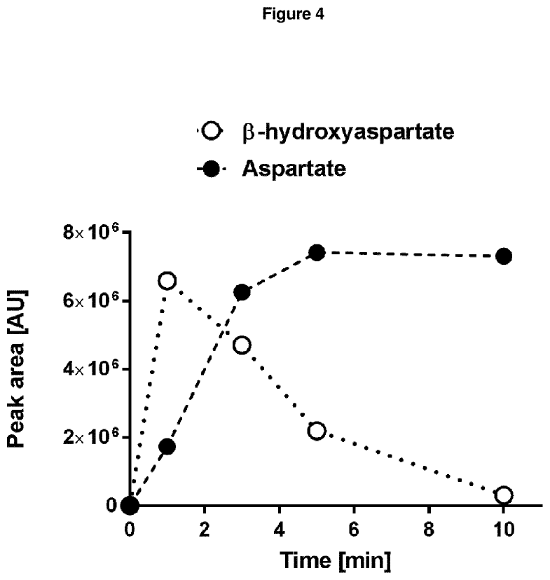

The enzyme assay to generate aspartate from glyoxylate and glycine (catalyzed by the BHAA, BHAD and ISRed enzymes) was performed at 30° C. in a total volume of 1 ml. The reaction mixture contained 50 mM MOPS/KOH pH 7.5, 1 mM glycine, 1 mM glyoxylate, 0.4 mM NADH, 0.1 mM PLP, 1 mM MgCl₂, 100 μg BHAA enzyme, 9 ug BHAD enzyme and 1 μg ISRed enzyme. 180 μL aliquots were taken at time points 0, 1, 3, 5 and 10 minutes and the reaction was immediately stopped by formic acid (4% final concentration). The samples were centrifuged at 17,000×g and 4° C. for 15 min and the supernatant diluted 1:4 in double-distilled water for LC-MS analysis (see FIG. 4).

Thus, when incubating glyoxylate, glycine and the required cofactors together with the BHAA, BHAD and ISRed enzymes, increasing amounts of aspartate are formed over the course of 10 min.

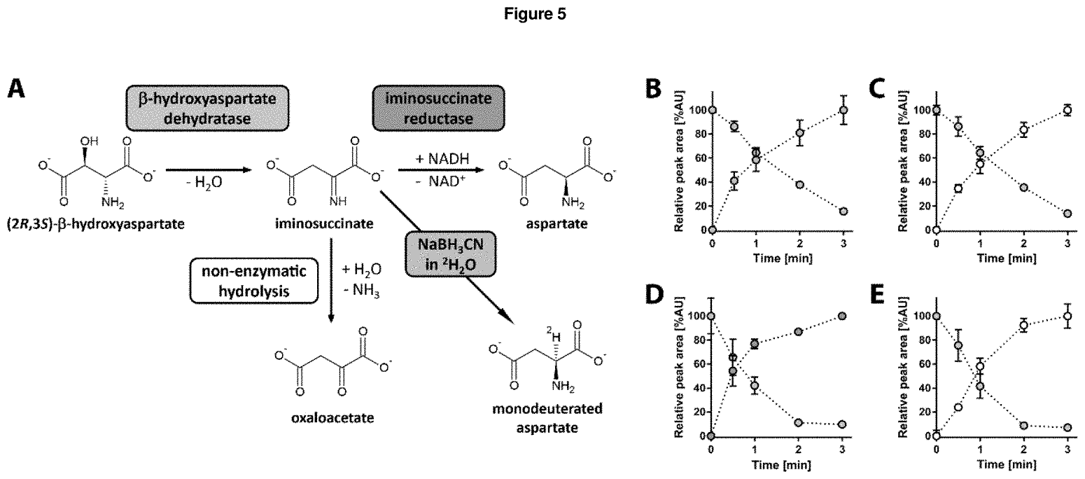

The LC-MS measurements were done using an Agilent 6550 iFunnel Q-TOF LC-MS system equipped with an electrospray ionization source set to negative ionization mode. Liquid chromatography (LC) was carried out as follows: The analytes were separated on an aminopropyl column (30 mm×2 mm, particle size 3 μm, 100 Å, Luna NH₂, Phenomenex) using a mobile phase system comprised of 95:5 20 mM ammonium acetate pH 9.3 (adjusted with ammonium hydroxide to a final concentration of approximately 10 mM)/acetonitrile (A) and acetonitrile (B). Chromatographic separation was carried out using the following gradient condition at a flow rate of 250 μl min⁻¹: 0 min 85% B; 3.5 min 0% B, 7 min, 0% B, 7.5 min 85% B, 8 min 85% B. Column oven and autosampler temperature were maintained at 15° C. The ESI source was set to the following parameters: Capillary voltage was set at 3.5 kV and nitrogen gas was used as nebulizing (20 psig), drying (13 l/min, 225 C) and sheath gas (12 l/min, 400° C.). The QTOF mass detector was calibrated prior to measurement using an ESI-L Low Concentration Tuning Mix (Agilent) with residuals and corrected residuals less than 2 ppm and 1 ppm respectively. MS data were acquired with a scan range of 50-600 m/z. Autorecalibration was carried out using 113 m/z as reference mass. Subsequent peak integration of all analytes was performed using the eMZed software (Kiefer et al. Bioinformatics, 2013, 29(7), 963-964) (see FIG. 5).

The enzyme assay to generate aspartate from β-hydroxyaspartate (catalyzed by the BHAD and ISRed enzymes) was performed at 30° C. in a total volume of 300 μl. The reaction mixture contained 100 mM potassium phosphate pH 7.5, 1 mM BHA, 0.2 mM NADH, 0.57, 2.85, 5.7 or 11.4 μg BHAD enzyme and 0.57 μg ISRed enzyme. The oxidation of NADH was followed at 340 nm on a Cary 60 UV-Vis photospectrometer (Agilent) in quartz cuvettes with a pathlength of 10 mm (Hellma Analytics). BHA (=(2R,3S)-β-hydroxyaspartate) was custom-synthesized by the company NewChem (Newcastle upon Tyne, United Kingdom), and was determined to be >95% pure by NMR analysis. Therefore, the ISRed enzyme catalyzes the conversion of iminosuccinate into L-aspartate.

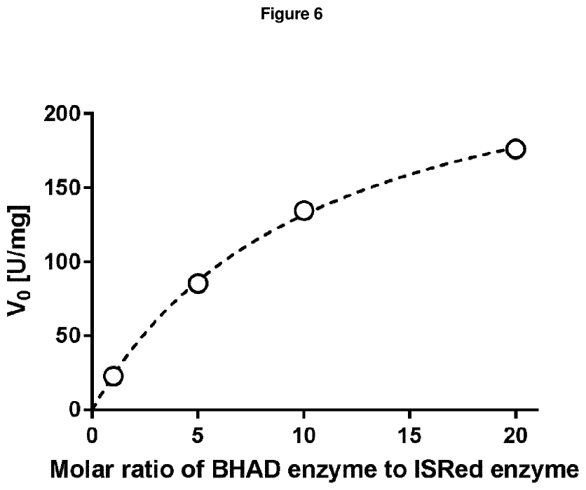

The enzyme assay to determine the initial reaction velocity of the ISRed enzymes was performed by incubating BHA and the required cofactor NADH together with a fixed amount of the ISRed enzyme and varying amounts of the BHAD enzyme. The initial velocity depends on the amount of the BHAD enzyme present in the reaction mixture, and therefore on the amount of iminosuccinate that is produced by the BHAD enzyme and is available to the ISRed enzyme, as illustrated in FIG. 6. The initial reaction velocity of the ISRed enzyme approaches a maximum in a Michaelis-Menten kinetic fit. The preliminary vₘₐₓ of the ISRed enzyme determined by this method is 270±10 U mg⁻¹.

### Example 4: Crystallization and Structure Determination of ISRed

The sitting-drop vapor-diffusion method was used for crystallization at 16° C. Purified ISRed (5 mg ml⁻¹) was mixed in a 1:1 ratio with solution B containing 20% PEG 3350, 0.06 M BIS-TRIS propane, and 0.04 M citric acid, pH 6.4 (final drop volume 4 μL). Reservoirs were filled with 114 μL of solution B. Crystals appeared within 12 days. Crystals were briefly soaked in mother liquor supplemented with 12 mM NAD⁺ and 40% MPD (2-Methyl-2,4-pentanediol) for cryoprotection before freezing in liquid nitrogen.

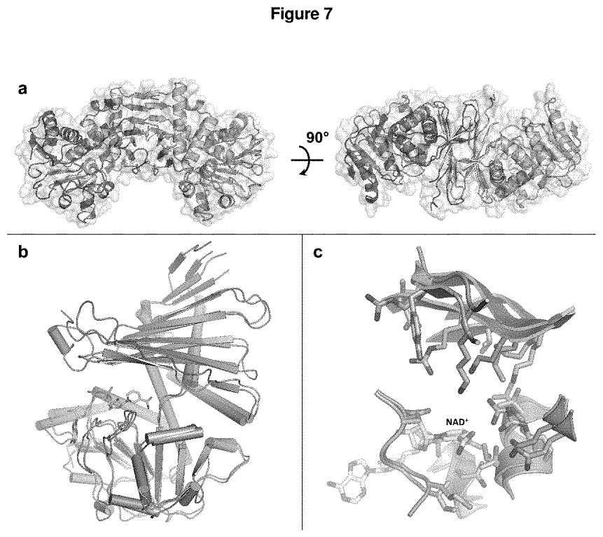

X-ray diffraction data were collected at the beamlines ID29 and ID30B of the ESRF (Grenoble, France). The data was processed with the XDS (Kabsch, W. (2010). “Xds.” Acta Crystallogr D Biol Crystallogr 66(Pt 2): 125-132). (BUILT 20180126) and CCP4 7.0 software packages (Winn et al. “Overview of the CCP4 suite and current developments.” Acta Crystallogr D Biol Crystallogr 67(Pt 4): 235-242). The structures were solved by molecular replacement. A homology model was made based on the structure of L-alanine dehydrogenase (PDB ID 1OMO) (Gallagher et al. “Structure of alanine dehydrogenase from Archaeoglobus: active site analysis and relation to bacterial cyclodeaminases and mammalian mu crystallin.” J Mol Biol 342(1): 119-130.) using Swiss-Model (Waterhouse et al. “SWISS-MODEL: homology modelling of protein structures and complexes.” Nucleic Acids Res 46(W1): W296-W303.). This homology model was then used as search model for the molecular replacement. The molecular replacement was carried out using Phaser of the Phenix software package (Adams et al. PHENIX: a comprehensive Python-based system for macromolecular structure solution.” Acta Crystallogr D Biol Crystallogr 66(Pt 2): 213-221.) (version 1.14), built with Phenix.Autobuild, and refined with Phenix.Refine. Additional modeling, manual refining and ligand fitting was done in Coot (Emsley et al. “Coot: model-building tools for molecular graphics.” Acta Crystallogr D Biol Crystallogr 60(Pt 12 Pt 1): 2126-2132.) (version 0.8.9). Final positional and B-factor refinements, as well as water-picking for the BHAA structure were performed using Phenix.Refine. The structure models for BHAA and ISRed were deposited at the Protein Bata Bank in Europe (PDBe) under the PDB ID 6QKB and 6QKH, respectively. FIG. 7 was made using Pymol 1.8.

A sequence listing is attached to this application comprising the sequences of the following table:

| SEQ ID | description |
|---|---|
| 1-299 | nucleic acids encoding polypeptides having the |
| | enzymatic activity of an imine reductase |
| 300-598 | polypeptides having the enzymatic activity of |
| | an imine reductase |
| 599 | beta-hydroxyaspartate aldolase |
| 601 | beta-hydroxyaspartate dehydratase |
| 603 | PCR Primer for imine reductase polypeptide |
| | encoding gene |
| 604 | PCR Primer for imine reductase polypeptide |
| | encoding gene |
| 605 | PCR primer for beta-hydroxyaspartate |
| | aldolase polypeptide encoding gene |
| 606 | PCR primer for beta-hydroxyaspartate |
| | aldolase polypeptide encoding gene |
| 607 | PCR primer for beta-hydroxyaspartate |
| | dehydratase polypeptide encoding gene |
| 608 | PCR primer for beta-hydroxyaspartate |
| | dehydratase polypeptide encoding gene |
| 609 | pET-16b expression vector |
| 610, 611 | Conserved amino acid sequence of the iminosuccinate |
| | reductases of SEQ ID NO: 300-598 |
| 612 | Conserved amino acid sequence of the iminosuccinate |
| | reductases of SEQ ID NO: 300, 306, 321, 324, |
| | 325, 338, 346, 357, 374, 422, 434 and 459 |
| 613-616 | Conserved amino acid sequence of the iminosuccinate |
| | reductases |
| 617 | Conserved amino acid sequence of the iminosuccinate |
| | reductases of SEQ ID NO 303, 309, 310, 318, 344, 345, |
| | 347, 362, 385, 386, 396, 397, 414, 432, 434, 446, 459, |
| | 470, 472, 491, 503, 509, 513, 524, 539, 564, and 576 |
| 618 | Conserved amino acid sequence of the iminosuccinate |
| | reductases of SEQ ID NO 303, 309, 344, 345, 347, 385, |
| | 386, 396, 414, 432, 434, 446, 459, 470, 472, 503, 513, |
| | 539, 564, and 576. |
| 619 | Conserved amino acid sequence of the iminosuccinate |
| | reductases of SEQ ID NO 303, 344, 345, 347, 385, 386, |
| | 396, 414, 432, 434, 470, 472, 503, 539, and 576. |
| 620 | Conserved amino acid sequence of the iminosuccinate |
| | reductases of SEQ ID NO: 303, 344, 345, 385, 386, 396, |
| | 414, 432, 434, and 470. |
| 621 | Conserved amino acid sequence of the iminosuccinate |
| | reductases of SEQ ID NO 303, 345, 396, 432, and 434. |

What is claimed is:

1. A method of preparing a primary amine compound of general formula (IB) 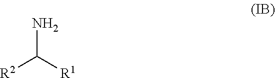
wherein R¹ and R² are independently selected from a hydrogen atom, and optionally substituted alkyl, alkenyl, alkynyl, alkoxy, carboxy, aminocarbonyl, thiocarbonyl, heteroalkyl, heteroalkenyl, heteroalkynyl, carboxyalkyl, aminoalkyl, haloalkyl, alkylthioalkyl, cycloalkyl, aryl, arylalkyl, heterocycloalkyl, heteroaryl, and heteroarylalkyl; and optionally R¹ and R² are linked to form a 3-membered to 10-membered ring;
comprising the step of:
A1) Providing an imine compound of general formula (IIA) 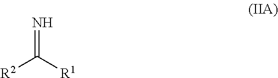
wherein R¹ and R² have the meanings as defined above; and
B1) Reacting imine compound of general formula (IIA) with a polypeptide having the enzymatic activity of an imine reductase in the presence of a cofactor, to afford primary amine of general formula (IB),
wherein the polypeptide comprises a conserved amino acid as set forth in SEQ ID NO: 610.

2. The method of claim 1, wherein
R¹ represents —CH₃, —COOR⁵, —COSR⁵, —CSSR⁵, —CONHR⁵, —CONR⁵R⁶, —CH═CH—COOR⁵, —CH═CH—COSR⁵, —CH═CH—CSSR⁵, —CH═CH—CONHR⁵, —CH═CH—CONR⁵R⁶, —C≡C—COOR⁵, —C≡C—COSR⁵, —C≡C—CSSR⁵, —C≡C—CONHR⁵ or —C≡C—CONR⁵R⁶;
R² represents —H, —X¹, —CH₃, —CF₃, -Ph, —CH₂Y¹, —CH(Y¹)Y², cyclo-C₃H₅, cyclo-C₄H₇, cyclo-C₅H₉, cyclo-C₆H₁₁, cyclo-C₇H₁₃, cyclo-C₈H₁₅, —CH(OH)X¹, —C(O)Y¹, —CH(X¹)X², —CH₂CH₂X¹, —(CH₂)₃X¹, —CH₂C(O)X¹ or —C(O)CH₂X¹
X¹ and X² are independently selected from —CN, —COOR⁷, —COOR⁸, —COSR⁷, —COSR⁸, —CSSR⁷, —CSSR⁸, —CONHR⁷, —CONHR⁸, —CONR⁷R⁹ or —CONR⁸R¹⁰,
Y¹, Y² and Y³ are independently selected from —X¹, —X², —R¹³, —R¹⁴, or —R¹⁵,
R⁵-R¹⁵ represent independently of each other —H, cyclo-C₃H₅, cyclo-C₄H₇, cyclo-C₅H₉, cyclo-C₆H₁₁, cyclo-C₇H₁₃, cyclo-C₈H₁₅, -Ph, —CH₂-Ph, —CPh₃, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)—C(CH₃)₃, —CH═CH₂, —CH₂—CH═CH₂, —C(CH₃)═CH₂, —CH═CH—CH₃, —C₂H₄—CH═CH₂, —C₇H₁₅, —C₈H₁₇, —CH₂—CH═CH—CH₃, —CH═CH—C₂H₅, —CH₂—C(CH₃)═CH₂, —CH(CH₃)—CH═CH, —CH═C(CH₃)₂, —C(CH₃)═CH—CH₃, —CH═CH—CH═CH₂, —C₃H₆—CH═CH₂, —C₂H₄—CH═CH—CH₃, —CH₂—CH═CH—C₂H₅, —CH═CH—C₃H₇, —CH₂—CH═CH—CH═CH₂, —CH═CH—CH═CH—CH₃, —CH₂NH₂, —CH₂OH, —CH₂SH, —CH₂—CH₂NH₂, —CH₂—CH₂SH, —C₆H₄—OCH₃, —C₆H₄—OH, —CH₂—CH₂—OCH₃, —CH₂—CH₂OH, —CH₂—OCH₃, —CH₂—C₆H₄—OCH₃, —CH₂—C₆H₄—OH.

3. The method of claim 1, wherein
R¹ represents —COOH; and
R² is selected from —CH₃, —COOH, —CH₂CH₃, —(CH₂)₂CH₃, —(CH₂)₃CH₃, —CH₂COOH, —CH₂CONH₂, —CH₂CH₂OH, —CH₂CH₂SH, —CH(CH₃)COOH, —(CH₂)₂COOH or —(CH₂)₂CONH₂.

4. The method of claim 1, wherein the cofactor is NADH.

5. The method of claim 1, wherein the (R)-isomer of the primary amine of general formula (IB) is obtained.

6. The method of claim 5, wherein
R¹ represents —COOH,
R² represents —CHZ¹Z², and
wherein Z¹ represents —H or —CH₃ and
Z² represents —H, —CH₃, —CH₂CH₃, —(CH₂)₂CH₃, —COOH, —CONH₂, —CH₂OH, —CH₂SH, —CH₂COOH or —CH₂CONH₂.

7. The method of claim 1, wherein the imine compound of general formula (IIA) is provided by reacting a ketone or an aldehyde compound with ammonia or an ammonium salt.

8. The method of claim 7 comprising the steps of:
A2) Providing a carbonyl compound of general formula (III) 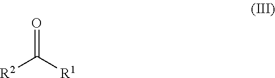
and ammonia or an ammonium salt in order to form the primary imine of general formula (IIA);
B2) Reacting the formed primary imine of general formula (IIA) with a polypeptide having the enzymatic activity of an imine reductase and a cofactor to afford the primary amine of general formula (IB),
wherein the polypeptide comprises a conserved amino acid sequence as set forth in SEQ ID NO: 610.

9. The method of claim 6, wherein the imine compound of general formula (IIA) is provided by reacting an amino alcohol compound of general formula (V) with a β-hydroxyaspartate dehydratase 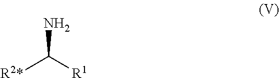
wherein R¹ represents —COOH, R²* represents —C(OH)Z¹Z² and Z¹ and Z² have the meanings as defined in claim 6.

10. The method of claim 9, wherein the β-hydroxyaspartate dehydratase comprises an amino acid sequence of at least 80% sequence identity to SEQ ID NO: 602.

11. The method of claim 1, wherein the polypeptide having the enzymatic activity of an imine reductase comprises an amino acid of at least 80% sequence identity to an amino acid sequence selected from SEQ ID NO: 300-598.

12. The method of claim 1, wherein the polypeptide having the enzymatic activity of an imine reductase comprises an amino acid sequence selected from SEQ ID NO: 300-598.

13. The method of claim 1, wherein the polypeptide having the enzymatic activity of an imine reductase comprises an amino acid sequence selected from SEQ ID NO: 300, 306, 321, 324, 325, 338, 346, 357, 374, 422, 434 and 459.

14. The method of claim 1, wherein the polypeptide having the enzymatic activity of an imine reductase comprises an amino acid sequence as set forth in SEQ ID NO: 434.

15. The method of claim 1, wherein R¹ represents —COOH and R² represents —CH₂COOH.
